SEQUENCE LISTING (43 sequences)

SEQ ID NO: 1 - protein, 11 aa, Artificial Sequence
  Synthetic Construct
RASQSIRSYLN

SEQ ID NO: 2 - protein, 7 aa, Artificial Sequence
  Synthetic Construct
AASSLQS

SEQ ID NO: 3 - protein, 10 aa, Artificial Sequence
  Synthetic Construct
QQTYSNPPIT

SEQ ID NO: 4 - protein, 7 aa, Artificial Sequence
  Synthetic Construct
GFTFDDY

SEQ ID NO: 5 - protein, 6 aa, Artificial Sequence
  Synthetic Construct
SWNSGS

SEQ ID NO: 6 - protein, 15 aa, Artificial Sequence
  Synthetic Construct
DMSGYGHYGKYGMDV

SEQ ID NO: 7 - protein, 215 aa, Artificial Sequence
  Synthetic Construct
DIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 227 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 8 aa, Artificial Sequence
  Synthetic Construct
AASGGAGS

SEQ ID NO: 10 - protein, 12 aa, Artificial Sequence
  Synthetic Construct
GAVPLSLYGAGG

SEQ ID NO: 11 - protein, 12 aa, Artificial Sequence
  Synthetic Construct
SSGASASAAGGS

SEQ ID NO: 12 - protein, 7 aa, Artificial Sequence
  Synthetic Construct
GRTFRNY

SEQ ID NO: 13 - protein, 6 aa, Artificial Sequence
  Synthetic Construct
TWVGAS

SEQ ID NO: 14 - protein, 15 aa, Artificial Sequence
  Synthetic Construct
GRGIVAGRIPAEYAD

SEQ ID NO: 15 - protein, 122 aa, Artificial Sequence
  Synthetic Construct
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTV

SEQ ID NO: 16 - protein, 586 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSGAVPLSLYGAGGDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 590 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGLVQAGHSLRLSCVGSGSRFDNYAMGWFRQAPGKEREFVAAISWSSGTTRYLDTVKGRFTISRDNAKSTVYLQMNSLKPEDTAVYYCAARYQPRYYDSGDMDGYEYDNWGQGTQVTVSSGAVPLSLYGAGGDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 583 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGSVQAGGSLTLSCTASGLLFRLASMGWYRQAPGKERELIATITVGGKTNYKDSVQGRFIITRDNTGDNTKSTVTLQMNRLKPEDTAVYYCNTASPAVGADTWGQGTRVTVSSGAVPLSLYGAGGDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 586 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSSSSGASASAAGGSDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 590 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGLVQAGHSLRLSCVGSGSRFDNYAMGWFRQAPGKEREFVAAISWSSGTTRYLDTVKGRFTISRDNAKSTVYLQMNSLKPEDTAVYYCAARYQPRYYDSGDMDGYEYDNWGQGTQVTVSSSSGASASAAGGSDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 583 aa, Artificial Sequence
  Synthetic Construct
EVQLVESGGGLVQPGRSLRLSCAASGFTFDDYTMHWVRQAPGKGLEWVSDISWNSGSIGYADSVKGRFTVSRDNAKNSLYLQMNSLRGEDTAIYYCAKDMSGYGHYGKYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAASGGAGSDVQLQESGGGSVQAGGSLTLSCTASGLLFRLASMGWYRQAPGKERELIATITVGGKTNYKDSVQGRFIITRDNTGDNTKSTVTLQMNRLKPEDTAVYYCNTASPAVGADTWGQGTRVTVSSSSGASASAAGGSDIQMTQSPILLSASVGDRVTITCRASQSIRSYLNWYQQRTNGSPRLLIYAASSLQSGVPSRFSGSGSGTDFTLTINSLQPDDEADYYCQQTYSNPPITFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 6 aa, Artificial Sequence
  Synthetic Construct
SRFDNY

SEQ ID NO: 23 - protein, 5 aa, Artificial Sequence
  Synthetic Construct
WSSGT

SEQ ID NO: 24 - protein, 18 aa, Artificial Sequence
  Synthetic Construct
YQPRYYDSGDMDGYEYDN

SEQ ID NO: 25 - protein, 126 aa, Artificial Sequence
  Synthetic Construct
DVQLQESGGGLVQAGHSLRLSCVGSGSRFDNYAMGWFRQAPGKEREFVAAISWSSGTTRYLDTVKGRFTISRDNAKSTVYLQMNSLKPEDTAVYYCAARYQPRYYDSGDMDGYEYDNWGQGTQVTV

SEQ ID NO: 26 - protein, 7 aa, Artificial Sequence
  Synthetic Construct
LLFRLAS

SEQ ID NO: 27 - protein, 6 aa, Artificial Sequence
  Synthetic Construct
NTGDNT

SEQ ID NO: 28 - protein, 8 aa, Artificial Sequence
  Synthetic Construct
SPAVGADT

SEQ ID NO: 29 - protein, 119 aa, Artificial Sequence
  Synthetic Construct
DVQLQESGGGSVQAGGSLTLSCTASGLLFRLASMGWYRQAPGKERELIATITVGGKTNYKDSVQGRFIITRDNTGDNTKSTVTLQMNRLKPEDTAVYYCNTASPAVGADTWGQGTRVTV

SEQ ID NO: 30 - protein, 14 aa, Artificial Sequence
  Synthetic Construct
SSSSGASASAAGGS

SEQ ID NO: 31 - protein, 14 aa, Artificial Sequence
  Synthetic Construct
SSGAVPLSLYGAGG

SEQ ID NO: 32 - protein, 17 aa, Artificial Sequence
  Synthetic Construct
KSSQSLLNSRTRKNYLA

SEQ ID NO: 33 - protein, 7 aa, Artificial Sequence
  Synthetic Construct
WASTRES

SEQ ID NO: 34 - protein, 8 aa, Artificial Sequence
  Synthetic Construct
KQSYILRT

SEQ ID NO: 35 - protein, 7 aa, Artificial Sequence
  Synthetic Construct
GYTFTNY

SEQ ID NO: 36 - protein, 6 aa, Artificial Sequence
  Synthetic Construct
YCGDGS

SEQ ID NO: 37 - protein, 10 aa, Artificial Sequence
  Synthetic Construct
DWAGSYFFDF

SEQ ID NO: 38 - protein, 112 aa, Artificial Sequence
  Synthetic Construct
DIQMTQSPSSLSASVGDRVTITCKSSQSLLNSRTRKNYLAWYQQKPGKAPKLLIYWASTRESGVPSRFSGSGSGTDFTFTISSLQPEDIATYYCKQSYILRTFGGGTKVEIK

SEQ ID NO: 39 - protein, 119 aa, Artificial Sequence
  Synthetic Construct
QVQLQESGPGLVKPSETLSLTCTASGYTFTNYYIHWVRQSPGKGLEWIGWIYCGDGSTKFNEQFKDRTTLSADHSKTQASLKLSSVTAADTAIYFCARDWAGSYFFDFWGQGTMVTVSS

SEQ ID NO: 40 - protein, 112 aa, Artificial Sequence
  Synthetic Construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYILRTFGGGTKLEIK

SEQ ID NO: 41 - protein, 119 aa, Artificial Sequence
  Synthetic Construct
QVQLQQSGPELVKPGASVRMSCKASGYTFTNYYIHWVRQRPGQGLEWIGWIYCGDGSTKFNEQFKDRTTLTADHSSSTAYMLLSSLTSEDSAIYFCARDWAGSYFFDFWGQGTTLTVSS

SEQ ID NO: 42 - protein, 112 aa, Artificial Sequence
  Synthetic Construct
DIVMSQSPSSLAVSAGEKVTISCKSSQSLLNNRTRKNYLAWYQQKPGQSPKLLIYWASTRESGVPDRFTGSGSGTDFTLTISSVQAEDLAVYYCKQSYILRTFGGGTKLEIK

SEQ ID NO: 43 - protein, 117 aa, Artificial Sequence
  Synthetic Construct
QVQLQQSGPDLVRPGASVKMSCKASGYTFTNYYVHWLNQRPGQGLEWIGWIYPGDGSTKFNEKFKGKTTLTADKSSSTAYILLSSLTSADSAIYFCTRDAGYYFDVWGAGTTVTVSS

(19) United States
(12) Patent Application Publication
(10) Pub. No.: US 2026/0008864 A1
(43) Pub. Date: Jan. 8, 2026

(54) BISPECIFIC ANTI-CD38-CD3 BINDERS

(71) Applicant: CITY OF HOPE, Duarte, CA (US)

(72) Inventors: Guido MARCUCCI, Duarte, CA (US); Mariam MURTADHA, Duarte, CA (US); John C. WILLIAMS, Monrovia, CA (US); Miso PARK, Duarte, CA (US); Flavia PICHIORRI, Duarte, CA (US)

(21) Appl. No.: 18/250,918

(22) PCT Filed: Oct. 28, 2021
(86) PCT No.: PCT/US2021/057143

§ 371 Date: Dec. 12, 2023

Related U.S. Application Data
(60) Provisional application No. 63/106,840, filed on Oct. 28, 2020.
(60) Provisional application No. 63/261,944, filed on Sep. 30, 2021.

(51) Int. Cl. C07K 16/28; A61K 39/00; A61P 35/02

(52) U.S. Cl. CPC C07K 16/2896; A61P 35/02; C07K 16/2809; A61K 2039/505; C07K 2317/31; C07K 2317/52; C07K 2317/55; C07K 2317/569; C07K 2317/732; C07K 2317/92; C07K 2319/50

(57) ABSTRACT

Provided herein are, inter alia, novel peptide compositions having bi-specific binding capabilities useful for therapeutic and diagnostic purposes. The peptide compositions provided herein are polypeptide conjugates including at an anti-CD3 binding domain and a CD38 binding domain and are therefore able to target (bind) CD3 and CD38 at the same time. The peptide compositions provided herein are highly efficient binders and can be produced at very high yields and are therefore easy to manufacture.

### CROSS-REFERENCES TO RELATED APPLICATIONS

This application is a U.S. National Phase application under 35 U.S.C. § 371 of International Patent Application Number PCT/US2021/057143, filed Oct. 28, 2021, which claims the benefit of U.S. Provisional Application No. 63/106,840, filed Oct. 28, 2020, and U.S. Provisional Application No. 63/261,944, filed Sep. 30, 2021, which are incorporated herein by reference in entirety and for all purposes.

### REFERENCE TO AN ELECTRONIC SEQUENCE LISTING

The contents of the electronic sequence listing (048440-786N01US_SequenceListing.txt; Size: 50,189 bytes; and Date of Creation: May 9, 2023) is hereby incorporated by reference in its entirety.

### BACKGROUND

Bispecific T cell engagers are bivalent molecules that bridge tumor cells and T cells. Initial strategies to produce said bispecific molecules included expressing all four antibody chains. The antibody chains included one heavy and one light chain that binds to the tumor antigen and one heavy and one light chain that binds to CD3. The combinatorics of this approach afforded poor yields and are further associated with issues including generation of contaminants (e.g., a potential bivalent anti-CD3 contaminant). Multiple designs have been proposed to address the unmet need of producing pure and high-yielding bispecific T cell engagers. Disclosed herein, inter alia, are solutions to these and other problems in the art.

### BRIEF SUMMARY OF THE INVENTION

In an aspect is provided a peptide including: (i) a first anti-CD3 dimerizing domain bound to a CD38 binding domain through a first chemical linker; and (ii) a second anti-CD3 dimerizing domain bound to the CD38 binding domain through a second chemical linker; wherein the first anti-CD3 dimerizing domain is capable of non-covalently binding to the second anti-CD3 domain to form an anti-CD3 binding domain.

In an aspect is provided an isolated nucleic acid encoding a peptide provided herein, including embodiments thereof.

In another aspect is provided a T lymphocyte including an expression vector provided herein, including embodiments thereof.

In an aspect a method of treating cancer in a subject in need thereof is provided, the method including administering to a subject a therapeutically effective amount of a peptide provided herein, including embodiments thereof.

In an aspect is provided a pharmaceutical composition including a therapeutically effective amount of a peptide provided herein, including embodiments thereof and a pharmaceutically acceptable excipient.

### BRIEF DESCRIPTION OF THE DRAWINGS

FIG. 1. Illustrates a 3-dimensional structure cartoon of a single-chain anti-CD38/anti-CD3 bispecific T cell engager following cleavage of the protease substrate. The right panel shows dimerization of the two anti-CD3 single chains, and the left panel shows binding of the anti-CD38 nanobody to its target.

FIG. 2. Illustrates a representative image of non-reducing and reducing gels showing analysis of the purified bispecific compound.

FIGS. 3A and 3B. Illustrates surface plasmon resonance (SPR) curves showing binding of compounds. FIG. 3A shows binding of bispecific compounds 3-38N001 (SEQ ID NO: 16), 3-38BN002 (SEQ ID NO:17) and 38BN003 (SEQ ID NO:18) including a protease cleave site and bispecific compounds 3-38N004 (SEQ ID NO:19), 3-38BN005 (SEQ ID NO:20) and 38BN006 (SEQ ID NO:21) without a cleavage site to CD38. FIG. 3B shows positive and negative controls Daratumumab and Trastuzumab binding to CD38.

FIGS. 4A and 4B. Illustrates SPR curves showing binding of compounds. FIG. 4A shows binding of bispecific compounds 3-38N001, 3-38BN002 and 38BN003 including a protease cleave site and bispecific compounds 3-38N004, 3-38BN005 and 38BN006 without a cleavage site to CD3. FIG. 4B shows positive and negative controls anti-CD3 Fab and Trastuzumab Fab binding to CD3.

FIG. 5A. Illustrates a graph depicting the effect of bispecific compound CD3-CD38 on antibody dependent cytotoxicity. The bispecific compound induces higher levels of antibody dependent cytotoxicity in the Acute Myelogenous Leukemia (AML) cell line Nomo1 as compared to the AML SKM1 cell line as measured by percentage cell lysis.

FIGS. 5B and 5C. FIG. 5B illustrates histograms showing the effect of bispecific compound CD3-CD38 on binding. The bispecific compounds display increased binding to the Nomo1 cell line as compared to the SKM1 cell line. FIG. 5C illustrates a histogram comparing the binding of the CD3-CD38 bispecific compound to Nomo1 and SKM1 cells and illustrating that the bispecific compound binds to Nomo1 cells to a higher degree than SKM1 cells.

FIG. 6A. Illustrates a flow cytometry graph analysis showing binding of CD3-CD38 bispecific compounds 38BN-001, 38BN-002, 38BN-003, 38BN-004, 38BN-005 or 38BN-001 to peripheral blood mononuclear cells (PBMC). The PMBC were treated for 1 hour with either 10 μg/mL of a bispecific compound or 10 μg/mL of Daratumumab.

FIG. 6B. Illustrates histograms showing binding of CD3-CD38 bispecific compounds to PBMC cells.

FIGS. 7A and 7B. Illustrate graphs depicting the effect of the bispecific compound CD3-CD38 on ADCC cell line. The bispecific compounds induce ADCC in the multiple myeloma cell line MM1S, which is CD38+. FIG. 7A shows HD41 PBMCs as used effector cells. FIG. 7B shows HD48 PBMCs as used effector cells.

FIG. 7C. Illustrates histograms illustrating that the CD3-CD38 bispecific compounds bind to MM1S target cells. A non-binding IgG antibody was used as a control.

FIG. 8A-8N. Illustrate CD38-CD3 (BN004) mediated killing of cancer cell lines is directly correlated with CD38 expression. FIG. 8A: Target cells (T) such as THP1 (CD38^{Hi} AML cell line), or MM.1S cells (CD38^{Hi} multiple myeloma cell line), or MV-4; 11 (CD38^{Low/Null} AML cell line) were co-cultured with healthy donor T-cells (effectors, E) in presence of control IgG or BN004 (CD38-CD3 BIONICS) overnight at different concentrations as reported and E:T ratio of 1:1. Cancer cell killing was assessed by the % of 7-ADD positive cancer cells by flow cytometry analysis; FIG. 8B: CD38 expression on the surface of the three different cell lines was assessed by flow cytometry analysis confirming high CD38 expression in THP-1 and MM.1S cell lines compared to MV-4; 11 cells; FIGS. 8C-8H: BN004 specific killing activity and T cell activation was assessed when AML (THP-1), MM (MM.1S) and T-ALL (Molt-4) CD38+ cell lines were cocultured either with BN004 or with a CD38-CD3 non-binding BIONIC (single mutation in CD38, BN023, or carrying a non-specific binding region, CD3-IL-1RAP) or with control IgG (FIGS. 8C, 8F and 8I). Early (CD69+) (FIGS. 8D, 8G and 8J) and late (CD25+) (FIGS. 8E, 8H and 8K) CD8+ T cell activation was observed only in presence of BN004 in all cell lines we tested following overnight incubation E:T ratio of 1:1 at different concentration. Killing activity data were repeated using at least T cells isolated from n=3 different healthy donors and repeated in two or three technical replicates for each donor. FIG. 8L: CD38+HL60 AML cell lines wild type (WT) or CD38 knockout (CRISPER, KO) were treated with 1.0 ng/ml of control IgG or BN004 or BN023 and incubated overnight at the E:T ratio of 1:1. Next day, cancer cell killing by 7-ADD and T cell late activation (CD25+) was assessed by flow cytometry analysis, showing significant cell killing only when T cell were incubated with CD38+HL60 WT; FIG. 8N: Peripheral mononuclear cells from n=3 healthy donors were incubated with 1 ng/ml of BN004 or BN023 or control IgG in presence (T+AML) or absence (T alone) of AML cells (THP-1). Supernatant was collected after 16 hours and analyzed by cytokine array. Data show that BN004 induced the release of T cell inflammatory cytokines including IFN-γ, TNF-alpha and IL-2. The experiment was repeated in n=3 biological replicates and in at least n=2 technical replicates.

FIG. 9A-9I. Illustrate CD38-CD3 (BN004) mediated killing of primary AML cancer cells by redirecting patient T cells against their matching cancer cells. FIG. 9A: The peripheral blood (n=5) and the bone marrow (n=2) samples obtained from AML patients were treated with different BN004 or control IgG concentrations (0.1, 10 and 100 ng/ml) and markers of early (CD69) and late (CD25) T cell activation were assessed between 48 and 96 hrs after treatment; FIG. 9B: Representative flow cytometry analysis showing decrease of the CD45dim (AML) population in primary bone marrow mononuclear cells of a relapsing AML patient treated with BN004 or control IgG at the indicated concentrations; FIG. 9C: Violin plot showing significant dose dependent reduction of primary AML cells upon BN004 treatment compared to control IgG; FIGS. 9D-9G: Mass cytometry analysis of the total bone marrow cellular population (FIG. 8D) and total peripheral blood population (FIG. 9F) obtained from two relapsing AML patients treated for 48 hours with 1 ng/ml of BN004 or CD38 mutated non-binding BIONICS (BN023) or control IgG showing how the AML population (highlighted in red) is depleted while the T cell populations (highlighted in brown and blue) is expanding. CD8+ T cell population is also expanded by BN004 as shown in total T cell counts (FIGS. 9E and 9G); FIG. 9H: tSNE plot highlighting the different AML populations which were specifically depleted upon BN004 treatment. Elimination of the CD34+CD38-leukemia stem cell population was also observed with BN004 compared to BN0023, control IgG and untreated cellular population.

FIGS. 10A-10E. Illustrate CD38-CD3 BIONICS targets leukemia stem cells (LSCs). FIG. 10A: CD38-CD3 BIONIC (BN004) redirects T-cells to eliminate autologous AML LSCs, while sparing T-cells from eliminating healthy stem cells in healthy bone marrow. MNCs from healthy donor and AML patients were treated with 1.0 ng/ml of control-IgG and BN004 and plated for 14 days in MegaCult™ GF H4034 medium for enumeration of hematopoietic progenitor cells in colony forming unit (CFU) assay; FIG. 10B: Graph showing that CD38-CD3 BIONICS significantly eradicates LSCs; FIGS. 10C-10D: Specifically, 25 mice were engrafted with 1×10\hat{ }6 THP-1 Gfp+/Luciferase positive cells and after 18 days equally engrafted mice were divided in two treatment groups. 12 mice in the control group and 8 mice in the BN004 treatment arm. Mice were treated with 2.5 mg/kg of BN004 or control IgG in combination with 3×10\hat{ }6 total T cells for six weeks; FIG. 10C: Luminescence signal at pre-treatment (day 18^{th}) and during treatment; FIG. 10D: Decrease in Luminescence signal in mice treated with BN004; FIG. 10E: illustrates survival curve showing increase % of survival in mice treated with BN004. BN004 completely eliminated AML engraftment in 3 out of 8 treated mice. One mouse out of 3 was then scarified at day 80 for minimal residual disease assessment and the other two mice were followed for more than 120 days and no signs of leukemia were detected and they are still alive after 160 days.

FIGS. 11A-11J. CD38-CD3 BIONICS kills aggressive cancer cells in in vivo studies. FIG. 11A: Luminescence image of NSG mice intravenously injected with 5×10\hat{ }6 MM.1SGfp/Luc+MM cells and treated with T cells in combination with BN004 or control IgG. After two weeks when mice showed diffuse bone marrow myeloma infiltration, mice were randomly divided and treated with either control IgG, or BN004 (2.5 mg/kg) in combination with 3×10\hat{ }6 total T cells once a week for two weeks; FIG. 11B: Graph showing that only two treatments of CD38-CD3 BIONICS significantly increased survival and reduced overall tumor burden; FIG. 11C: Representative flow cytometry analysis showing lower AML patient derived engraftment in the bone marrow (BM) and spleen of NSG mice treated with BN004 (n=5) compared to animal treated with the non-binding CD38 mutant BIONIC (BN023) (n=5) or control IgG (n=5). Specifically, 1×10\hat{ }6 of total BM cells obtained from an AML relapsing patient were intravenously injected (IV) in irradiated (dose 150 centigray (cGy) for 150 second) NSG mice. After engraftment (7 days) mice were treated with 2.5 mg/kg of control IgG, or BN004 or BN023 in combination with 3×10\hat{ }6 total T cells once a week for three weeks. Mice were then humanely scarified and AML engraftment in the BM and spleen were checked by glow cytometry analysis; FIG. 11D: Graphs showing the significant decrease of AML cell engraftment in mice treated with BN004 over BN023 or control IgG; FIG. 11E: Representative flow cytometry analysis showing lower T-ALL patient derived engraftment in the peripheral blood of mice treated with BN004 (2.5 mg/kg) in combination with 3×10\hat{ }6 T cells compared to untreated engrafted animals; FIG. 11F: Bar graph showing the significant difference in engraftment between BN004 and untreated animals; FIG. 11G: Treatment schedule of mice injected with highly aggressive and bone marrow metastatic Gfp+/Luc+AML cell line (U937) in which we compared the in vivo activity of the CD38-CD3 BIONICS containing an anti-CD3 sequence (BN004) with the CD38-CD3 BIONIC containing the City of Hope generated anti-CD3 sequence (BN008), with the single non-binding CD38 mutation BIONIC (BN023) with the control IgG treated mice; Specifically, 20 mice were engrafted with 1×10\hat{ }6 U937 Gfp+/Luciferase positive cells and after 4 days equally engrafted mice were divided in four treatment groups. 5 for group. Mice were treated with 2.5 mg/kg of BN004 or control IgG in combination with 3×10\hat{ }6 total T cells once a week for 3 weeks; FIG. 11H: Luminescence signal of each treatment group after 6 and 13 days from the first treatment (day 10 and 17); FIG. 11I: Bar graphs showing significant decrease in luminescence signal in mice treated with BN004 and BN08 versus BN0023 and control IgG; FIG. 11J: Survival curve showing increase % of survival in mice treated with BN004 and BN008 compared to BN023 and IgG treated mice.

FIGS. 12A-12K. Illustrate BN004 through T cell IFN-γ release induces differentiation of leukemic stem cells (LSCs) (CD34+CD38−) into CD38+ cells and suppresses LSCs stemness. FIG. 12A: CD38-CD3 bionic re-directs T-cells to secrete IFN-γ against CD38+AML cells and THP1 CD38Hi AML cell line (target, T) was co-cultured with healthy donor T-cells (effectors, E) in presence of control IgG, BN004 (CD38-CD3), BN023 (CD38-Mutant) overnight at concentration of 1.0 ng/ml and E:T ratio of 1:1. T-cells alone were as well treated with 1.0 ng/ml of control-IgG, BN004, and BN023. Following overnight incubation, supernatants were collected and IFN-γ concentration was determined with Human 10-plex cytokine immunoassay. Data are reported as mean±SD of three healthy donors; ****p<0.0001; n.s. not significant. FIG. 12B: AML patient peripheral blood samples were treated overnight with 1.0 ng/ml of control IgG and BN004. Next day, RNA was extracted from treated cells and subjected to qRT-PCR using IFN-γ and GAPDH TaqMan probes; FIG. 12C: CD38-CD3 bionic induces CD38 expression in AML CD45Dim CD34+CD38-leukemic stem cell (LSC) and AML CD45Dim CD34+CD38+ blast populations (n=3 patient samples); FIG. 12D: Mononuclear cells (MNCs) were isolated from a leukopharesis sample obtained from an AML patient and CD34+AML cells were treated at different time points (24, 48 and 72 hours) either with BIONICS (BN004, BN023), or control IgG or IFN-γ as indicated in the figures. Either BN004 or IFN-γ treatments induced increased CD38 surface expression in CD34+CD38negative LSC population at the different time points; FIGS. 12E-12G: Colony forming assays using AML bone marrow samples (n=3) treated with BN004 or BN023 or control IgG (1 ng/ml) or IFN-γ (10 ng/ml), showing significant colony reduction when BM samples were treated with either BN004 (E) or IFN-γ (FIGS. 12F and 12G) compared to the respective controls; FIGS. 12H and 12I: Colony forming assays using healthy bone marrow samples treated with IFN-γ (10 ng/ml), showing no significance difference compared to the untreated control; FIG. 12E or IFN-γ (FIGS. 12F and 12G) compared to the respective controls; FIGS. 12J and 12K) Flow cytometry analysis (FIG. 12J) and respective graph bar (FIG. 12K) showing that IFN-γ blocking antibody can revert CD38 upregulation on the surface of AML cells (THP-1) treated with BN004 in presence of T cells.

FIGS. 13A-13F. Illustrate Exogenous IFN-γ induces CD38 in AML not in healthy donor bone marrow. AML (FIG. 13A) and healthy (FIG. 13B) bone marrow mononuclear cells (BM-MNCs) were treated with IFN-γ at 100 ng/ml for 72 hours. At 72 hours, cells were collected and subjected to surface staining of CD45, CD34, and CD38 and analyzed by flow cytometry; FIGS. 13C and 13D: Total BM-MNCs from AML patient (FIG. 13C) or healthy donor (FIG. 13D) were treated at different IFN-γ concentrations as indicated and variation of CD38 expression in the CD45dim gaited population was observed. Dose dependent increase in CD38 expression expressed in MFI was observed in the AML BM (bar graph in FIG. 13C) but not in the healthy donor (FIG. 13D) treated in the same experimental conditions (72 hours). FIGS. 13E and 13F: T-cells were isolated from healthy donor PBMCs and treated with different concentration of IFN-γ for 72 hours. At 72 hours, T-cells were subjected to CD3 and CD38 surface staining and analyzed on flow cytometer.

FIG. 14. is a graphical representation of the therapeutic rationale to target CD38 LSCs with T cell engagers against CD38 such as CD38-CD3 bionics eradicate AML by targeting leukaemia stem cells through the IFN-γ/CD38 regulatory loop.

FIG. 15. Is a heat map showing cell types in an population of AML cells.

FIGS. 16A-16B. Illustrate a rationale for targeting CD38 in AML. CD38 is highly expressed in AML patients. Compared to other tumor types, CD38 expression is particularly high in AML cancer cells (FIG. 16A). In the TCGA AML cohort, which include favourable, intermediate and poor cytogenetic risk groups, samples were obtained from 173 AML patients. Transcript levels show that CD38 expression correlates with CD34, one of the most abundant AML markers. The linear correlation value between CD38 and CD34 is R=0.285, with a p value=1.47e-04 (FIG. 16B).

FIGS. 17A-17B. A BiTE can be designed to adopt an optimized geometry for ADCC activity. A schematic showing the cancer cell membrane and T cell membrane with the TCR/CDR complex in between shows that a distance of about 130 Å between membranes is ideal. (FIG. 17A). Results from a T cell activation assay indicate geometries as shown in FIG. 17A that result in good ADCC, minor ADCC, and no ADCC (FIG. 17B).

FIG. 18. Is a space filling model of a CD38-CD3 bionic (biologics nested inside chains). CD38-CD3 bionics are able to bring in close distance T cells and CD38+ cancer cells, and therefore can be a therapeutic strategy for MM, AML and T-ALL. The bionics are efficient single peptide chains that can be made in high yields (e.g. 1-3 g/L transiently) and have high stability (e.g. 5° C. to 10° C. increase in melting temp compared to Fab). The bionics display no chain pairing issues, and are compatible with Fc and other lifetime extension additions.

FIGS. 19A-19D. Illustrate that bionics have strong affinity and anti-cancer activity. Sensograms show that the CD38-CD3-bionic shows strong binding to CD38 (FIG. 19A) and CD3 (FIG. 19B), respective antigens immobilized on an SPR chip. Flow cytometry data illustrates that the CD38-CD3-bionic engages CD38 and CD3 in primary patient samples, as compared to a control IgG (FIG. 19C). Target cells (T) such as THP1 (CD38^{Hi} AML cell line), or MM.1S cells (CD38^{Hi} multiple myeloma cell line), or MV-4; 11 (CD38^{Low/Null} AML cell line) were co-cultured with healthy donor T-cells (effectors, E) in presence of control IgG or BN004 (CD38-CD3 BIONICS) overnight at different concentrations as reported and E:T ratio of 1:1. T cell activation was assessed by the % of CD25 positive cancer cells by flow cytometry analysis (FIG. 19C).

FIGS. 20A-20D. Illustrate that the CD38-CD3 bionic activates AML patient T cells ex-vivo. The CD38-CD3 bionic was incubated with samples from AML patients. Samples tested include peripheral blood of relapsing patients (n=3) treated with bionic for 48 hours, peripheral blood of newly diagnosed patients (n=2) treated with bionic for 48 hours, and bone marrow of AML patients (n=2) treated with bionic for 96 hours. Flow cytometry data shows that the bionic induces CD69 as early activation marker in CD4+ (FIGS. 20A and 20C) and CD8+ (FIGS. 20B and 20D) T cells obtained from AML patients within 48 hrs.

FIGS. 21A-21D. show that CD38-CD3 BIONIC induces CD25, a marker associated with T cell “activation-induced” cancer cell death in both CD4+ (FIGS. 21A and 21C) and CD8+ (FIGS. 21B and 21D) T cells isolated from AML patients. Samples tested include peripheral blood of relapsing patients (n=3) treated with bionic for 48 hours, peripheral blood of newly diagnosed patients (n=2) treated with bionic for 48 hours, and bone marrow of AML patients (n=2) treated with bionic for 96 hours.

FIG. 22. Illustrates that CD38-CD3 bionics redirect AML T cells to kill autologous cancer cells. Using peripheral blood samples obtained from newly diagnosed or relapsing AML patients the ability of the Bionics to redirect patient T cells against their matching cancer cells were assessed. The samples include peripheral blood of relapsing AML Pts (n=2) CD45dimCD34+CD38+CD33+ blasts (n=2) treated with bionic for 5 days, peripheral blood of newly diagnosed AML patients CD45dimCD34neg.CD38+CD33+ (n=1) treated with bionic for 5 days, and peripheral blood of relapse AML Pt CD45dimCD34neg.CD38+CD33+ blasts (treatment days=5).

FIG. 23. Shows representative images from a colony formation assay showing CD38-CD3 BIONIC activates T cells against autologous leukemia stem cells.

FIG. 24. Illustrates a treatment schedule of mice (top panel) and shows that CD38-CD3 bionics have strong preclinical activity in AML models. Luminescence signal is shown at the indicated days of treatment (bottom panel).

FIG. 25. Illustrates a treatment schedule of mice for the study shown in FIG. 11D.

### DETAILED DESCRIPTION

### Definitions

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

A “chemical linker,” as provided herein, is a covalent linker, a non-covalent linker, a peptide or peptidyl linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof.

The chemical linker as provided herein may be a bond, —O—, —S—, —C(O)—, —C(O)O—, —C(O)NH—, —S(O)₂NH—, —NH—, —NHC(O)NH—, substituted (e.g., substituted with a substituent group, a size-limited substituent or a lower substituent group) or unsubstituted alkylene, substituted (e.g., substituted with a substituent group, a size-limited substituent or a lower substituent group) or unsubstituted heteroalkylene, substituted (e.g., substituted with a substituent group, a size-limited substituent or a lower substituent group) or unsubstituted cycloalkylene, substituted (e.g., substituted with a substituent group, a size-limited substituent or a lower substituent group) or unsubstituted heterocycloalkylene, substituted (e.g., substituted with a substituent group, a size-limited substituent or a lower substituent group) or unsubstituted arylene or substituted (e.g., substituted with a substituent group, a size-limited substituent or a lower substituent group) or unsubstituted heteroarylene.

The chemical linker as provided herein may be a bond, —O—, —S—, —C(O)—, —C(O)O—, —C(O)NH—, —S(O)₂NH—, —NH—, —NHC(O)NH—, substituted or unsubstituted (e.g., C₁-C₂₀, C₁-C₁₀, C₁-C₅) alkylene, substituted or unsubstituted (e.g., 2 to 20 membered, 2 to 10 membered, 2 to 5 membered) heteroalkylene, substituted or unsubstituted (e.g., C₃-C₈, C₃-C₆, C₃-C₅) cycloalkylene, substituted or unsubstituted (e.g., 3 to 8 membered, 3 to 6 membered, 3 to 5 membered) heterocycloalkylene, substituted or unsubstituted (e.g., C₆-C₁₀, C₆-C₈, C₆-C₅) arylene or substituted or unsubstituted (e.g., 5 to 10 membered, 5 to 8 membered, 5 to 6 membered,) heteroarylene.

In embodiments, the chemical linker is a covalent linker. In embodiments, the chemical linker is a hydrocarbon linker. In embodiments, the chemical linker is a cleavable peptide linker.

Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition).

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

A “labeled protein or polypeptide” is one that is bound, either covalently, through a linker or a chemical bond, or noncovalently, through ionic, van der Waals, electrostatic, or hydrogen bonds to a label such that the presence of the labeled protein or polypeptide may be detected by detecting the presence of the label bound to the labeled protein or polypeptide. Alternatively, methods using high affinity interactions may achieve the same results where one of a pair of binding partners binds to the other, e.g., biotin, streptavidin.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“CD3” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex. In embodiments, the CD3 protein is substantially identical to the protein identified by the UniProt reference number P04234 or a variant or homolog having substantial identity thereto. In embodiments, the CD3 protein is substantially identical to the protein identified by the UniProt reference number P09693 or a variant or homolog having substantial identity thereto. In embodiments, the CD3 protein is substantially identical to the protein identified by the UniProt reference number P07766 or a variant or homolog having substantial identity thereto.

“CD38” as referred to herein includes any of the recombinant or naturally-occurring forms of the Cluster of Differentiation 38 (CD38) protein, also known as ADP-ribosyl cyclase/cyclic ADP-ribose hydrolase 1, 2′-phospho-ADP-ribosyl cyclase, 2′-phospho-cyclic-ADP-ribose transferase, ADP-ribosyl cyclase 1, or variants or homologs thereof that maintain CD38 activity (e.g. within at least 50%, 80%, 90%, 95%, 96%, 97%, 98%, 99% or 100% activity compared to CD38). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD38 protein. In embodiments, the CD38 protein is substantially identical to the protein identified by the UniProt reference number P28907 or a variant or homolog having substantial identity thereto.

“Her2” as referred to herein includes any of the recombinant or naturally-occurring forms of the human epidermal growth factor receptor 2 protein, also known as receptor tyrosine-protein kinase erbB-2, or variants or homologs thereof that maintain Her2 activity (e.g. within at least 50%, 80%, 90%, 95%, 96%, 97%, 98%, 99% or 100% activity compared to Her2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Her2 protein. In embodiments, the Her2 protein is substantially identical to the protein identified by the UniProt reference number P04626 or a variant or homolog having substantial identity thereto.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region; also referred to herein as “Fc domain”) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. In embodiments, the Fc region includes a constant heavy chain domain 3 (CH3 domain) and a constant heavy chain domain 2 (CH2 domain).

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

An “antibody variant” as provided herein refers to a polypeptide capable of binding to an antigen and including one or more structural domains of an antibody or fragment thereof. Non-limiting examples of antibody variants include single-domain antibodies or nanobodies, affibodies (polypeptides smaller than monoclonal antibodies (e.g., about 6kDA) and capable of binding antigens with high affinity and imitating monoclonal antibodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “nanobody” or “single domain antibody” as described herein is commonly well known in the art and refers to an antibody fragment consisting of a single monomeric variable antibody domain (e.g., a VH or a VL domain). Like a whole antibody, it is able to bind selectively to a specific antigen. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

An “affibody” as described herein is commonly well known in the art and refers to small, robust proteins engineered to bind to a large number of target proteins or peptides with high affinity, by imitating monoclonal antibodies. Affibodies are therefore a member of the family of antibody mimetics. In embodiments, an affibody is a molecule including of three alpha helices with about 58 amino acids and a molar mass of about 6 kDa.

A “single domain antibody” as provided herein refers to an antibody fragment including a single monomeric variable antibody domain (e.g., a VH or a VL domain). Like a whole antibody, a single domain antibody is able to bind selectively to a specific antigen. The molecular weight of a single domain antibody is 12-15 kDa, single domain antibody. In embodiments, a single domain antibody is a variable heavy chain domain. In embodiments, a single domain antibody includes a variable heavy chain domain. In embodiments, a single domain antibody is a variable light chain domain. In embodiments, a single domain antibody includes a variable light chain domain. Non-limiting examples of single domain antibodies include camelid-derived VHH fragments and VNAR (variable immunoglobulin new antigen receptor) fragments. In embodiments, the single-domain antibody is a peptide domain of about 110 amino acids.

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a ligand binding domain (e.g., receptor or antibody, antibody variant, antibody region or fragment thereof).

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

The term “plasmid,” “expression vector,” or “viral vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. Suitable viral vectors contemplated herein include, for example, lentiviral vectors and onco-retroviral vectors.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer) means that the disease is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example, certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

In embodiments, the methods provided herein further include administering to the subject an additional therapeutic agent. As described above, a therapeutic agent is a composition useful in treating or preventing a disease such as cancer. In embodiments, the additional therapeutic agent is an anti-cancer agent.

The terms “anti-cancer agent” and “anticancer agent” are used in accordance with their plain ordinary meaning and refer to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rIL.sub.2), interferon alfa-2a; interferon alfa-2b; interferon alfa-n1; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39·HCl), AC-7700 (Ajinomoto, i.e. AVE-8062, AVE-8062A, CS-39-L-Ser·HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111 In, 90Y, or 131I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Peptide Compounds

Provided herein are, inter alia, peptide compositions having bi-specific binding capabilities useful for therapeutic and diagnostic purposes. The peptide compositions provided herein are single-chain polypeptides including an anti-CD3 binding domain and a CD38 binding domain able to target (bind) CD3 and CD38 at the same time. The peptide compositions provided herein can be produced at very high yields and are therefore easy to manufacture. The peptide compositions provided herein are, inter alia, therapeutically useful for the simultaneous targeting of CD3 expressed, for example, on an immune cell (e.g., Nk cells, T cells), and CD38 expressed, for example, on a cancer cell, thereby sequestering these antigens. Through this sequestering of antigens, the immune cell and the cancer cell are brought into close proximity to provide for efficient targeted cell killing of the cancer cell through the immune cell. The peptide compositions provided herein may include, without limitation, domains of an antibody, antibody variant or fragments thereof (e.g., single chain antibodies, nanobodies, affybodies, Fabs). The peptide compositions provided herein are surprisingly effective compared to traditional bispecific antibodies.

The chemical linkers (e.g., first and/or second chemical linker) included in the peptide compositions provided herein may be non-cleavable and based on their respective length (amino acid length across the longest distance) the peptide is sterically configured such that the anti-CD3 binding domain and the CD38 binding domain are accessible for binding their respective ligands. Alternatively, the chemical linkers (e.g., first and/or second chemical linker) included in the peptide compositions provided herein may be cleavable and thereby conveying disease site specificity to the compositions provided herein. For example, the first and/or second chemical linker may be a cleavable linker including a cleavage site recognized by a tumor-specific protease. In the absence of a tumor-specific protease the first and/or second chemical linker is not cleaved and the peptide composition is in a sterically occluded conformation, wherein the first ligand binding domain and/or the second ligand binding domain do not bind their corresponding ligand. In the presence of a tumor-specific protease the first and/or second chemical linker is cleaved and the peptide composition forms a sterically open conformation, wherein the anti CD3 binding domain and/or the CD38 binding domain are capable of binding their corresponding ligand. Thus, additional functionality (e.g., tumor-specific activation) can be included in the peptide compositions through steric hindrance or masking of one or more ligand binding domains.

The peptide compositions provided herein are surprisingly stable and exhibit increased affinity to CD3 and CD38, respectively. Therefore, the compositions and methods provided herein address the need in the art for high yield production of bispecific therapeutics (e.g., bispecific antigen binders) that are highly effective and specific exhibiting minimal adverse effects. The peptides provided herein including embodiments thereof include an anti-CD3 and a CD38 binding domain connected through a linker and both binding domains are able to bind to their respective ligand without cleavage of the linker. The peptides provided herein that do not require cleavage of the linker (e.g., first or second chemical linker) for the anti-CD3 and the CD38 binding domain to bind their respective ligands, are also referred to herein as “bionics” or “bionic molecules.”

The peptides may include a cleavable linker (first or second chemical linker) and only upon cleavage of the first or second chemical linker are the anti CD3 and/or CD38 binding domain able to bind to their corresponding ligand/binding partner. Peptides including a cleavable linker and a first ligand binding domain which is occluded unless the linker is cleaved are also referred to herein as “switchblade” or “switchblade molecule.”

In an aspect is provided a peptide including: (i) a first anti-CD3 dimerizing domain bound to a CD38 binding domain through a first chemical linker; and (ii) a second anti-CD3 dimerizing domain bound to the CD38 binding domain through a second chemical linker; wherein the first anti-CD3 dimerizing domain is capable of non-covalently binding to the second anti-CD3 domain to form an anti-CD3 binding domain.

A “binding domain” as provided herein refers to a peptide domain capable of selectively binding to a target ligand (e.g., CD3, CD38 or fragments thereof). A binding domain may covalently or non-covalently bind to a target ligand. Non-limiting examples of binding domains include single chain antibodies, antibody variants or fragments thereof, antibodies or fragments thereof, an antibody Fc region or fragments thereof. In embodiments, the binding domain is a Fab. In embodiments, the binding domain is a single domain antibody (sdAb). In embodiments, the binding domain is a nanobody.

A “dimerzing domain” as referred to herein is a polypeptide including an antibody VH domain or fragment thereof bound (covalently and/or non-covalently) to an antibody CH domain (e.g., CH1 domain) or fragment thereof. Upon binding of two dimerizing domains a Fab domain is formed. Thus, Fab domain may include a first dimerzing domain (e.g., anti-CD3 dimerizing domain) non-covalently or covalently bound to a second dimerizing domain (e.g., anti-CD3 dimerizing domain). In embodiments, the CH domain (e.g., CH1 domain) of the first anti-CD3 dimerizing domain is non-covalently bound to the CH domain (e.g., CHI domain) of the second anti-CD3 dimerizing domain. In embodiments, the VH domain of the first anti-CD3 dimerizing domain is covalently bound to the VH domain of the second anti-CD3 dimerizing domain. In embodiments, the CH domain (e.g., CH1 domain) of the first anti-CD3 dimerizing domain is bound to the CH domain (e.g., CH1 domain) of the second anti-CD3 dimerizing domain through a disulfide linkage. In embodiments, the anti-CD3 dimerizing domain includes a VH domain and a CH domain (e.g., CH1 domain). In embodiments, the anti-CD3 dimerizing domain includes from the N-terminus to the C-terminus a VH domain and a CH domain (e.g., CH1 domain).

The CD38 binding domain may be a single-domain antibody domain. The anti-CD3 binding domain may be a protein domain including two protein dimerizing domains (e.g., a first and a second anti-CD3 dimerizing domain). The first anti-CD3 dimerzing domain and the second anti-CD3 dimerizing domain may be covalently and/or non-covalently bound to each other. Thus, in embodiments, the first anti-CD3 dimerizing domain is bound to the second anti-CD3 dimerizing domain. In embodiments, the peptide further includes a covalent bond connecting the first anti-CD3 dimerizing domain and the second anti-CD3 dimerizing domain. In embodiments, the first anti-CD3 dimerizing domain is bound to the second anti-CD3 dimerizing domain. In embodiments, the covalent bond is a disulfide bond. In embodiments, the anti-CD3 binding domain is a Fab domain.

In embodiments, the first anti-CD3 dimerizing domain includes a variable light chain domain. In embodiments, the first anti-CD3 dimerizing domain includes a constant light chain domain. In embodiments, the variable light chain domain is bound to the CD38 binding domain through the constant light chain domain. In embodiments, the variable light chain domain is bound to the CD38 binding domain through the first chemical linker. In embodiments, the first anti-CD3 dimerizing domain includes an antibody light chain. In embodiments, the first anti-CD3 dimerizing domain is an antibody light chain. In embodiments, the second anti-CD3 dimerizing domain includes a variable heavy chain domain. In embodiments, the second anti-CD3 dimerizing domain includes a constant heavy chain domain. In embodiments, the constant heavy chain domain is bound to the CD38 binding domain through the variable heavy chain domain. In embodiments, the variable heavy chain domain is bound to the CD38 binding domain through the second chemical linker. In embodiments, the second anti-CD3 dimerizing domain includes an antibody heavy chain. In embodiments, the second anti-CD3 dimerizing domain is an antibody heavy chain.

In embodiments, the first anti-CD3 dimerizing domain includes a variable heavy chain domain. In embodiments, the first anti-CD3 dimerizing domain includes a constant heavy chain domain. In embodiments, the variable heavy chain domain is bound to the CD38 binding domain through the constant heavy chain domain. In embodiments, the variable heavy chain domain is bound to the CD38 binding domain through the first chemical linker. In embodiments, the first anti-CD3 dimerizing domain includes an antibody heavy chain. In embodiments, the first anti-CD3 dimerizing domain is an antibody heavy chain. In embodiments, the second anti-CD3 dimerizing domain includes a variable light chain domain. In embodiments, the second anti-CD3 dimerizing domain includes a constant light chain domain. In embodiments, the constant light chain domain is bound to the CD38 binding domain through the variable light chain domain. In embodiments, the variable light chain domain is bound to the CD38 binding domain through the second chemical linker. In embodiments, the second anti-CD3 dimerizing domain includes an antibody light chain. In embodiments, the second anti-CD3 dimerizing domain is an antibody light chain. In embodiments, the anti-CD3 binding domain is a Fab domain.

A “variable light chain (VL) domain” as provided herein refers to the variable region of the light chain of an antibody, an antibody variant or fragment thereof. Likewise, the “variable heavy chain (VH) domain” as provided herein refers to the variable region of the heavy chain of an antibody, an antibody variant or fragment thereof. As described above, the variable light chain domain and the variable heavy chain domain together form the paratope, which binds an antigen (epitope). The paratope or antigen-binding site is formed at the N-terminus of an antibody, an antibody variant or fragment thereof. In embodiments, the variable light chain (VL) domain includes CDR L1, CDR L2, CDR L3 and FR L1, FR L2, FR L3 and FR L4 (framework regions) of an antibody light chain. In embodiments, the variable heavy chain (VH) domain includes CDR H1, CDR H2, CDR H3 and FR H1, FR H2, FR H3 and FR H4 (framework regions) of an antibody heavy chain.

The terms “CDR L1”, “CDR L2” and “CDR L3” as provided herein refer to the complementarity determining regions (CDR) 1, 2, and 3 of the variable light (L) chain of an antibody or fragment thereof. In embodiments, the variable light chain includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody or fragment thereof. In embodiments, the variable heavy chain includes in N-terminal to C-terminal direction a CDR H1, a CDR H2 and a CDR H3.

“Framework regions” (FRs) are variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of the variable region of the heavy chain are further referred to herein as HFR1, HFR2, HFR3 and HFR4, respectively, wherein HFR1 corresponds to FR 1 of VH, HFR 2 corresponds to FR 2 of VH, HFR 3 corresponds to FR 3 of VH and HFR 4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL. Likewise, the FRs of the variable region of the light chain are further referred to herein as LFR1, LFR2, LFR3 and LFR4, respectively, wherein LFR1 corresponds to FR 1 of VL, LFR 2 corresponds to FR 2 of VL, LFR 3 corresponds to FR 3 of VL and LFR 4 corresponds to FR 4 of VL.

In embodiments, the variable light chain (VL) domain and a constant light chain (CL) domain form part of an antibody light chain. In embodiments, the variable heavy chain (VH) domain and a constant heavy chain (CH1) domain form part of an antibody heavy chain. In embodiments, the variable heavy chain (VH) domain and one or more constant heavy chain (CH1, CH2, or CH3) domains form part of an antibody heavy chain. In embodiments, the variable light chain (VL) domain forms part of an antibody fragment. In embodiments, the variable heavy chain (VH) domain forms part of an antibody fragment. In embodiments, the variable light chain (VL) domain forms part of an antibody variant. In embodiments, the variable heavy chain (VH) domain forms part of an antibody variant. In embodiments, the variable light chain (VL) domain forms part of a Fab. In embodiments, the variable heavy chain (VH) domain forms part of a Fab. In embodiments, the variable light chain (VL) domain forms part of a scFv. In embodiments, the variable heavy chain (VH) domain forms part of a scFv.

In embodiments, the second anti-CD3 dimerizing domain includes a variable heavy chain domain. In embodiments, the second anti-CD3 dimerizing domain includes a constant heavy chain domain. In embodiments, the constant heavy chain domain is bound to the CD38 binding domain through the variable heavy chain domain. In embodiments, the constant heavy chain domain is bound to the CD38 binding domain through the second chemical linker. In embodiments, the second anti-CD3 dimerizing domain includes an antibody heavy chain. In embodiments, the second anti-CD3 dimerizing domain is an antibody heavy chain.

In embodiments, the first chemical linker is bound to the N-terminus of the CD38 binding domain and the second chemical linker is bound to the C-terminus of the CD38 binding domain. In embodiments, the first chemical linker is bound to the C-terminus of the CD38 binding domain and the second chemical linker is bound to the N-terminus of the CD38 binding domain.

In embodiments, the peptide includes from N terminus to C terminus a first anti-CD3 dimerizing domain, wherein the first anti-CD3 dimerizing domain is an antibody heavy chain, a first chemical linker, wherein the first chemical linker is a cleavable peptide linker, a CD38 binding domain, wherein the CD38 binding domain is a nanobody, a second chemical linker, wherein the second chemical linker is a cleavable peptide linker and a second anti-CD3 dimerizing domain, wherein the second anti-CD3 dimerizing domain is an antibody light chain.

In embodiments, the peptide includes from N terminus to C terminus a first anti-CD3 dimerizing domain, wherein the first anti-CD3 dimerizing domain is an antibody light chain, a first chemical linker, wherein the first chemical linker is a cleavable peptide linker, a CD38 binding domain, wherein the CD38 binding domain is a nanobody, a second chemical linker, wherein the second chemical linker is a cleavable peptide linker and a second anti-CD3 dimerizing domain, wherein the second anti-CD3 dimerizing domain is an antibody heavy chain.

In embodiments, the first anti-CD3 dimerizing domain is an antibody light chain including a CDR L1 as set forth in SEQ ID NO:1, a CDR L2 as set forth in SEQ ID NO:2 and a CDR L3 as set forth in SEQ ID NO:3. In embodiments, the first anti-CD3 dimerizing domain includes a CDR L1 as set forth in SEQ ID NO:1, a CDR L2 as set forth in SEQ ID NO:2 and a CDR L3 as set forth in SEQ ID NO:3. In embodiments, the antibody light chain includes the sequence of SEQ ID NO:7. In embodiments, the antibody light chain is the sequence of SEQ ID NO: 7. In embodiments, the first anti-CD3 dimerizing domain includes the sequence of SEQ ID NO: 7. In embodiments, the first anti-CD3 dimerizing domain is the sequence of SEQ ID NO:7.

In embodiments, the first anti-CD3 dimerizing domain is an antibody heavy chain including a CDR H1 as set forth in SEQ ID NO:4, a CDR H2 as set forth in SEQ ID NO:5, and a CDR H3 as set forth in SEQ ID NO:6. In embodiments, the first anti-CD3 dimerizing domain includes a CDR H1 as set forth in SEQ ID NO:4, a CDR H2 as set forth in SEQ ID NO:5, and a CDR H3 as set forth in SEQ ID NO:6. In embodiments, the antibody heavy chain includes the sequence of SEQ ID NO:8. In embodiments, the antibody heavy chain is the sequence of SEQ ID NO: 8. In embodiments, the first anti-CD3 dimerizing domain includes the sequence of SEQ ID NO: 8. In embodiments, the first anti-CD3 dimerizing domain is the sequence of SEQ ID NO: 8.

In embodiments, the second anti-CD3 dimerizing domain is an antibody heavy chain including a CDR H1 as set forth in SEQ ID NO:4, a CDR H2 as set forth in SEQ ID NO:5, and a CDR H3 as set forth in SEQ ID NO:6. In embodiments, the first anti-CD3 dimerizing domain includes a CDR H1 as set forth in SEQ ID NO:4, a CDR H2 as set forth in SEQ ID NO:5, and a CDR H3 as set forth in SEQ ID NO:6. In embodiments, the antibody heavy chain includes the sequence of SEQ ID NO:8. In embodiments, the antibody heavy chain is the sequence of SEQ ID NO: 8. In embodiments, the second anti-CD3 dimerizing domain includes the sequence of SEQ ID NO:8. In embodiments, the second anti-CD3 dimerizing domain is the sequence of SEQ ID NO: 8.

In embodiments, the second anti-CD3 dimerizing domain is an antibody light chain including a CDR L1 as set forth in SEQ ID NO:1, a CDR L2 as set forth in SEQ ID NO:2 and a CDR L3 as set forth in SEQ ID NO:3. In embodiments, the first anti-CD3 dimerizing domain includes a CDR L1 as set forth in SEQ ID NO:1, a CDR L2 as set forth in SEQ ID NO:2 and a CDR L3 as set forth in SEQ ID NO:3. In embodiments, the antibody light chain includes the sequence of SEQ ID NO:7. In embodiments, the antibody light chain is the sequence of SEQ ID NO: 7. In embodiments, the second anti-CD3 dimerizing domain includes the sequence of SEQ ID NO: 7. In embodiments, the second anti-CD3 dimerizing domain is the sequence of SEQ ID NO: 7.

In embodiments, the first anti-CD3 dimerizing domain is an antibody light chain including a CDR L1 as set forth in SEQ ID NO:32, a CDR L2 as set forth in SEQ ID NO:33 and a CDR L3 as set forth in SEQ ID NO:34. In embodiments, the first anti-CD3 dimerizing domain includes a CDR L1 as set forth in SEQ ID NO:32, a CDR L2 as set forth in SEQ ID NO:33 and a CDR L3 as set forth in SEQ ID NO:34. In embodiments, the antibody light chain includes the sequence of SEQ ID NO:38. In embodiments, the antibody light chain is the sequence of SEQ ID NO: 38. In embodiments, the first anti-CD3 dimerizing domain includes the sequence of SEQ ID NO: 38. In embodiments, the first anti-CD3 dimerizing domain is the sequence of SEQ ID NO: 38. In embodiments, the antibody light chain includes the sequence of SEQ ID NO:40. In embodiments, the antibody light chain is the sequence of SEQ ID NO:40. In embodiments, the first anti-CD3 dimerizing domain includes the sequence of SEQ ID NO:40. In embodiments, the first anti-CD3 dimerizing domain is the sequence of SEQ ID NO:40. In embodiments, the antibody light chain includes the sequence of SEQ ID NO:42. In embodiments, the antibody light chain is the sequence of SEQ ID NO:42. In embodiments, the first anti-CD3 dimerizing domain includes the sequence of SEQ ID NO:42. In embodiments, the first anti-CD3 dimerizing domain is the sequence of SEQ ID NO:42.

In embodiments, the first anti-CD3 dimerizing domain is an antibody heavy chain including a CDR H1 as set forth in SEQ ID NO:35, a CDR H2 as set forth in SEQ ID NO:36 and a CDR H3 as set forth in SEQ ID NO:37. In embodiments, the first anti-CD3 dimerizing domain includes a CDR H1 as set forth in SEQ ID NO:35, a CDR H2 as set forth in SEQ ID NO:36 and a CDR H3 as set forth in SEQ ID NO:37. In embodiments, the antibody heavy chain includes the sequence of SEQ ID NO:39. In embodiments, the antibody heavy chain is the sequence of SEQ ID NO:39. In embodiments, the first anti-CD3 dimerizing domain includes the sequence of SEQ ID NO:39. In embodiments, the first anti-CD3 dimerizing domain is the sequence of SEQ ID NO: 39. In embodiments, the antibody heavy chain includes the sequence of SEQ ID NO:41. In embodiments, the antibody heavy chain is the sequence of SEQ ID NO:41. In embodiments, the first anti-CD3 dimerizing domain includes the sequence of SEQ ID NO:41. In embodiments, the first anti-CD3 dimerizing domain is the sequence of SEQ ID NO:41. In embodiments, the antibody heavy chain includes the sequence of SEQ ID NO:43. In embodiments, the antibody heavy chain is the sequence of SEQ ID NO:43. In embodiments, the first anti-CD3 dimerizing domain includes the sequence of SEQ ID NO:43. In embodiments, the first anti-CD3 dimerizing domain is the sequence of SEQ ID NO:43.

In embodiments, the second anti-CD3 dimerizing domain is an antibody light chain including a CDR L1 as set forth in SEQ ID NO:32, a CDR L2 as set forth in SEQ ID NO:33 and a CDR L3 as set forth in SEQ ID NO:34. In embodiments, the second anti-CD3 dimerizing domain includes a CDR L1 as set forth in SEQ ID NO:32, a CDR L2 as set forth in SEQ ID NO: 33 and a CDR L3 as set forth in SEQ ID NO:34. In embodiments, the antibody light chain includes the sequence of SEQ ID NO:38. In embodiments, the antibody light chain is the sequence of SEQ ID NO:38. In embodiments, the second anti-CD3 dimerizing domain includes the sequence of SEQ ID NO:38. In embodiments, the second anti-CD3 dimerizing domain is the sequence of SEQ ID NO:38. In embodiments, the antibody light chain includes the sequence of SEQ ID NO:40. In embodiments, the antibody light chain is the sequence of SEQ ID NO:40. In embodiments, the second anti-CD3 dimerizing domain includes the sequence of SEQ ID NO:40. In embodiments, the second anti-CD3 dimerizing domain is the sequence of SEQ ID NO:40. In embodiments, the antibody light chain includes the sequence of SEQ ID NO:42. In embodiments, the antibody light chain is the sequence of SEQ ID NO:42. In embodiments, the second anti-CD3 dimerizing domain includes the sequence of SEQ ID NO:42. In embodiments, the second anti-CD3 dimerizing domain is the sequence of SEQ ID NO:42.

In embodiments, the second anti-CD3 dimerizing domain is an antibody heavy chain including a CDR H1 as set forth in SEQ ID NO:35, a CDR H2 as set forth in SEQ ID NO:36 and a CDR H3 as set forth in SEQ ID NO:37. In embodiments, the second anti-CD3 dimerizing domain includes a CDR H1 as set forth in SEQ ID NO:35, a CDR H2 as set forth in SEQ ID NO: 36 and a CDR H3 as set forth in SEQ ID NO:37. In embodiments, the antibody heavy chain includes the sequence of SEQ ID NO:39. In embodiments, the antibody heavy chain is the sequence of SEQ ID NO:39. In embodiments, the second anti-CD3 dimerizing domain includes the sequence of SEQ ID NO:39. In embodiments, the second anti-CD3 dimerizing domain is the sequence of SEQ ID NO:39. In embodiments, the antibody heavy chain includes the sequence of SEQ ID NO:41. In embodiments, the antibody heavy chain is the sequence of SEQ ID NO:41. In embodiments, the second anti-CD3 dimerizing domain includes the sequence of SEQ ID NO: 41. In embodiments, the second anti-CD3 dimerizing domain is the sequence of SEQ ID NO: 41. In embodiments, the antibody heavy chain includes the sequence of SEQ ID NO:43. In embodiments, the antibody heavy chain is the sequence of SEQ ID NO:43. In embodiments, the second anti-CD3 dimerizing domain includes the sequence of SEQ ID NO:43. In embodiments, the second anti-CD3 dimerizing domain is the sequence of SEQ ID NO:43.

In embodiments, the second anti-CD3 dimerizing domain is bound to an Fc domain through a third chemical linker. In embodiments, the anti-CD3 binding domain is an antibody including an Fc domain.

The ability of an antibody to bind a specific epitope (e.g., CD3) can be described by the equilibrium dissociation constant (K_{D}). The equilibrium dissociation constant (K_{D}) as defined herein is the ratio of the dissociation rate (K-off) and the association rate (K-on) of an antibody to CD3. It is described by the following formula: K_{D}=K-off/K-on.

In embodiments, the anti-CD3 binding domain is capable of binding a CD3 protein with an equilibrium dissociation constant (K_{D}) of less than about 650 nM. In embodiments, the anti-CD3 binding domain is capable of binding a CD3 protein with an equilibrium dissociation constant (K_{D}) of less than about 650 nM. In embodiments, the anti-CD3 binding domain is capable of binding a CD3 protein with an equilibrium dissociation constant (K_{D}) of less than about 650 nM, less than about 625 nM, less than about 600 nM, less than about 575 nM, less than about 550 nM, less than about 525 nM, less than about 500 nM, less than about 425 nM, less than about 400 nM, less than about 375 nM, less than about 350 nM, less than about 325 nM, less than about 300 nM, less than about 275 nM, less than about 250 nM, less than about 225 nM, less than about 200 nM, less than about 175 nM, less than about 150 nM, less than about 125 nM, less than about 100 nM, less than about 75 nM, less than about 50 nM, or less than about 25 nM. In embodiments, the anti-CD3 binding domain is capable of binding a CD3 protein with an equilibrium dissociation constant (K_{D}) of less than 650 nM, less than 625 nM, less than 600 nM, less than 575 nM, less than 550 nM, less than 525 nM, less than 500 nM, less than 425 nM, less than 400 nM, less than 375 nM, less than 350 nM, less than 325 nM, less than 300 nM, less than 275 nM, less than 250 nM, less than 225 nM, less than 200 nM, less than 175 nM, less than 150 nM, less than 125 nM, less than 100 nM, less than 75 nM, less than 50 nM, or less than 25 nM.

In embodiments, the anti-CD3 binding domain is capable of binding a CD3 protein with an equilibrium dissociation constant (K_{D}) of about 530 nM. In embodiments, the anti-CD3 binding domain is capable of binding a CD3 protein with an equilibrium dissociation constant (K_{D}) of 530 nM. In embodiments, the anti-CD3 binding domain is capable of binding a CD3 protein with an equilibrium dissociation constant (K_{D}) of about 522 nM. In embodiments, the anti-CD3 binding domain is capable of binding a CD3 protein with an equilibrium dissociation constant (K_{D}) of 522 nM.

In embodiments, the anti-CD3 binding domain is capable of binding a CD3 protein with an equilibrium dissociation constant (K_{D}) of about 400 nM to about 650 nM. In embodiments, the anti-CD3 binding domain is capable of binding a CD3 protein with an equilibrium dissociation constant (K_{D}) of about 425 nM to about 650 nM. In embodiments, the anti-CD3 binding domain is capable of binding a CD3 protein with an equilibrium dissociation constant (K_{D}) of about 450 nM to about 650 nM. In embodiments, the anti-CD3 binding domain is capable of binding a CD3 protein with an equilibrium dissociation constant (K_{D}) of about 475 nM to about 650 nM. In embodiments, the anti-CD3 binding domain is capable of binding a CD3 protein with an equilibrium dissociation constant (K_{D}) of about 500 nM to about 650 nM. In embodiments, the anti-CD3 binding domain is capable of binding a CD3 protein with an equilibrium dissociation constant (K_{D}) of about 525 nM to about 650 nM. In embodiments, the anti-CD3 binding domain is capable of binding a CD3 protein with an equilibrium dissociation constant (K_{D}) of about 550 nM to about 650 nM. In embodiments, the anti-CD3 binding domain is capable of binding a CD3 protein with an equilibrium dissociation constant (K_{D}) of about 575 nM to about 650 nM. In embodiments, the anti-CD3 binding domain is capable of binding a CD3 protein with an equilibrium dissociation constant (K_{D}) of about 600 nM to about 650 nM. In embodiments, the anti-CD3 binding domain is capable of binding a CD3 protein with an equilibrium dissociation constant (K_{D}) of about 625 nM to about 650 nM.

In embodiments, the anti-CD3 binding domain is capable of binding a CD3 protein with an equilibrium dissociation constant (K_{D}) of about 400 nM to about 625 nM. In embodiments, the anti-CD3 binding domain is capable of binding a CD3 protein with an equilibrium dissociation constant (K_{D}) of about 400 nM to about 600 nM. In embodiments, the anti-CD3 binding domain is capable of binding a CD3 protein with an equilibrium dissociation constant (K_{D}) of about 400 nM to about 575 nM. In embodiments, the anti-CD3 binding domain is capable of binding a CD3 protein with an equilibrium dissociation constant (K_{D}) of about 400 nM to about 550 nM. In embodiments, the anti-CD3 binding domain is capable of binding a CD3 protein with an equilibrium dissociation constant (K_{D}) of about 400 nM to about 525 nM. In embodiments, the anti-CD3 binding domain is capable of binding a CD3 protein with an equilibrium dissociation constant (K_{D}) of about 400 nM to about 500 nM. In embodiments, the anti-CD3 binding domain is capable of binding a CD3 protein with an equilibrium dissociation constant (K_{D}) of about 400 nM to about 475 nM. In embodiments, the anti-CD3 binding domain is capable of binding a CD3 protein with an equilibrium dissociation constant (K_{D}) of about 400 nM to about 450 nM. In embodiments, the anti-CD3 binding domain is capable of binding a CD3 protein with an equilibrium dissociation constant (K_{D}) of about 400 nM to about 425 nM. In embodiments, the anti-CD3 binding domain is capable of binding a CD3 protein with an equilibrium dissociation constant (K_{D}) of about 400 nM, about 425 nM, about 450 nM, about 475 nM, about 500 nM, about 525 nM, about 550 nM, about 575 nM, about 600 nM, about 625 nM, or about 600 nM. In embodiments, the anti-CD3 binding domain is capable of binding a CD3 protein with an equilibrium dissociation constant (K_{D}) of 400 nM, 425 nM, 450 nM, 475 nM, 500 nM, 525 nM, 550 nM, 575 nM, 600 nM, 625 nM or 650 nM.

In embodiments, the first chemical linker and the second chemical linker are independently a covalent linker or a non-covalent linker. In embodiments, the first chemical linker and the second chemical linker are independently a covalent linker. In embodiments, the first chemical linker and the second chemical linker are independently a non-covalent linker. In embodiments, the first chemical linker and the second chemical linker are independently a cleavable peptide linker. In embodiments, said first chemical linker and said second chemical linker are independently an enzymatically cleavable linker. In embodiments, the first chemical linker and the second chemical linker are independently a protease cleavable linker.

In embodiments, the first chemical linker and the second chemical linker are independently a cleavable peptide linker, including a protease cleavage site. A “cleavage site” as used herein, refers to a recognizable site for cleavage of a portion of a linker described herein. Thus, a cleavage site may be found in the sequence of a cleavable peptide linker as described herein, including embodiments thereof. In embodiments, the cleavage site is an amino acid sequence that is recognized and cleaved by a cleaving agent (e.g., a peptidyl sequence). Exemplary cleaving agents include proteins, enzymes, DNAzymes, RNAzymes, metals, acids, and bases. In embodiments, the protease cleavage site is a tumor-associated protease cleavage site. A “tumor-associated protease cleavage site” as provided herein is an amino acid sequence recognized by a protease, whose expression is specific for a tumor cell or tumor cell environment thereof. In embodiments, the protease cleavage site is a matrix metalloprotease (MMP) cleavage site, a disintegrin and metalloprotease domain-containing (ADAM) metalloprotease cleavage site, a prostate specific antigen (PSA) protease cleavage site, a urokinase-type plasminogen activator (uPA) protease cleavage site, a membrane type serine protease 1 (MT-SP1) protease cleavage site or a legumain protease cleavage site. In embodiments, the matrix metalloprotease (MMP) cleavage site is a MMP 9 cleavage site, a MMP 13 cleavage site or a MMP 2 cleavage site. In embodiments, the disintegrin and metalloprotease domain-containing (ADAM) metalloprotease cleavage site is a ADAM 9 metalloprotease cleavage site, a ADAM 10 metalloprotease cleavage site or a ADAM 17 metalloprotease cleavage site.

Further exemplary cleavage sites include the cleavage site of ABHD12, ADAM12, ABHD12B, ABHD13, ABHD17A, ADAM19, ADAM20, ADAM21, ADAM28, ADAM30, ADAM33, ADAM8, ABHD17A, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ABHD17B, ADAMTS5, ADAMTS6, ADAMTS7, ADAMTS8, ADAMTS9, ADAMTSL1, ADAMTSL2, ADAMTSL3, ABHD17C, ADAMTSL5, ASTL, BMP1, CELA1, CELA2A, CELA2B, CELA3A, CELA3B, ADAM10, ADAM15, ADAM17, ADAM9, ADAMTS4, CTSE, CTSF, ADAMTSL4, CMA1, CTRB1, CTRC, CTSO, CTRI, CTSA, CTSW, CTSB, CTSC, CTSD, ESP1, CTSG, CTSH, GZMA, GZMB, GZMH, CTSK, GZMM, CTSL, CTSS, CTSV, CTSZ, HTRA4, KLK10, KLK11, KLK13, KLK14, KLK2, KLK4, DPP4, KLK6, KLK7, KLKB1, ECE1, ECE2, ECEL1, MASP2, MEP1A, MEP1B, ELANE, FAP, GZMA, MMP11, GZMK, HGFAC, HPN, HTRA1, MMP11, MMP16, MMP17, MMP19, HTRA2, MMP20, MMP21, HTRA3, HTRA4, KEL, MMP23B, MMP24, MMP25, MMP26, MMP27, MMP28, KLK5, MMP3, MMP7, MMP8, MMP9, LGMN, LNPEP, MASP1, PAPPA, PAPPA2, PCSK1, NAPSA, PCSK5, PCSK6, MME, MMP1, MMP10, PLAT, PLAU, PLG, PRSS1, PRSS12, PRSS2, PRSS21, PRSS3, PRSS33, PRSS4, PRSS55, PRSS57, MMP12, PRSS8, PRSS9, PRTN3, MMP13, MMP14, ST14, TMPRSS10, TMPRSS11A, TMPRSS11D, TMPRSS11E, TMPRSS11F, TMPRSS12, TMPRSS13, MMP15, TMPRSS15, MMP2, TMPRSS2, TMPRSS3, TMPRSS4, TMPRSS5, TMPRSS6, TMPRSS7, TMPRSS9, NRDC, OVCH1, PAMR1, PCSK3, PHEX, TINAG, TPSAB1, TPSD1, or TPSG1.

The chemical linkers provided herein, including embodiments thereof, may have different lengths (e.g., include varying numbers of amino acid residues). Thus, in embodiments, the first chemical linker and the second chemical linker independently have a length of about 0 to about 15 amino acid residues. In embodiments, the first chemical linker and the second chemical linker independently have a length of about 2 to about 15 amino acid residues. In embodiments, the first chemical linker and the second chemical linker independently have a length of about 3 to about 15 amino acid residues. In embodiments, the first chemical linker and the second chemical linker independently have a length of about 4 to about 15 amino acid residues. In embodiments, the first chemical linker and the second chemical linker independently have a length of about 5 to about 15 amino acid residues. In embodiments, the first chemical linker and the second chemical linker independently have a length of about 6 to about 15 amino acid residues. In embodiments, the first chemical linker and the second chemical linker independently have a length of about 7 to about 15 amino acid residues. In embodiments, the first chemical linker and the second chemical linker independently have a length of about 8 to about 15 amino acid residues. In embodiments, the first chemical linker and the second chemical linker independently have a length of about 9 to about 15 amino acid residues. In embodiments, the first chemical linker and the second chemical linker independently have a length of about 10 to about 15 amino acid residues. In embodiments, the first chemical linker and the second chemical linker independently have a length of about 11 to about 15 amino acid residues. In embodiments, the first chemical linker and the second chemical linker independently have a length of about 12 to about 15 amino acid residues. In embodiments, the first chemical linker and the second chemical linker independently have a length of about 14 to about 15 amino acid residues.

In embodiments, the first chemical linker and the second chemical linker independently have a length of about 0 to about 14 amino acid residues. In embodiments, the first chemical linker and the second chemical linker independently have a length of about 0 to about 13 amino acid residues. In embodiments, the first chemical linker and the second chemical linker independently have a length of about 0 to about 12 amino acid residues. In embodiments, the first chemical linker and the second chemical linker independently have a length of about 0 to about 11 amino acid residues. In embodiments, the first chemical linker and the second chemical linker independently have a length of about 0 to about 10 amino acid residues. In embodiments, the first chemical linker and the second chemical linker independently have a length of about 0 to about 9 amino acid residues. In embodiments, the first chemical linker and the second chemical linker independently have a length of about 0 to about 8 amino acid residues. In embodiments, the first chemical linker and the second chemical linker independently have a length of about 0 to about 7 amino acid residues. In embodiments, the first chemical linker and the second chemical linker independently have a length of about 0 to about 6 amino acid residues. In embodiments, the first chemical linker and the second chemical linker independently have a length of about 0 to about 5 amino acid residues. In embodiments, the first chemical linker and the second chemical linker independently have a length of about 0 to about 4 amino acid residues. In embodiments, the first chemical linker and the second chemical linker independently have a length of about 0 to about 3 amino acid residues. In embodiments, the first chemical linker and the second chemical linker independently have a length of about 0 to about 2 amino acid residues. In embodiments, the first chemical linker and the second chemical linker independently have a length of about 0 to about 1 amino acid residues. In embodiments, the first chemical linker and the second chemical linker independently have a length of about 0, about 1, about 2, about 3, about 4, about 5, about 6, about 7, about 8, about 9, about 10, about 11, about 12, about 13, about 14, or about 15 residues. In embodiments, the first chemical linker and the second chemical linker independently have a length of 0, 1, 2, 3, 4, 5, 6, 7, 8, 9, 10, 11, 12, 13, 14, or 15 residues.

In embodiments, the peptide includes from the N-terminus to the C-terminus an anti-CD3 variable heavy chain domain, an anti-CD3 constant heavy chain domain, a first chemical linker, a CD38 single domain antibody, a second chemical linker, an anti-CD3 variable light chain domain, and an anti-CD3 constant light chain domain. In embodiments, the peptide includes from the N-terminus to the C-terminus an anti-CD3 variable light chain domain, an anti-CD3 constant light chain domain, a first chemical linker, a CD38 single domain antibody, a second chemical linker, an anti-CD3 variable heavy chain domain, and an anti-CD3 constant heavy chain domain.

In embodiments, the first chemical linker has a length of about 6 to about 10 amino acids. In embodiments, the first chemical linker has a length of about 7 to about 10 amino acids. In embodiments, the first chemical linker has a length of about 8 to about 10 amino acids. In embodiments, the first chemical linker has a length of 6 to 10 amino acids. In embodiments, the first chemical linker has a length from 7 to 10 amino acids. In embodiments, the first chemical linker has a length of 8 to 10 amino acids. In embodiments, the first chemical linker has a length of about 8 amino acids. In embodiments, the first chemical linker has a length of 8 amino acids. In embodiments, first chemical linker includes the sequence of SEQ ID NO:9. In embodiments, first chemical linker is the sequence of SEQ ID NO:9.

In embodiments, the second chemical linker has a length of about 10 to about 16 amino acids. In embodiments, the second chemical linker has a length of about 11 to about 16 amino acids. In embodiments, the second chemical linker has a length of about 12 to about 16 amino acids. In embodiments, the second chemical linker has a length of about 13 to about 16 amino acids. In embodiments, the second chemical linker has a length of about 14 to about 16 amino acids. In embodiments, the second chemical linker has a length of 10 to 16 amino acids. In embodiments, the second chemical linker has a length of 11 to 16 amino acids. In embodiments, the second chemical linker has a length of 12 to 16 amino acids. In embodiments, the second chemical linker has a length of 13 to 16 amino acids. In embodiments, the second chemical linker has a length of 14 to 16 amino acids.

In embodiments, the second chemical linker has a length of about 12 amino acids. In embodiments, the second chemical linker has a length of 12 amino acids. In embodiments, the second chemical linker has a length of about 14 amino acids. In embodiments, the second chemical linker has a length of 14 amino acids. In embodiments, the second chemical linker includes the sequence of SEQ ID NO:10 SEQ, SEQ ID NO: 11, SEQ ID NO:30 or SEQ ID NO: 31. In embodiments, the second chemical linker is the sequence of SEQ ID NO:10, SEQ ID NO: 11, SEQ ID NO:30 or SEQ ID NO:31. In embodiments, the second chemical linker includes the sequence of SEQ ID NO:10. In embodiments, the second chemical linker is the sequence of SEQ ID NO:10. In embodiments, the second chemical linker includes the sequence of SEQ ID NO: 11. In embodiments, the second chemical linker is the sequence of SEQ ID NO:11. In embodiments, the second chemical linker includes the sequence of SEQ ID NO:30. In embodiments, the second chemical linker is the sequence of SEQ ID NO:30. In embodiments, the second chemical linker includes the sequence of SEQ ID NO:31. In embodiments, the second chemical linker is the sequence of SEQ ID NO:31.

In embodiments, the CD38 ligand binding domain is a single domain antibody. In embodiments, the CD38 ligand binding domain is a nanobody.

In embodiments, the CD38 ligand binding domain includes a CDR L1 as set forth in SEQ ID NO:12, a CDR L2 as set forth in SEQ ID NO: 13 and a CDR L3 as set forth in SEQ ID NO: 14; a CDR L1 as set forth in SEQ ID NO:22, a CDR L2 as set forth in SEQ ID NO:23 and a CDR L3 as set forth in SEQ ID NO:24; or a CDR L1 as set forth in SEQ ID NO:26, a CDR L2 as set forth in SEQ ID NO:27 and a CDR L3 as set forth in SEQ ID NO:28. In embodiments, the CD38 ligand binding domain includes a CDR L1 as set forth in SEQ ID NO:12, a CDR L2 as set forth in SEQ ID NO:13 and a CDR L3 as set forth in SEQ ID NO:14. In embodiments, the CD38 ligand binding domain includes a CDR L1 as set forth in SEQ ID NO:22, a CDR L2 as set forth in SEQ ID NO:23 and a CDR L3 as set forth in SEQ ID NO:24. In embodiments, the CD38 ligand binding domain includes a CDR L1 as set forth in SEQ ID NO:26, a CDR L2 as set forth in SEQ ID NO:27 and a CDR L3 as set forth in SEQ ID NO:28.

In embodiments, the CD38 ligand binding domain includes the sequence of SEQ ID NO: 15, SEQ ID NO:25 or SEQ ID NO:29. In embodiments, the CD38 ligand binding domain is the sequence of SEQ ID NO:15, SEQ ID NO:25 or SEQ ID NO:29. In embodiments, the CD38 ligand binding domain includes the sequence of SEQ ID NO:15. In embodiments, the CD38 ligand binding domain includes the sequence of SEQ ID NO:25. In embodiments, the CD38 ligand binding domain includes the sequence of SEQ ID NO:29. In embodiments, the CD38 ligand binding domain is the sequence of SEQ ID NO:15. In embodiments, the CD38 ligand binding domain is the sequence of SEQ ID NO:25. In embodiments, the CD38 ligand binding domain is the sequence of SEQ ID NO:29.

In embodiments, the peptide includes the sequence of SEQ ID NO:16, SEQ ID NO:17, SEQ ID NO:18, SEQ ID NO:19, SEQ ID NO:20 or SEQ ID NO:21. In embodiments, the peptide includes the sequence of SEQ ID NO:16. In embodiments, the peptide includes the sequence of SEQ ID NO: 17. In embodiments, the peptide includes the sequence of SEQ ID SEQ ID NO: 18. In embodiments, the peptide includes the sequence of SEQ ID NO:19. In embodiments, the peptide includes the sequence of SEQ ID NO:20. In embodiments, the peptide includes the sequence of SEQ ID NO:21. In embodiments, the peptide is the sequence of SEQ ID NO: 16, SEQ ID NO:17, SEQ ID NO: 18, SEQ ID NO:19, SEQ ID NO:20 or SEQ ID NO:21. In embodiments, the peptide is the sequence of SEQ ID NO:16. In embodiments, the peptide is the sequence of SEQ ID NO:17. In embodiments, the peptide is the sequence of SEQ ID SEQ ID NO: 18. In embodiments, the peptide is the sequence of SEQ ID NO:19. In embodiments, the peptide is the sequence of SEQ ID NO:20. In embodiments, the peptide is the sequence of SEQ ID NO: 21.

In one embodiment, the peptide includes from the N-Terminus to the C-terminus a first anti-CD3 dimerizing domain including the sequence of SEQ ID NO:8, SEQ ID NO:39, SEQ ID NO: 41 or SEQ ID NO:43; a first chemical linker including the sequence of SEQ ID NO:9; a second anti-CD3 dimerizing domain including the sequence of SEQ ID NO:7, SEQ ID NO:38, SEQ ID NO:40 or SEQ ID NO:42; a CD38 binding domain including the sequence of SEQ ID NO: 15, SEQ ID NO:25 or SEQ ID NO:29 and a second chemical linker including the sequence of SEQ ID NO:10, SEQ ID NO:11, SEQ ID NO:30 or SEQ ID NO:31.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:15 and a second chemical linker including the sequence of SEQ ID NO:10.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:25 and a second chemical linker including the sequence of SEQ ID NO:10.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:29 and a second chemical linker including the sequence of SEQ ID NO:10.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:15 and a second chemical linker including the sequence of SEQ ID NO:11.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:25 and a second chemical linker including the sequence of SEQ ID NO:11.

In one embodiment, the peptide includes from the N-Terminus to the C-terminus a first anti-CD3 dimerizing domain including the sequence of SEQ ID NO:8; a first chemical linker including the sequence of SEQ ID NO:9; a second anti-CD3 dimerizing domain including the sequence of SEQ ID NO:7; a CD38 binding domain including the sequence of SEQ ID NO:29 and a second chemical linker including the sequence of SEQ ID NO:11.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:15 and a second chemical linker including the sequence of SEQ ID NO:10.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:25 and a second chemical linker including the sequence of SEQ ID NO:10.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:29 and a second chemical linker including the sequence of SEQ ID NO:10.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:15 and a second chemical linker including the sequence of SEQ ID NO:11.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:25 and a second chemical linker including the sequence of SEQ ID NO:11.

In one embodiment, the peptide includes from the N-Terminus to the C-terminus a first anti-CD3 dimerizing domain including the sequence of SEQ ID NO:39; a first chemical linker including the sequence of SEQ ID NO:9; a second anti-CD3 dimerizing domain including the sequence of SEQ ID NO:38; a CD38 binding domain including the sequence of SEQ ID NO:29 and a second chemical linker including the sequence of SEQ ID NO:11.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:15 and a second chemical linker including the sequence of SEQ ID NO:10.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:25 and a second chemical linker including the sequence of SEQ ID NO:10.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:29 and a second chemical linker including the sequence of SEQ ID NO:10.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:15 and a second chemical linker including the sequence of SEQ ID NO:11.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:25 and a second chemical linker including the sequence of SEQ ID NO:11.

In one embodiment, the peptide includes from the N-Terminus to the C-terminus a first anti-CD3 dimerizing domain including the sequence of SEQ ID NO:41; a first chemical linker including the sequence of SEQ ID NO:9; a second anti-CD3 dimerizing domain including the sequence of SEQ ID NO:40; a CD38 binding domain including the sequence of SEQ ID NO:29 and a second chemical linker including the sequence of SEQ ID NO:11.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:15 and a second chemical linker including the sequence of SEQ ID NO:10.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:25 and a second chemical linker including the sequence of SEQ ID NO:10.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:29 and a second chemical linker including the sequence of SEQ ID NO:10.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:15 and a second chemical linker including the sequence of SEQ ID NO:11.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:25 and a second chemical linker including the sequence of SEQ ID NO:11.

In one embodiment, the peptide includes from the N-Terminus to the C-terminus a first anti-CD3 dimerizing domain including the sequence of SEQ ID NO:43; a first chemical linker including the sequence of SEQ ID NO:9; a second anti-CD3 dimerizing domain including the sequence of SEQ ID NO:42; a CD38 binding domain including the sequence of SEQ ID NO:29 and a second chemical linker including the sequence of SEQ ID NO:11.

The ability of a binding domain to bind a specific protein (e.g., CD38, CD3) can be described by the equilibrium dissociation constant (K_{D}). The equilibrium dissociation constant (K_{D}) as defined herein is the ratio of the dissociation rate (K-off) and the association rate (K-on) of an CD38 ligand binding domain to CD38. It is described by the following formula: K_{D}=K-off/K-on. In embodiments, the CD38 ligand binding domain is capable of binding a CD38 protein with an equilibrium dissociation constant (K_{D}) of less than about 1.5 nM. In embodiments, the CD38 ligand binding domain is capable of binding a CD38 protein with an equilibrium dissociation constant (K_{D}) of less than about 1.5 nM, less than about 1.25 nM, less than about 1 nM, less than about 0.75 nM, less than about 0.5 nM, or less than about 0.25 nM. In embodiments, the CD38 ligand binding domain is capable of binding a CD38 protein with an equilibrium dissociation constant (K_{D}) of less than 1.5 nM, less than 1.25 nM, less than 1 nM, less than 0.75 nM, less than 0.5 nM, or less than 0.25 nM. In embodiments, the CD38 ligand binding domain is capable of binding a CD38 protein with an equilibrium dissociation constant (K_{D}) of less than 1.5 nM. In embodiments, the CD38 ligand binding domain is capable of binding a CD38 protein with an equilibrium dissociation constant (K_{D}) of less than 1.5 nM, less than 1.25 nM, less than 1 nM, less than 0.75 nM, less than 0.5 nM, or less than 0.25 nM.

In embodiments, the CD38 ligand binding domain is capable of binding a CD38 protein with an equilibrium dissociation constant (K_{D}) of less than 1.5 nM. In embodiments, the CD38 ligand binding domain is capable of binding a CD38 protein with an equilibrium dissociation constant (K_{D}) of about 0.946 nM. In embodiments, the CD38 ligand binding domain is capable of binding a CD38 protein with an equilibrium dissociation constant (K_{D}) of 0.946 nM.

In embodiments, the CD38 ligand binding domain is capable of binding a CD38 protein with an equilibrium dissociation constant (K_{D}) of about 0.772 nM. In embodiments, the CD38 ligand binding domain is capable of binding a CD38 protein with an equilibrium dissociation constant (K_{D}) of 0.772 nM.

In embodiments, the CD38 ligand binding domain is capable of binding a CD38 protein with an equilibrium dissociation constant (K_{D}) of about 0.478 nM. In embodiments, the CD38 ligand binding domain is capable of binding a CD38 protein with an equilibrium dissociation constant (K_{D}) of 0.478 nM.

In embodiments, the CD38 ligand binding domain is capable of binding a CD38 protein with an equilibrium dissociation constant (K_{D}) of about 0.962 nM. In embodiments, the CD38 ligand binding domain is capable of binding a CD38 protein with an equilibrium dissociation constant (K_{D}) of 0.962 nM.

In embodiments, the anti-CD38 binding domain is capable of binding a CD38 protein with an equilibrium dissociation constant (K_{D}) of about 0.4735 nM. In embodiments, the anti-CD38 binding domain is capable of binding a CD38 protein with an equilibrium dissociation constant (K_{D}) of 0.4735 nM.

In embodiments, the CD38 ligand binding domain is capable of binding a CD38 protein with an equilibrium dissociation constant (K_{D}) of about 0.547 nM. In embodiments, CD38 ligand binding domain is capable of binding a CD38 protein with an equilibrium dissociation constant (K_{D}) of 0.547 nM.

In embodiments, the CD38 ligand binding domain is capable of binding a CD38 protein with an equilibrium dissociation constant (K_{D}) of about 0.5 nM to about 1.5 nM. In embodiments, the CD38 ligand binding domain is capable of binding a CD38 protein with an equilibrium dissociation constant (K_{D}) of about 0.6 nM to about 1.5 nM. In embodiments, the CD38 ligand binding domain is capable of binding a CD38 protein with an equilibrium dissociation constant (K_{D}) of about 0.7 nM to about 1.5 nM. In embodiments, the CD38 ligand binding domain is capable of binding a CD38 protein with an equilibrium dissociation constant (K_{D}) of about 0.8 nM to about 1.5 nM. In embodiments, the CD38 ligand binding domain is capable of binding a CD38 protein with an equilibrium dissociation constant (K_{D}) of about 0.9 nM to about 1.5 nM. In embodiments, the CD38 ligand binding domain is capable of binding a CD38 protein with an equilibrium dissociation constant (K_{D}) of about 1 nM to about 1.5 nM. In embodiments, the CD38 ligand binding domain is capable of binding a CD38 protein with an equilibrium dissociation constant (K_{D}) of about 1.1 nM to about 1.5 nM. In embodiments, the CD38 ligand binding domain is capable of binding a CD38 protein with an equilibrium dissociation constant (K_{D}) of about 1.2 nM to about 1.5 nM. In embodiments, the CD38 ligand binding domain is capable of binding a CD38 protein with an equilibrium dissociation constant (K_{D}) of about 1.3 nM to about 1.5 nM. In embodiments, the CD38 ligand binding domain is capable of binding a CD38 protein with an equilibrium dissociation constant (K_{D}) of about 1.4 nM to about 1.5 nM.

In embodiments, the CD38 ligand binding domain is capable of binding a CD38 protein with an equilibrium dissociation constant (K_{D}) of about 0.5 nM to about 1.4 nM. In embodiments, the CD38 ligand binding domain is capable of binding a CD38 protein with an equilibrium dissociation constant (K_{D}) of about 0.5 nM to about 1.3 nM. In embodiments, the CD38 ligand binding domain is capable of binding a CD38 protein with an equilibrium dissociation constant (K_{D}) of about 0.5 nM to about 1.2 nM. In embodiments, the CD38 ligand binding domain is capable of binding a CD38 protein with an equilibrium dissociation constant (K_{D}) of about 0.5 nM to about 1.1 nM. In embodiments, the CD38 ligand binding domain is capable of binding a CD38 protein with an equilibrium dissociation constant (K_{D}) of about 0.5 nM to about 1 nM. In embodiments, the CD38 ligand binding domain is capable of binding a CD38 protein with an equilibrium dissociation constant (K_{D}) of about 0.5 nM to about 0.9 nM. In embodiments, the CD38 ligand binding domain is capable of binding a CD38 protein with an equilibrium dissociation constant (K_{D}) of about 0.5 nM to about 0.8 nM. In embodiments, the CD38 ligand binding domain is capable of binding a CD38 protein with an equilibrium dissociation constant (K_{D}) of about 0.5 nM to about 0.7 nM. In embodiments, the CD38 ligand binding domain is capable of binding a CD38 protein with an equilibrium dissociation constant (K_{D}) of about 0.5 nM to about 0.6 nM. In embodiments, the CD38 ligand binding domain is capable of binding a CD38 protein with an equilibrium dissociation constant (K_{D}) of about 0.5 nM, about 0.6 nM, about 0.7 nM, about 0.8 nM, about 0.9 nM, about 1 nM, about 1.1 nM, about 1.2 nM, about 1.3 nM, about 1.4 nM, or about 1.5 nM. In embodiments, the CD38 ligand binding domain is capable of binding a CD38 protein with an equilibrium dissociation constant (K_{D}) of 0.5 nM, 0.6 nM, 0.7 nM, 0.8 nM, 0.9 nM, 1 nM, 1.1 nM, 1.2 nM, 1.3 nM, 1.4 nM, or 1.5 nM.

In embodiments, the CD38 ligand binding domain is capable of binding a CD38 protein with an equilibrium dissociation constant (K_{D}) of 0.5 nM to 1.5 nM. In embodiments, the CD38 ligand binding domain is capable of binding a CD38 protein with an equilibrium dissociation constant (K_{D}) of 0.6 nM to 1.5 nM. In embodiments, the CD38 ligand binding domain is capable of binding a CD38 protein with an equilibrium dissociation constant (K_{D}) of 0.7 nM to 1.5 nM. In embodiments, the CD38 ligand binding domain is capable of binding a CD38 protein with an equilibrium dissociation constant (K_{D}) of 0.8 nM to 1.5 nM. In embodiments, the CD38 ligand binding domain is capable of binding a CD38 protein with an equilibrium dissociation constant (K_{D}) of 0.9 nM to 1.5 nM. In embodiments, the CD38 ligand binding domain is capable of binding a CD38 protein with an equilibrium dissociation constant (K_{D}) of 1 nM to 1.5 nM. In embodiments, the CD38 ligand binding domain is capable of binding a CD38 protein with an equilibrium dissociation constant (K_{D}) of 1.1 nM to 1.5 nM. In embodiments, the CD38 ligand binding domain is capable of binding a CD38 protein with an equilibrium dissociation constant (K_{D}) of 1.2 nM to 1.5 nM. In embodiments, the CD38 ligand binding domain is capable of binding a CD38 protein with an equilibrium dissociation constant (K_{D}) of 1.3 nM to 1.5 nM. In embodiments, the CD38 ligand binding domain is capable of binding a CD38 protein with an equilibrium dissociation constant (K_{D}) of 1.4 nM to 1.5 nM.

In embodiments, the CD38 ligand binding domain is capable of binding a CD38 protein with an equilibrium dissociation constant (K_{D}) of 0.5 nM to 1.4 nM. In embodiments, the CD38 ligand binding domain is capable of binding a CD38 protein with an equilibrium dissociation constant (K_{D}) of 0.5 nM to 1.3 nM. In embodiments, the CD38 ligand binding domain is capable of binding a CD38 protein with an equilibrium dissociation constant (K_{D}) of 0.5 nM to 1.2 nM. In embodiments, the CD38 ligand binding domain is capable of binding a CD38 protein with an equilibrium dissociation constant (K_{D}) of 0.5 nM to 1.1 nM. In embodiments, the CD38 ligand binding domain is capable of binding a CD38 protein with an equilibrium dissociation constant (K_{D}) of 0.5 nM to 1 nM. In embodiments, the CD38 ligand binding domain is capable of binding a CD38 protein with an equilibrium dissociation constant (K_{D}) of 0.5 nM to 0.9 nM. In embodiments, the CD38 ligand binding domain is capable of binding a CD38 protein with an equilibrium dissociation constant (K_{D}) of 0.5 nM to 0.8 nM. In embodiments, the CD38 ligand binding domain is capable of binding a CD38 protein with an equilibrium dissociation constant (K_{D}) of 0.5 nM to 0.7 nM. In embodiments, the CD38 ligand binding domain is capable of binding a CD38 protein with an equilibrium dissociation constant (K_{D}) of 0.5 nM to 0.6 nM. In embodiments, the CD38 ligand binding domain is capable of binding a CD38 protein with an equilibrium dissociation constant (K_{D}) of 0.5 nM, 0.6 nM, 0.7 nM, 0.8 nM, 0.9 nM, 1 nM, 1.1 nM, 1.2 nM, 1.3 nM, 1.4 nM, or 1.5 nM.

In embodiments, the CD38 ligand binding domain is capable of binding a CD38 protein with an equilibrium dissociation constant (K_{D}) of about 0.1 nM to about 0.8 nM. In embodiments, the CD38 ligand binding domain is capable of binding a CD38 protein with an equilibrium dissociation constant (K_{D}) of about 0.2 nM to about 0.8 nM. In embodiments, the CD38 ligand binding domain is capable of binding a CD38 protein with an equilibrium dissociation constant (K_{D}) of about 0.3 nM to about 0.8 nM. In embodiments, the CD38 ligand binding domain is capable of binding a CD38 protein with an equilibrium dissociation constant (K_{D}) of about 0.4 nM to about 0.8 nM. In embodiments, the CD38 ligand binding domain is capable of binding a CD38 protein with an equilibrium dissociation constant (K_{D}) of about 0.5 nM to about 0.8 nM. In embodiments, the CD38 ligand binding domain is capable of binding a CD38 protein with an equilibrium dissociation constant (K_{D}) of about 0.6 nM to about 0.8 nM. In embodiments, the CD38 ligand binding domain is capable of binding a CD38 protein with an equilibrium dissociation constant (K_{D}) of about 0.7 nM to about 0.8 nM.

In embodiments, the CD38 ligand binding domain is capable of binding a CD38 protein with an equilibrium dissociation constant (K_{D}) of about 0.1 nM to about 0.7 nM. In embodiments, the CD38 ligand binding domain is capable of binding a CD38 protein with an equilibrium dissociation constant (K_{D}) of about 0.1 nM to about 0.6 nM. In embodiments, the CD38 ligand binding domain is capable of binding a CD38 protein with an equilibrium dissociation constant (K_{D}) of about 0.1 nM to about 0.5 nM. In embodiments, the CD38 ligand binding domain is capable of binding a CD38 protein with an equilibrium dissociation constant (K_{D}) of about 0.1 nM to about 0.4 nM. In embodiments, the CD38 ligand binding domain is capable of binding a CD38 protein with an equilibrium dissociation constant (K_{D}) of about 0.1 nM to about 0.3 nM. In embodiments, the CD38 ligand binding domain is capable of binding a CD38 protein with an equilibrium dissociation constant (K_{D}) of about 0.1 nM to about 0.2 nM. In embodiments, the CD38 ligand binding domain is capable of binding a CD38 protein with an equilibrium dissociation constant (K_{D}) of about 0.1 nM, about 0.2 nM, about 0.3 nM, about 0.4 nM, about 0.5 nM, about 0.6 nM, about 0.7 nM, or about 0.8 nM. In embodiments, the CD38 ligand binding domain is capable of binding a CD38 protein with an equilibrium dissociation constant (K_{D}) of 0.1 nM, 0.2 nM, 0.3 nM, 0.4 nM, 0.5 nM, 0.6 nM, 0.7 nM, or 0.8 nM.

In embodiments, the CD38 ligand binding domain is capable of binding a CD38 protein with an equilibrium dissociation constant (K_{D}) of 0.1 nM to 0.8 nM. In embodiments, the CD38 ligand binding domain is capable of binding a CD38 protein with an equilibrium dissociation constant (K_{D}) of 0.2 nM to 0.8 nM. In embodiments, the CD38 ligand binding domain is capable of binding a CD38 protein with an equilibrium dissociation constant (K_{D}) of 0.3 nM to 0.8 nM. In embodiments, the CD38 ligand binding domain is capable of binding a CD38 protein with an equilibrium dissociation constant (K_{D}) of 0.4 nM to 0.8 nM. In embodiments, the CD38 ligand binding domain is capable of binding a CD38 protein with an equilibrium dissociation constant (K_{D}) of 0.5 nM to 0.8 nM. In embodiments, the CD38 ligand binding domain is capable of binding a CD38 protein with an equilibrium dissociation constant (K_{D}) of 0.6 nM to 0.8 nM. In embodiments, the CD38 ligand binding domain is capable of binding a CD38 protein with an equilibrium dissociation constant (K_{D}) of 0.7 nM to 0.8 nM.

In embodiments, the CD38 ligand binding domain is capable of binding a CD38 protein with an equilibrium dissociation constant (K_{D}) of 0.1 nM to 0.7 nM. In embodiments, the CD38 ligand binding domain is capable of binding a CD38 protein with an equilibrium dissociation constant (K_{D}) of 0.1 nM to 0.6 nM. In embodiments, the CD38 ligand binding domain is capable of binding a CD38 protein with an equilibrium dissociation constant (K_{D}) of 0.1 nM to 0.5 nM. In embodiments, the CD38 ligand binding domain is capable of binding a CD38 protein with an equilibrium dissociation constant (K_{D}) of 0.1 nM to 0.4 nM. In embodiments, the CD38 ligand binding domain is capable of binding a CD38 protein with an equilibrium dissociation constant (K_{D}) of 0.1 nM to 0.3 nM. In embodiments, the CD38 ligand binding domain is capable of binding a CD38 protein with an equilibrium dissociation constant (K_{D}) of 0.1 nM to 0.2 nM. In embodiments, the CD38 ligand binding domain is capable of binding a CD38 protein with an equilibrium dissociation constant (K_{D}) of 0.1 nM, 0.2 nM, 0.3 nM, 0.4 nM, 0.5 nM, 0.6 nM, 0.7 nM, or 0.8 nM.

The equilibrium dissociation constant (K_{D}) as defined herein may be the ratio of the dissociation rate (K-off) and the association rate (K-on) of the anti-CD3 binding domain provided herein including embodiments thereof. It is described by the following formula: K_{D}=K-off/K-on. Thus, in embodiments, the anti-CD3 binding domain is capable of binding a CD3 protein with an equilibrium dissociation constant (K_{D}) of about 20 nM to about 60 nM. In embodiments, the anti-CD3 binding domain is capable of binding a CD3 protein with an equilibrium dissociation constant (K_{D}) of about 25 nM to about 60 nM. In embodiments, the anti-CD3 binding domain is capable of binding a CD3 protein with an equilibrium dissociation constant (K_{D}) of about 30 nM to about 60 nM. In embodiments, the anti-CD3 binding domain is capable of binding a CD3 protein with an equilibrium dissociation constant (K_{D}) of about 35 nM to about 60 nM. In embodiments, the anti-CD3 binding domain is capable of binding a CD3 protein with an equilibrium dissociation constant (K_{D}) of about 40 nM to about 60 nM. In embodiments, the anti-CD3 binding domain is capable of binding a CD3 protein with an equilibrium dissociation constant (K_{D}) of about 45 nM to about 60 nM. In embodiments, the anti-CD3 binding domain is capable of binding a CD3 protein with an equilibrium dissociation constant (K_{D}) of about 50 nM to about 60 nM. In embodiments, the anti-CD3 binding domain is capable of binding a CD3 protein with an equilibrium dissociation constant (K_{D}) of about 55 nM to about 60 nM.

In embodiments, the anti-CD3 binding domain is capable of binding a CD3 protein with an equilibrium dissociation constant (K_{D}) of 20 nM to 60 nM. In embodiments, the anti-CD3 binding domain is capable of binding a CD3 protein with an equilibrium dissociation constant (K_{D}) of 25 nM to 60 nM. In embodiments, the anti-CD3 binding domain is capable of binding a CD3 protein with an equilibrium dissociation constant (K_{D}) of 30 nM to 60 nM. In embodiments, the anti-CD3 binding domain is capable of binding a CD3 protein with an equilibrium dissociation constant (K_{D}) of 35 nM to 60 nM. In embodiments, the anti-CD3 binding domain is capable of binding a CD3 protein with an equilibrium dissociation constant (K_{D}) of 40 nM to 60 nM. In embodiments, the anti-CD3 binding domain is capable of binding a CD3 protein with an equilibrium dissociation constant (K_{D}) of 45 nM to 60 nM. In embodiments, the anti-CD3 binding domain is capable of binding a CD3 protein with an equilibrium dissociation constant (K_{D}) of 50 nM to 60 nM. In embodiments, the anti-CD3 binding domain is capable of binding a CD3 protein with an equilibrium dissociation constant (K_{D}) of 55 nM to 60 nM.

In embodiments, the anti-CD3 binding domain is capable of binding a CD3 protein with an equilibrium dissociation constant (K_{D}) of about 20 nM to about 55 nM. In embodiments, the anti-CD3 binding domain is capable of binding a CD3 protein with an equilibrium dissociation constant (K_{D}) of about 20 nM to about 50 nM. In embodiments, the anti-CD3 binding domain is capable of binding a CD3 protein with an equilibrium dissociation constant (K_{D}) of about 20 nM to about 45 nM. In embodiments, the anti-CD3 binding domain is capable of binding a CD3 protein with an equilibrium dissociation constant (K_{D}) of about 20 nM to about 40 nM. In embodiments, the anti-CD3 binding domain is capable of binding a CD3 protein with an equilibrium dissociation constant (K_{D}) of about 20 nM to about 35 nM. In embodiments, the anti-CD3 binding domain is capable of binding a CD3 protein with an equilibrium dissociation constant (K_{D}) of about 20 nM to about 30 nM. In embodiments, the anti-CD3 binding domain is capable of binding a CD3 protein with an equilibrium dissociation constant (K_{D}) of about 20 nM to about 25 nM.

In embodiments, the anti-CD3 binding domain is capable of binding a CD3 protein with an equilibrium dissociation constant (K_{D}) of 20 nM to 55 nM. In embodiments, the anti-CD3 binding domain is capable of binding a CD3 protein with an equilibrium dissociation constant (K_{D}) of 20 nM to 50 nM. In embodiments, the anti-CD3 binding domain is capable of binding a CD3 protein with an equilibrium dissociation constant (K_{D}) of 20 nM to 45 nM. In embodiments, the anti-CD3 binding domain is capable of binding a CD3 protein with an equilibrium dissociation constant (K_{D}) of 20 nM to 40 nM. In embodiments, the anti-CD3 binding domain is capable of binding a CD3 protein with an equilibrium dissociation constant (K_{D}) of 20 nM to 35 nM. In embodiments, the anti-CD3 binding domain is capable of binding a CD3 protein with an equilibrium dissociation constant (K_{D}) of 20 nM to 30 nM. In embodiments, the anti-CD3 binding domain is capable of binding a CD3 protein with an equilibrium dissociation constant (K_{D}) of 20 nM to 25 nM. In embodiments, the anti-CD3 binding domain is capable of binding a CD3 protein with an equilibrium dissociation constant (K_{D}) of about 20, 25, 30, 35, 40, 45, 50, 55, or 60 nM.

In embodiments, the anti-CD3 binding domain is capable of binding a CD3 protein with an equilibrium dissociation constant (K_{D}) of about 49 nM. In embodiments, the anti-CD3 binding domain is capable of binding a CD3 protein with an equilibrium dissociation constant (K_{D}) of 49 nM. In embodiments, the anti-CD3 binding domain is capable of binding a CD3 protein with an equilibrium dissociation constant (K_{D}) of about 36 nM. In embodiments, the anti-CD3 binding domain is capable of binding a CD3 protein with an equilibrium dissociation constant (K_{D}) of 36 nM.

In embodiments, the peptide provided herein forms part of a T cell. In embodiments, the peptide provided herein forms part of a natural killer cell (NK cell).

### Nucleic Acid Compositions

In an aspect is provided an isolated nucleic acid encoding a peptide as described herein, including embodiments thereof. The nucleic acid provided herein, including embodiments thereof, may be loaded into an expression vector such that the nucleic acid may be delivered to cells. Thus, in an aspect, an expression vector including the nucleic acid provided herein, including embodiments thereof, is provided. It is contemplated that the nucleic acid may be loaded into any expression vector useful for delivering the nucleic acid to cells either in vivo or in vitro. It is further contemplated that viruses, for example, lentivirus and onco-retrovirus, may serve as suitable expression vectors. Accordingly, in embodiments, the expression vector is a viral vector. In embodiments, the viral vector is a lentiviral vector or an onco-retroviral vector. In embodiments, the viral vector is a lentiviral vector. In embodiments, the viral vector is an onco-retroviral vector. In embodiments, the virus is a lentivirus or an onco-retrovirus. In embodiments, the virus is a lentivirus. In embodiments, the virus is an onco-retrovirus.

### Pharmaceutical Compositions

In an aspect is provided a pharmaceutical composition including a peptide as described herein, including embodiments thereof, and a pharmaceutically acceptable excipient.

### Methods of Treatment

The compositions provided herein, including embodiments thereof, are contemplated as providing effective treatments for diseases such as cancer. Thus, in an aspect is provided a method of treating cancer in a subject in need thereof. The method includes administering to a subject a therapeutically effective amount of a peptide provided herein, including embodiments thereof. In embodiments, the cancer is a CD38-expressing cancer. A “CD38-expressing cancer” as provided herein includes any cancer wherein the subject suffering from the cancer has one or more cancer cells that express CD38. In embodiments, the cancer is acute myelogenous leukemia (AML), multiple myeloma (MM), lymphoma, T-cell acute lymphoblastic leukemia (T-ALL), or prostate cancer. In embodiments, the cancer is acute myelogenous leukemia (AML). In embodiments, the cancer is multiple myeloma (MM). In embodiments, the cancer is lymphoma. In embodiments, the cancer is T-cell acute lymphoblastic leukemia (T-ALL). In embodiments, the cancer is prostate cancer. In embodiments, the cancer is a refractory cancer. In embodiments, the cancer is a chemo-resistant cancer. In embodiments, the subject includes Residual leukemia stem cells (LSCs). In embodiments, the LSCs are CD38 negative prior to the administering.

In embodiments, the peptide is administered at an amount that is less than the amount of an anti-cancer bispecific antibody normally administered to treat a cancer. In embodiments, the amount is 2, 5, 10, 100, 200, 500, 1000, or 10000 times less than the amount of an anti-cancer bispecific antibody. The term “anti-cancer bispecific antibody” refers to a bispecific molecule that is not a single chain polypeptide and which includes an effector domain (e.g., CD3 binding domain) and a target binding domain capable of binding a cancer-specific antigen (e.g., CD38). In embodiments, the anti-cancer bispecific antibody is a bispecific antibody including Daratumumab and a CD3 binding Fab domain.

In embodiments, the peptide is administered at an amount from about 0.5 mg/kg to about 20 mg/kg. In embodiments, the peptide is administered at an amount from about 1 mg/kg to about 20 mg/kg. In embodiments, the peptide is administered at an amount from about 1.5 mg/kg to about 20 mg/kg. In embodiments, the peptide is administered at an amount from about 2 mg/kg to about 20 mg/kg. In embodiments, the peptide is administered at an amount from about 2.5 mg/kg to about 20 mg/kg. In embodiments, the peptide is administered at an amount from about 3 mg/kg to about 20 mg/kg. In embodiments, the peptide is administered at an amount from about 3.5 mg/kg to about 20 mg/kg. In embodiments, the peptide is administered at an amount from about 4 mg/kg to about 20 mg/kg. In embodiments, the peptide is administered at an amount from about 4.5 mg/kg to about 20 mg/kg. In embodiments, the peptide is administered at an amount from about 5 mg/kg to about 20 mg/kg. In embodiments, the peptide is administered at an amount from about 5.5 mg/kg to about 20 mg/kg. In embodiments, the peptide is administered at an amount from about 6 mg/kg to about 20 mg/kg. In embodiments, the peptide is administered at an amount from about 6.5 mg/kg to about 20 mg/kg. In embodiments, the peptide is administered at an amount from about 7 mg/kg to about 20 mg/kg. In embodiments, the peptide is administered at an amount from about 7.5 mg/kg to about 20 mg/kg. In embodiments, the peptide is administered at an amount from about 8 mg/kg to about 20 mg/kg. In embodiments, the peptide is administered at an amount from about 8.5 mg/kg to about 20 mg/kg. In embodiments, the peptide is administered at an amount from about 9 mg/kg to about 20 mg/kg. In embodiments, the peptide is administered at an amount from about 9.5 mg/kg to about 20 mg/kg. In embodiments, the peptide is administered at an amount from about 10 mg/kg to about 20 mg/kg.

In embodiments, the peptide is administered at an amount from about 10.5 mg/kg to about 20 mg/kg. In embodiments, the peptide is administered at an amount from about 11 mg/kg to about 20 mg/kg. In embodiments, the peptide is administered at an amount from about 11.5 mg/kg to about 20 mg/kg. In embodiments, the peptide is administered at an amount from about 12 mg/kg to about 20 mg/kg. In embodiments, the peptide is administered at an amount from about 12.5 mg/kg to about 20 mg/kg. In embodiments, the peptide is administered at an amount from about 13 mg/kg to about 20 mg/kg. In embodiments, the peptide is administered at an amount from about 13.5 mg/kg to about 20 mg/kg. In embodiments, the peptide is administered at an amount from about 14 mg/kg to about 20 mg/kg. In embodiments, the peptide is administered at an amount from about 14.5 mg/kg to about 20 mg/kg. In embodiments, the peptide is administered at an amount from about 15 mg/kg to about 20 mg/kg. In embodiments, the peptide is administered at an amount from about 15.5 mg/kg to about 20 mg/kg. In embodiments, the peptide is administered at an amount from about 6 mg/kg to about 20 mg/kg. In embodiments, the peptide is administered at an amount from about 16.5 mg/kg to about 20 mg/kg. In embodiments, the peptide is administered at an amount from about 17 mg/kg to about 20 mg/kg. In embodiments, the peptide is administered at an amount from about 17.5 mg/kg to about 20 mg/kg. In embodiments, the peptide is administered at an amount from about 18 mg/kg to about 20 mg/kg. In embodiments, the peptide is administered at an amount from about 18.5 mg/kg to about 20 mg/kg. In embodiments, the peptide is administered at an amount from about 19 mg/kg to about 20 mg/kg. In embodiments, the peptide is administered at an amount from about 19.5 mg/kg to about 20 mg/kg. In embodiments, the peptide is administered at an amount of about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg.

In embodiments, the peptide is administered at an amount from 0.5 mg/kg to 20 mg/kg. In embodiments, the peptide is administered at an amount from 1 mg/kg to 20 mg/kg. In embodiments, the peptide is administered at an amount from 1.5 mg/kg to 20 mg/kg. In embodiments, the peptide is administered at an amount from 2 mg/kg to 20 mg/kg. In embodiments, the peptide is administered at an amount from 2.5 mg/kg to 20 mg/kg. In embodiments, the peptide is administered at an amount from 3 mg/kg to 20 mg/kg. In embodiments, the peptide is administered at an amount from 3.5 mg/kg to 20 mg/kg. In embodiments, the peptide is administered at an amount from 4 mg/kg to 20 mg/kg. In embodiments, the peptide is administered at an amount from 4.5 mg/kg to 20 mg/kg. In embodiments, the peptide is administered at an amount from 5 mg/kg to 20 mg/kg. In embodiments, the peptide is administered at an amount from 5.5 mg/kg to 20 mg/kg. In embodiments, the peptide is administered at an amount from 6 mg/kg to 20 mg/kg. In embodiments, the peptide is administered at an amount from 6.5 mg/kg to 20 mg/kg. In embodiments, the peptide is administered at an amount from 7 mg/kg to 20 mg/kg. In embodiments, the peptide is administered at an amount from 7.5 mg/kg to 20 mg/kg. In embodiments, the peptide is administered at an amount from 8 mg/kg to 20 mg/kg. In embodiments, the peptide is administered at an amount from 8.5 mg/kg to 20 mg/kg. In embodiments, the peptide is administered at an amount from 9 mg/kg to 20 mg/kg. In embodiments, the peptide is administered at an amount from 9.5 mg/kg to 20 mg/kg. In embodiments, the peptide is administered at an amount from 10 mg/kg to 20 mg/kg.

In embodiments, the peptide is administered at an amount from 10.5 mg/kg to 20 mg/kg. In embodiments, the peptide is administered at an amount from 11 mg/kg to 20 mg/kg. In embodiments, the peptide is administered at an amount from 11.5 mg/kg to 20 mg/kg. In embodiments, the peptide is administered at an amount from 12 mg/kg to 20 mg/kg. In embodiments, the peptide is administered at an amount from 12.5 mg/kg to 20 mg/kg. In embodiments, the peptide is administered at an amount from 13 mg/kg to 20 mg/kg. In embodiments, the peptide is administered at an amount from 13.5 mg/kg to 20 mg/kg. In embodiments, the peptide is administered at an amount from 14 mg/kg to 20 mg/kg. In embodiments, the peptide is administered at an amount from 14.5 mg/kg to 20 mg/kg. In embodiments, the peptide is administered at an amount from 15 mg/kg to 20 mg/kg. In embodiments, the peptide is administered at an amount from 15.5 mg/kg to 20 mg/kg. In embodiments, the peptide is administered at an amount from 6 mg/kg to 20 mg/kg. In embodiments, the peptide is administered at an amount from 16.5 mg/kg to 20 mg/kg. In embodiments, the peptide is administered at an amount from 17 mg/kg to 20 mg/kg. In embodiments, the peptide is administered at an amount from 17.5 mg/kg to 20 mg/kg. In embodiments, the peptide is administered at an amount from 18 mg/kg to 20 mg/kg. In embodiments, the peptide is administered at an amount from 18.5 mg/kg to 20 mg/kg. In embodiments, the peptide is administered at an amount from 19 mg/kg to 20 mg/kg. In embodiments, the peptide is administered at an amount from 19.5 mg/kg to 20 mg/kg. In embodiments, the peptide is administered at an amount of 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg. The term “mg/kg” as provided herein refers to the mg of peptide or salt thereof per kg body weight.

In embodiments, the peptide is administered at an amount from about 0.5 mg/kg to about 19.5 mg/kg. In embodiments, the peptide is administered at an amount from about 0.5 mg/kg to about 19 mg/kg. In embodiments, the peptide is administered at an amount from about 0.5 mg/kg to about 18.5 mg/kg. In embodiments, the peptide is administered at an amount from about 0.5 mg/kg to about 18 mg/kg. In embodiments, the peptide is administered at an amount from about 0.5 mg/kg to about 17.5 mg/kg. In embodiments, the peptide is administered at an amount from about 0.5 mg/kg to about 17 mg/kg. In embodiments, the peptide is administered at an amount from about 0.5 mg/kg to about 16.5 mg/kg. In embodiments, the peptide is administered at an amount from about 0.5 mg/kg to about 16 mg/kg. In embodiments, the peptide is administered at an amount from about 0.5 mg/kg to about 15.5 mg/kg. In embodiments, the peptide is administered at an amount from about 0.5 mg/kg to about 15 mg/kg. In embodiments, the peptide is administered at an amount from about 0.5 mg/kg to about 14.5 mg/kg. In embodiments, the peptide is administered at an amount from about 0.5 mg/kg to about 14 mg/kg. In embodiments, the peptide is administered at an amount from about 0.5 mg/kg to about 13.5 mg/kg. In embodiments, the peptide is administered at an amount from about 0.5 mg/kg to about 13 mg/kg. In embodiments, the peptide is administered at an amount from about 0.5 mg/kg to about 12.5 mg/kg. In embodiments, the peptide is administered at an amount from about 0.5 mg/kg to about 12 mg/kg. In embodiments, the peptide is administered at an amount from about 0.5 mg/kg to about 11.5 mg/kg. In embodiments, the peptide is administered at an amount from about 0.5 mg/kg to about 11 mg/kg. In embodiments, the peptide is administered at an amount from about 0.5 mg/kg to about 10.5 mg/kg. In embodiments, the peptide is administered at an amount from about 0.5 mg/kg to about 10 mg/kg.

In embodiments, the peptide is administered at an amount from about 0.5 mg/kg to about 9.5 mg/kg. In embodiments, the peptide is administered at an amount from about 0.5 mg/kg to about 9 mg/kg. In embodiments, the peptide is administered at an amount from about 0.5 mg/kg to about 8.5 mg/kg. In embodiments, the peptide is administered at an amount from about 0.5 mg/kg to about 8 mg/kg. In embodiments, the peptide is administered at an amount from about 0.5 mg/kg to about 7.5 mg/kg. In embodiments, the peptide is administered at an amount from about 0.5 mg/kg to about 7 mg/kg. In embodiments, the peptide is administered at an amount from about 0.5 mg/kg to about 6.5 mg/kg. In embodiments, the peptide is administered at an amount from about 0.5 mg/kg to about 6 mg/kg. In embodiments, the peptide is administered at an amount from about 0.5 mg/kg to about 5.5 mg/kg. In embodiments, the peptide is administered at an amount from about 0.5 mg/kg to about 5 mg/kg. In embodiments, the peptide is administered at an amount from about 0.5 mg/kg to about 4.5 mg/kg. In embodiments, the peptide is administered at an amount from about 0.5 mg/kg to about 4 mg/kg. In embodiments, the peptide is administered at an amount from about 0.5 mg/kg to about 3.5 mg/kg. In embodiments, the peptide is administered at an amount from about 0.5 mg/kg to about 3 mg/kg. In embodiments, the peptide is administered at an amount from about 0.5 mg/kg to about 2.5 mg/kg. In embodiments, the peptide is administered at an amount from about 0.5 mg/kg to about 2 mg/kg. In embodiments, the peptide is administered at an amount from about 0.5 mg/kg to about 1.5 mg/kg. In embodiments, the peptide is administered at an amount from about 0.5 mg/kg to about 1 mg/kg.

In embodiments, the peptide is administered at an amount from 0.5 mg/kg to 19.5 mg/kg. In embodiments, the peptide is administered at an amount from 0.5 mg/kg to 19 mg/kg. In embodiments, the peptide is administered at an amount from 0.5 mg/kg to 18.5 mg/kg. In embodiments, the peptide is administered at an amount from 0.5 mg/kg to 18 mg/kg. In embodiments, the peptide is administered at an amount from 0.5 mg/kg to 17.5 mg/kg. In embodiments, the peptide is administered at an amount from 0.5 mg/kg to 17 mg/kg. In embodiments, the peptide is administered at an amount from 0.5 mg/kg to 16.5 mg/kg. In embodiments, the peptide is administered at an amount from 0.5 mg/kg to 16 mg/kg. In embodiments, the peptide is administered at an amount from 0.5 mg/kg to 15.5 mg/kg. In embodiments, the peptide is administered at an amount from 0.5 mg/kg to 15 mg/kg. In embodiments, the peptide is administered at an amount from 0.5 mg/kg to 14.5 mg/kg. In embodiments, the peptide is administered at an amount from 0.5 mg/kg to 14 mg/kg. In embodiments, the peptide is administered at an amount from 0.5 mg/kg to 13.5 mg/kg. In embodiments, the peptide is administered at an amount from 0.5 mg/kg to 13 mg/kg. In embodiments, the peptide is administered at an amount from 0.5 mg/kg to 12.5 mg/kg. In embodiments, the peptide is administered at an amount from 0.5 mg/kg to 12 mg/kg. In embodiments, the peptide is administered at an amount from 0.5 mg/kg to 11.5 mg/kg. In embodiments, the peptide is administered at an amount from 0.5 mg/kg to 11 mg/kg. In embodiments, the peptide is administered at an amount from 0.5 mg/kg to 10.5 mg/kg. In embodiments, the peptide is administered at an amount from 0.5 mg/kg to 10 mg/kg.

In embodiments, the peptide is administered at an amount from 0.5 mg/kg to 9.5 mg/kg. In embodiments, the peptide is administered at an amount from 0.5 mg/kg to 9 mg/kg. In embodiments, the peptide is administered at an amount from 0.5 mg/kg to 8.5 mg/kg. In embodiments, the peptide is administered at an amount from 0.5 mg/kg to 8 mg/kg. In embodiments, the peptide is administered at an amount from 0.5 mg/kg to 7.5 mg/kg. In embodiments, the peptide is administered at an amount from 0.5 mg/kg to 7 mg/kg. In embodiments, the peptide is administered at an amount from 0.5 mg/kg to 6.5 mg/kg. In embodiments, the peptide is administered at an amount from 0.5 mg/kg to 6 mg/kg. In embodiments, the peptide is administered at an amount from 0.5 mg/kg to 5.5 mg/kg. In embodiments, the peptide is administered at an amount from 0.5 mg/kg to 5 mg/kg. In embodiments, the peptide is administered at an amount from 0.5 mg/kg to 4.5 mg/kg. In embodiments, the peptide is administered at an amount from 0.5 mg/kg to 4 mg/kg. In embodiments, the peptide is administered at an amount from 0.5 mg/kg to 3.5 mg/kg. In embodiments, the peptide is administered at an amount from 0.5 mg/kg to 3 mg/kg. In embodiments, the peptide is administered at an amount from 0.5 mg/kg to 2.5 mg/kg. In embodiments, the peptide is administered at an amount from 0.5 mg/kg to 2 mg/kg. In embodiments, the peptide is administered at an amount from 0.5 mg/kg to 1.5 mg/kg. In embodiments, the peptide is administered at an amount from 0.5 mg/kg to 1 mg/kg.

### Methods of Detecting

The compositions provided herein, including embodiments thereof, are contemplated as diagnostic tools for detecting cancer in vivo. Thus, in an aspect is provided a method of detecting a cancer cell in a subject in need thereof the method including administering to a subject in need thereof a peptide as described herein, including embodiments thereof and a detecting agent. In embodiments, the agent is a labeled peptide. In embodiments, the labeled peptide is bound by the peptide provided herein including embodiments thereof thereby detecting the cancer in said subject.

In embodiments, the peptide includes a detectable moiety. In embodiments, the detectable moiety is bound (covalently or non-covalently) to the anti-CD3 binding domain. In embodiments, the anti-CD3 binding domain includes a detectable moiety.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

Applicants used bionic technology to create a single chain anti-CD38/anti-CD3 Bispecific T cell Engager (Bite). Exemplary experiments described herein illustrate that CD38 and CD3 moieties retain binding affinity in this bispecific format. Results show that the bispecific T cell engagers may activate T cells in the presence of tumor antigen presenting cells.

### Example 1: Generation of Single Chain Bispecific Compounds

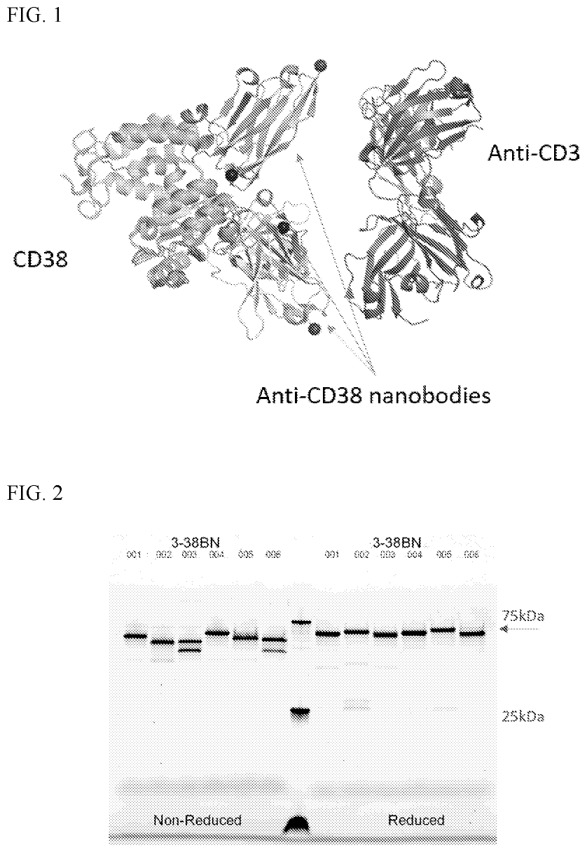

Bispecific antibody compounds were created by connecting antibody single chains. Specifically, a heavy chain of an anti-CD3 antibody was connected to an anti-CD38 nanobody with a linker. Using a second linker, the light chain of an anti-CD3 antibody was connected to the anti-CD38 nanobody. This second linker includes a tumor specific protease cleavage site, for example a metalloprotease 9 (MMP-9) substrate cleavage site. Cleavage of this protease substrate frees the anti-CD3 chain, thus activating the bispecific T cell engager and allowing the anti-CD3 to bind its target (FIG. 1). Table 1 shows single-chain bispecific compounds and linker sequences. The bold residues show the MMP-9 cleavage site.

TABLE 1
Single Chain Bispecific Compounds

| | | Linker1 | Linker2 |
|---|---|---|---|
| 3- | CD3HC-L1- | AASGGAGS | GAVPLSLYGAGG |
| 38BN001 | CD38nanobody | | |
| | (5F1K)-L2- | | |
| | CD3LC | | |
| 3- | CD3HC-L1- | AASGGAGS | GAVPLSLYGAGG |
| 38BN002 | CD38nanobody | | |
| | (5F1O)-L2- | | |
| | CD3LC | | |
| 3- | CD3HC-L1- | AASGGAGS | GAVPLSLYGAGG |
| 38BN003 | CD38nanobody | | |
| | (5F21)-L2- | | |
| | CD3LC | | |
| 3- | CD3HC-L1- | AASGGAGS | SSGASASAAGGS |
| 38BN004 | CD38nanobody | | |
| | (5F1K)-L2- | | |
| | CD3LC | | |
| 3- | CD3HC-L1- | AASGGAGS | SSGASASAAGGS |
| 38BN005 | CD38nanobody | | |
| | (5F1O)-L2- | | |
| | CD3LC | | |
| 3- | CD3HC-L1- | AASGGAGS | SSGASASAAGGS |
| 38BN006 | CD38nanobody | | |
| | (5F21)-L2- | | |
| | CD3LC | | |

The single-chain bispecific compounds were purified, and were subsequently characterized by non-reducing native-PAGE and reducing SDS-PAGE gels (FIG. 2).

### Example 2: CD3-CD8 Bispecific Compound Retains Binding Ability to CD38

The bispecific compounds were assessed for their binding ability to CD38 using surface plasmon resonance (SPR). CD38-Fc was immobilized on a CM5 sensor chip at 200 response units (RU) using ECD/NHS coupling. Samples of bispecific compounds 38BN001, 38BN002, and 38BN003 were prepared either with or without Matrix metalloproteinase-1 (MMP-1). These bispecific compounds include a protease cleavage site within one of the linker regions. Samples of the bispecific compounds 38BN004, 38BN005, and 38BN006 were prepared without MMP-1, as these compounds do not include a protease cleavage site. Anti-CD38 antibody (Daratumumab) was used as a positive control and anti-HER2 antibody (Trastuzumab) as a negative control. All samples were prepared at concentrations of 30 nM, 10 nM, 3 nM or 1 nM in HBS-EP+ runner buffer at 25° C.

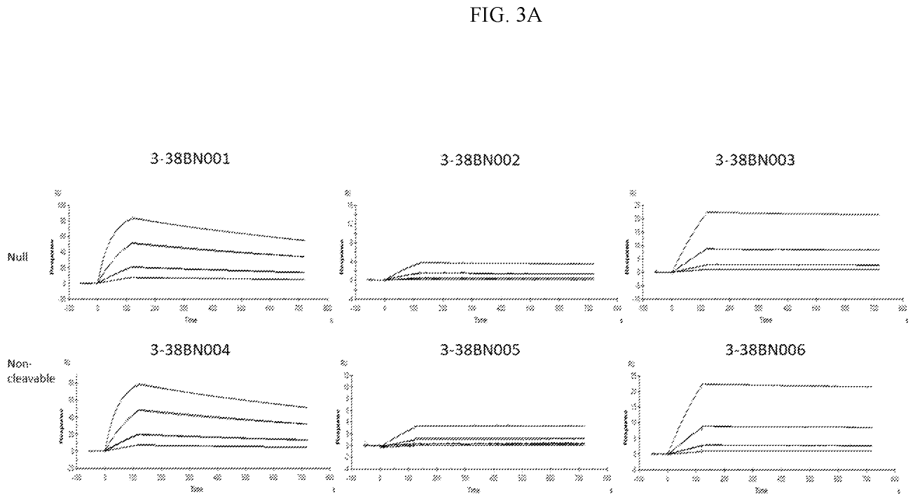

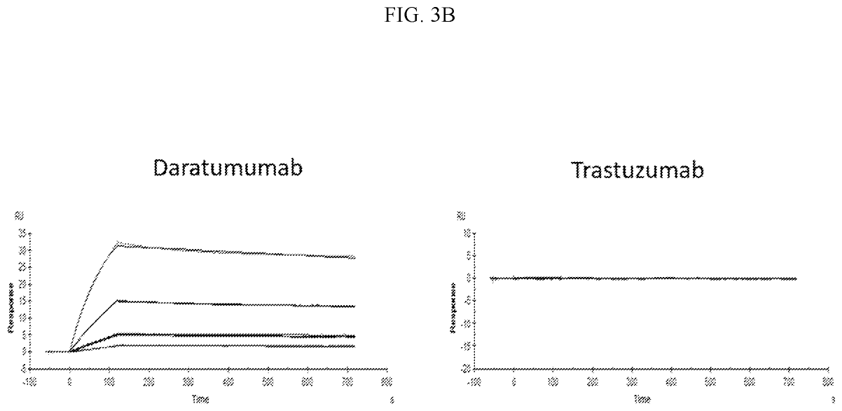

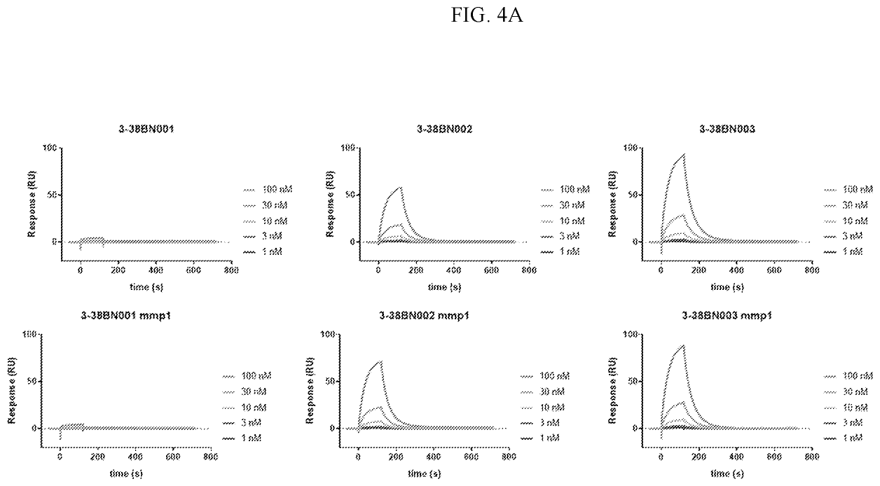

Results show that the CD38 nanobody, in both cleavable and non-cleavable bispecific formats, are able to bind CD38 antigen target (FIG. 3A). As expected, Daratumumab bound the target with high affinity while Trastuzumab did not bind (FIG. 3B). Further, 38BN003 and 38N006 displayed highest change in response units, while 38BN002 and 38BN005 showed the lowest change in response units. Table 2 shows binding properties of the bispecific antibody compounds to the CD38 target. “*” indicates IgG and “**” indicates the kinetic constant kd is outside of detectable limits.

TABLE 2
Binding properties of bispecific compounds to CD38

| | Ka (1/Ms) | kd (1/s) | KD (M) | Chi²(RU²) | U-value |
|---|---|---|---|---|---|
| 3-38BN001 null | 7.352E+5 | 6.956E−4 | 9.46E−10 | 0.318 | 1 |
| 3-38BN002 null | 1.647E+5 | 1.272E−4 | 7.72E−10 | 0.0158 | 15 |
| 3-38BN003 null | 1.428E+5 | 6.836E−5 | 4.78E−10 | 0.0208 | 5 |
| 3-38BN004 null | 7.398E+5 | 7.114E−4 | 9.62E−10 | 0.490 | 1 |
| 3-38BN005 null** | 5.896E+5 | 2.113E−6 | 3.58E−10 | 0.00815 | 95 |
| 3-38BN006 null | 1.547E+5 | 7.325E−4 | 4.735E−10 | 0.0138 | 4 |
| Daratumumab* | 3.732E+5 | 2.043E−4 | 5.47E−10 | 0.0591 | 1 |

| | |
|---|---|
| Trastuzumab | ND |

### Example 3: CD3-CD38 Bispecific Compound Retains Binding Ability to CD3

Using SPR, the bispecific compounds were assessed for their binding ability to CD3. CD3-Fc was immobilized on a CM5 sensor chip at 1000 response units (RU) using ECD/NHS coupling methods. Samples of bispecific compounds 38BN001, 38BN002, and 38BN003 were prepared either with or without MMP-1, and samples of bispecific compounds 38BN004, 38BN005, and 38BN006 were prepared without MMP-1. Anti-CD3 Fab was used as a positive control and the Trastuzumab Fab as a negative control. All samples were prepared at concentrations of 30 nM, 10 nM, 3 nM or 1 nM in HBS-EP+ runner buffer at 25° C.

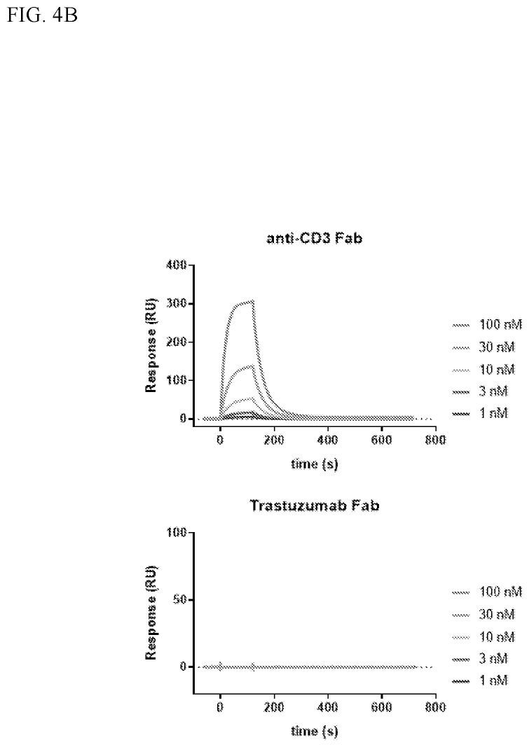

Results illustrate that with or without MMP1, the 38N002 and 38N003 compounds bind to their CD3 target (FIG. 4A). Table 3 shows binding properties of the bispecific compounds to the CD3 target.

TABLE 3
Binding properties of bispecific compounds to CD3

| | ka | kd | KD | Chi² | |
|---|---|---|---|---|---|
| | (1/Ms) | (1/s) | (M) | (RU²) | U-value |
| 3-38BN001 null | ND | | | | |
| 3-38BN002 null | 4.29E+04 | 2.27E−02 | 5.30E−07 | 0.165 | 9 |
| 3-38BN003 null | 1.01E+05 | 0.05252 | 5.22E−07 | 0.326 | 5 |
| Anti-CD3 Fab | 4.10E+05 | 0.03825 | 9.33E−08 | 3.05 | 1 |

### Example 4: CD3-CD38 Bispecific Compounds Bind Target Cells and Mediate Antibody Dependent Cytotoxicity

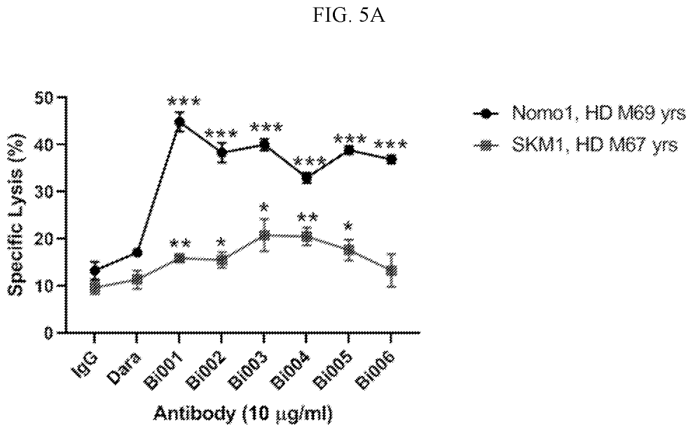

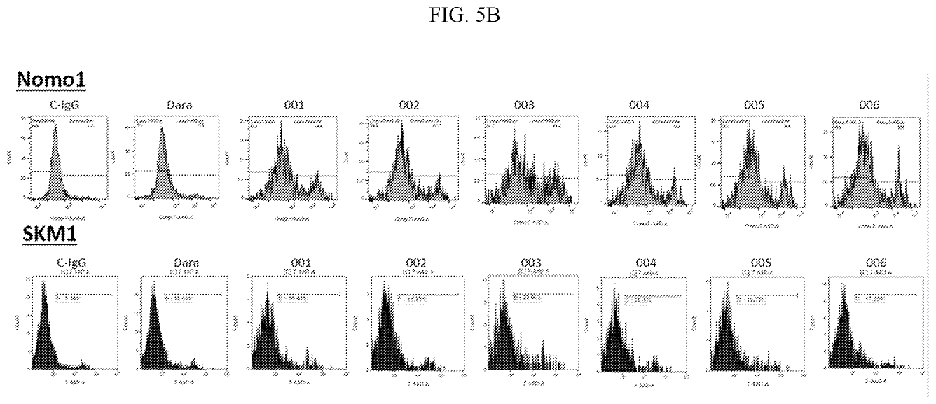

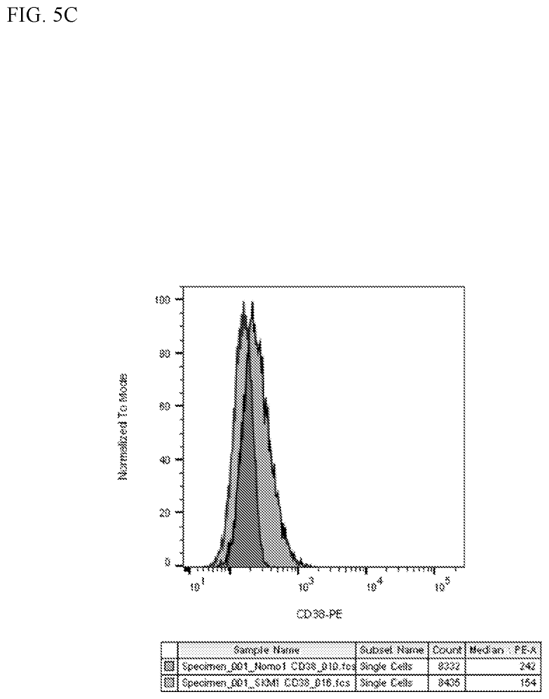

Nomo-1 cells, an AML cell line which presents high levels of CD38, were incubated with the CD3-CD38 bispecific compounds and effector cells. SKM-1 AML cells, which express low levels of CD38 were similarly incubated with the bispecific compounds and effector cells. Results show that the bispecific T cell engagers activate T cells in the presence of tumor antigen presenting cells, as illustrated by the higher percentage of lysis of Nomo-1 cells as compared to SKM-1 cells (FIG. 5A). Binding of the bispecific compounds was further demonstrated by flow cytometry analysis, which showed that the compounds bind to Nomo-1 cells to a higher extent than SKM-1 cells (FIGS. 5B and 5C).

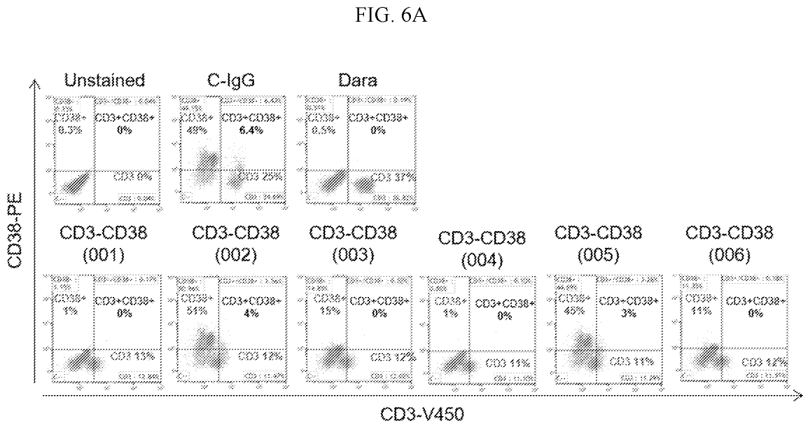

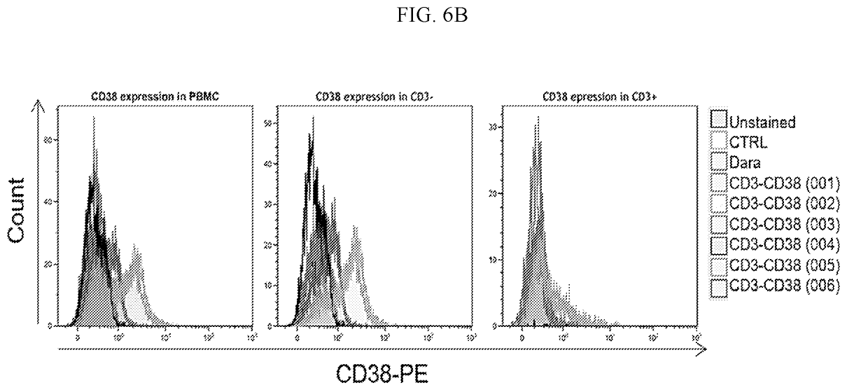

The CD3-CD38 compounds were then incubated with PBMCs to assess binding to CD3 and CD38 antigens presented on effector cells. Results illustrate that the compounds bind both CD3 and CD38, as assessed by flow cytometry (FIG. 6A).

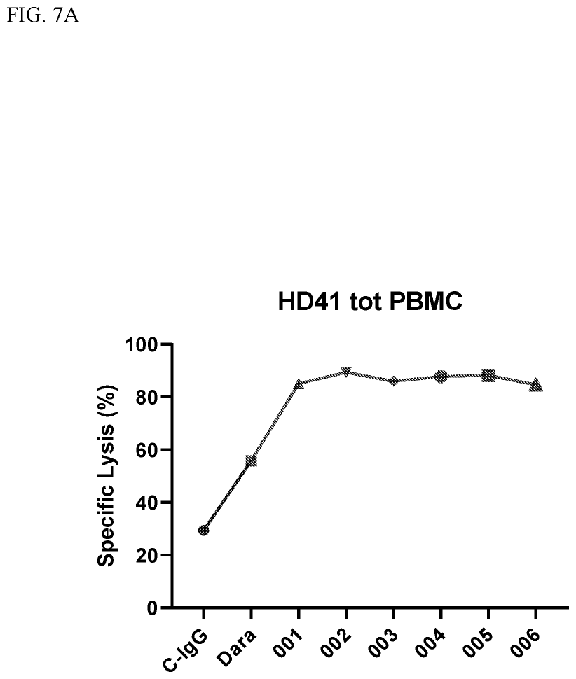

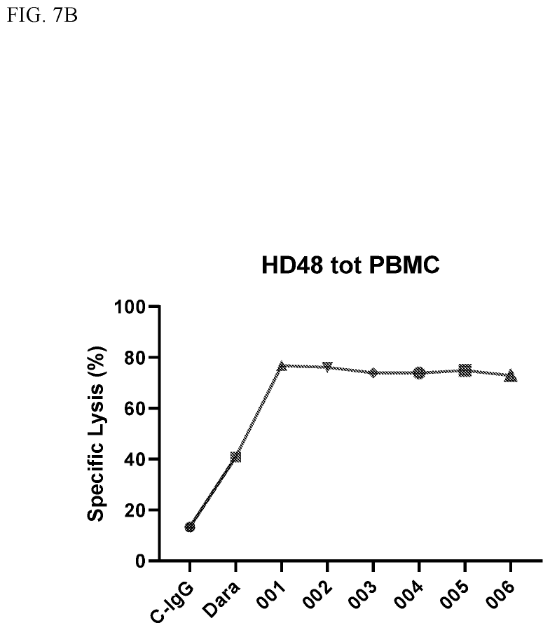

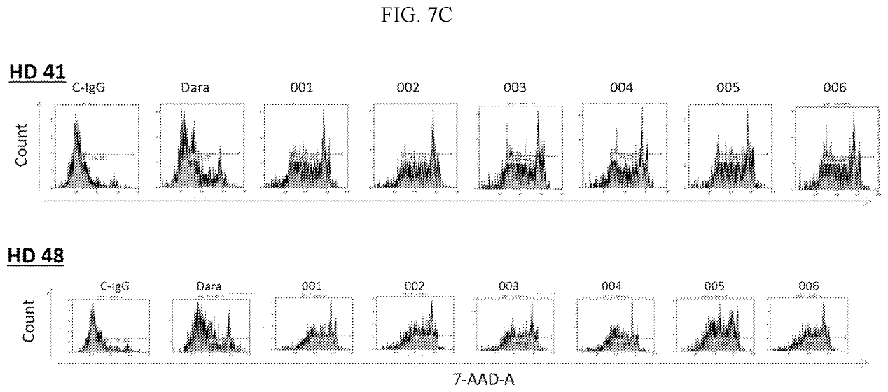

The CD3-CD38 bispecific compounds were evaluated for their ability to mediate ADCC in CD38+ multiple myeloma cells. MMIS cells, which expressed green fluorescent protein (GFP), were treated with bispecific compounds and effector cells HD41 PBMC or HD48 PBMCs. Results show that all six bispecific compounds 38BN001, 38BN002, 38BN003, 38BN004, 38BN005, and 38BN006 activate the effector cells to induce ADCC of the MMIS target cells (FIGS. 7A and 7B). Further, flow cytometry analysis confirmed that the bispecific antibodies bind to antigens presented on the effector cell surface (FIG. 7C).

### Example 5: CD38 and CD3 Bionics

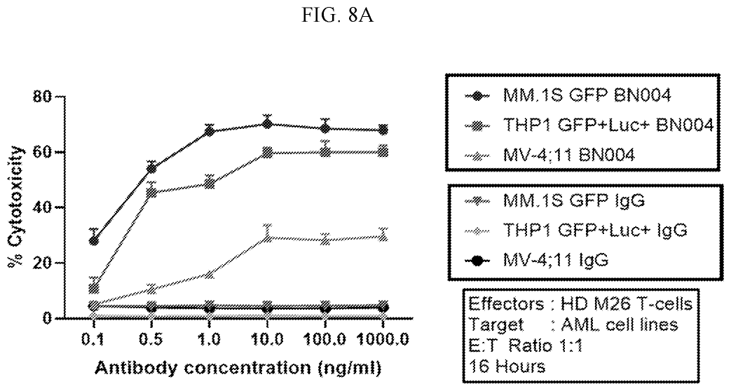

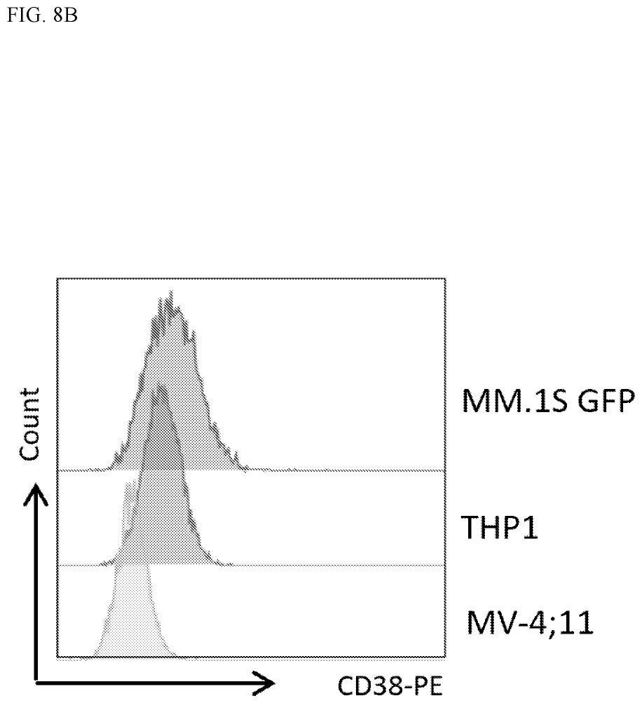

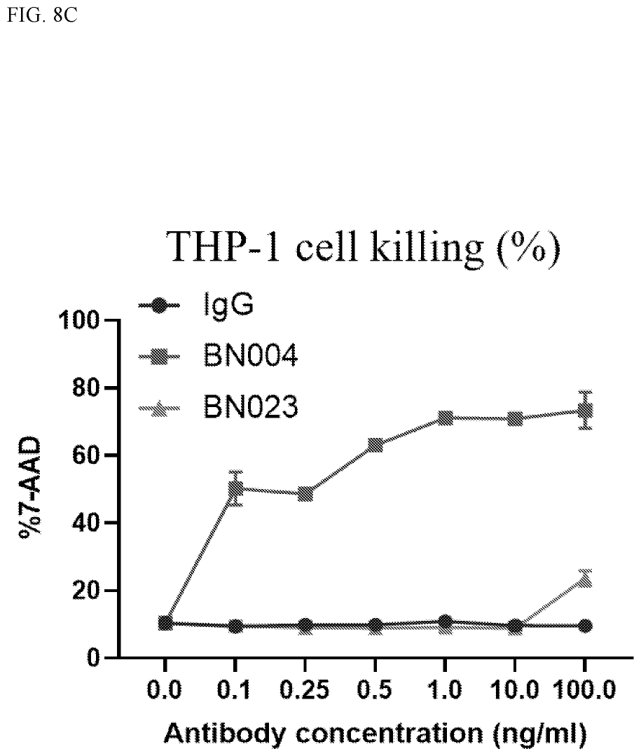

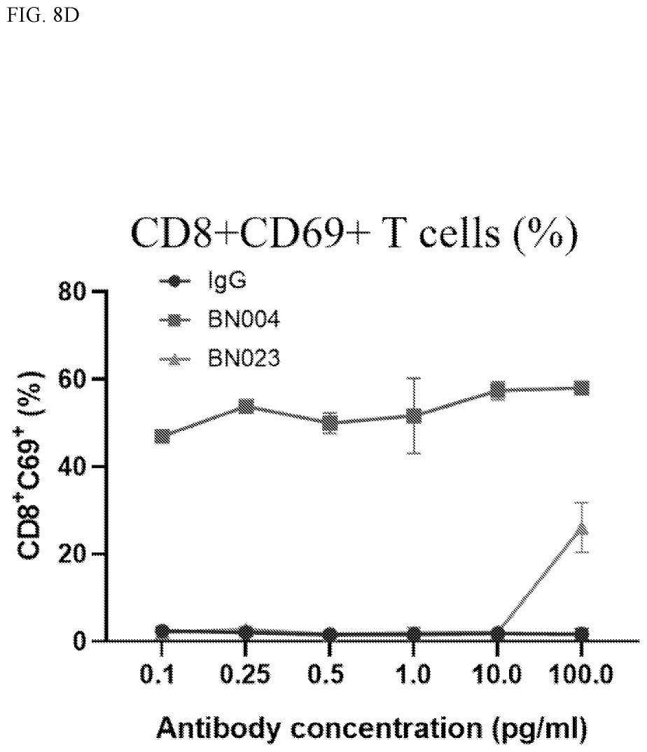

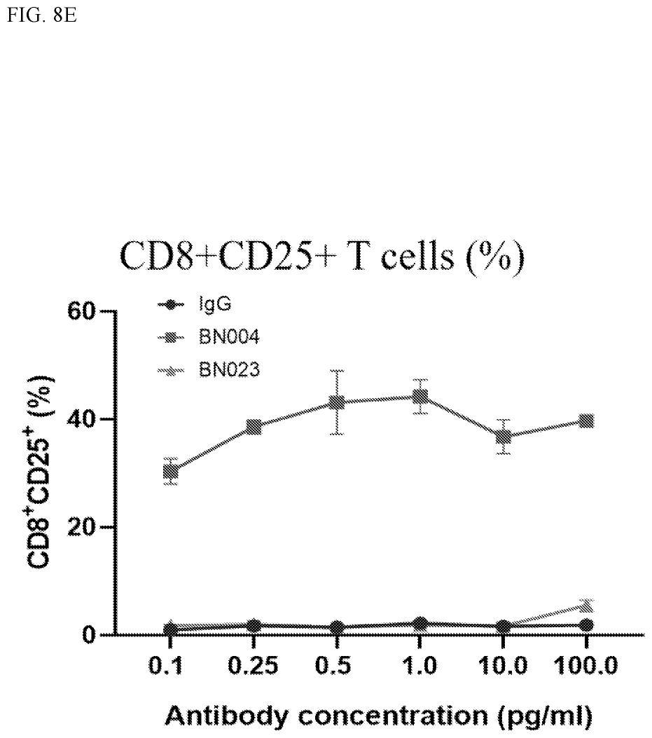

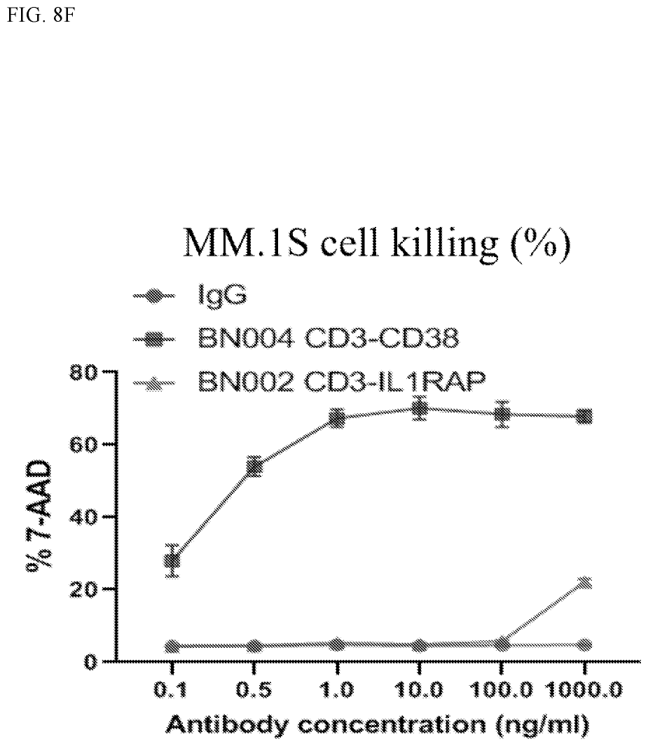

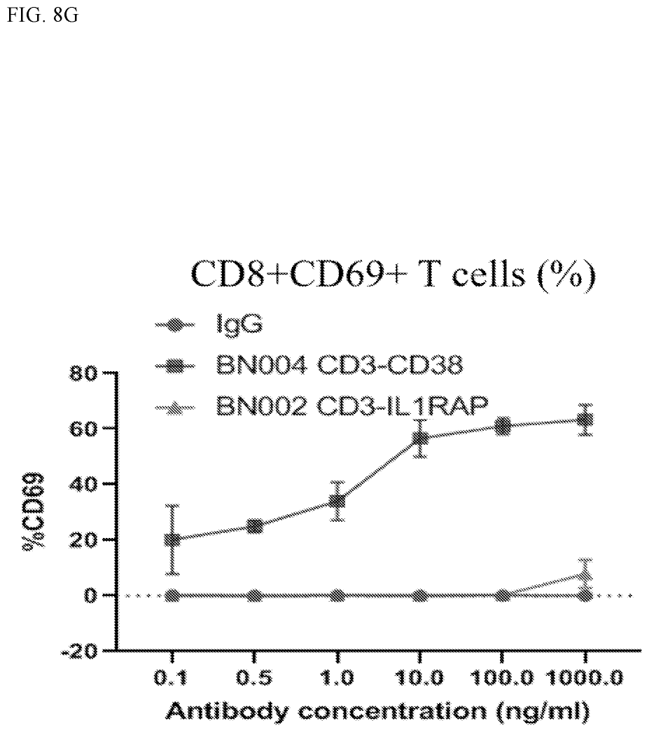

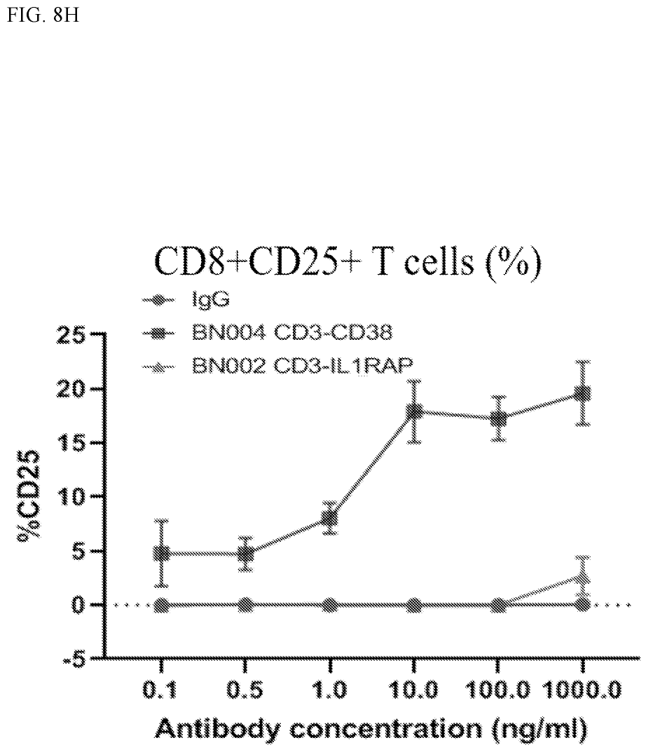

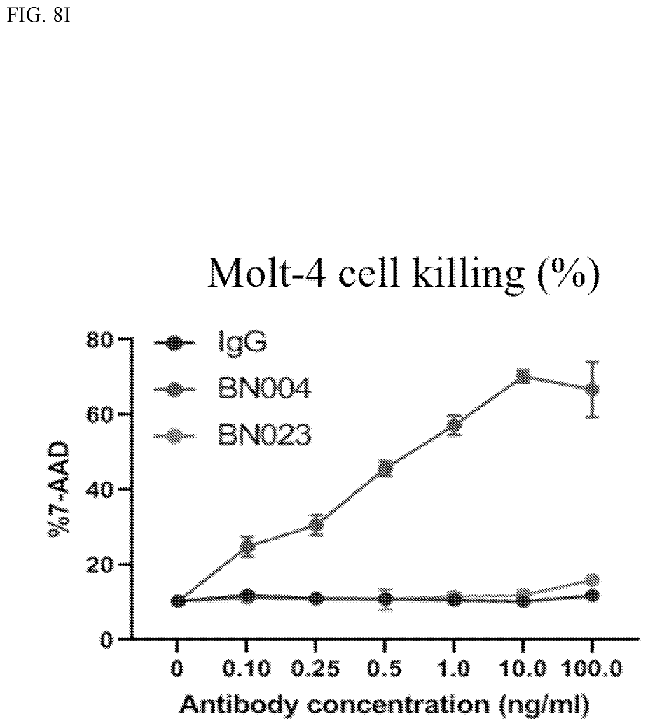

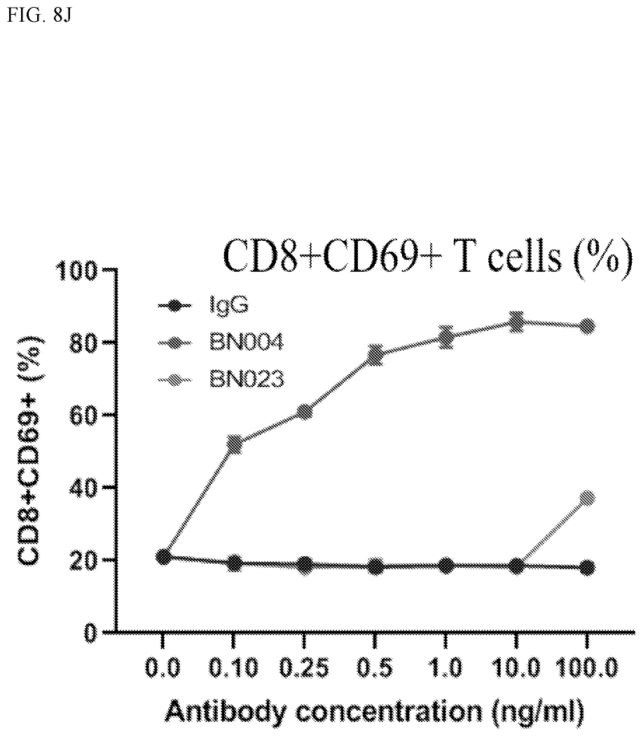

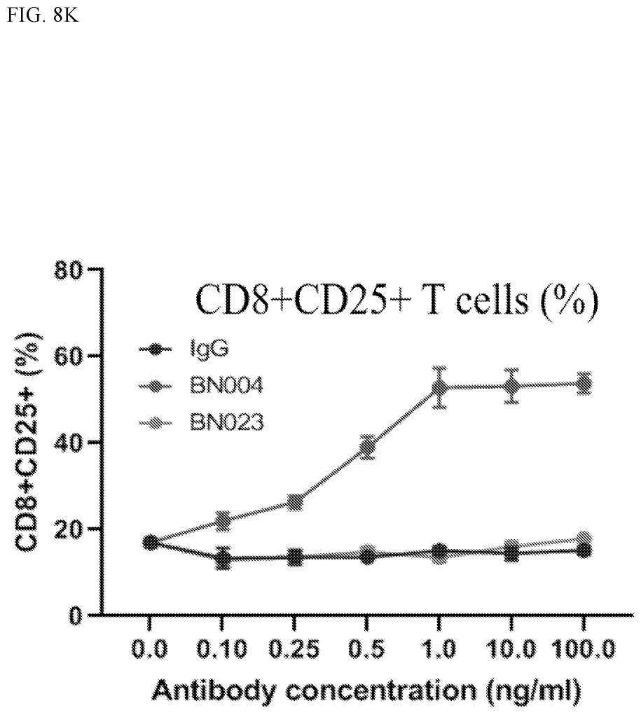

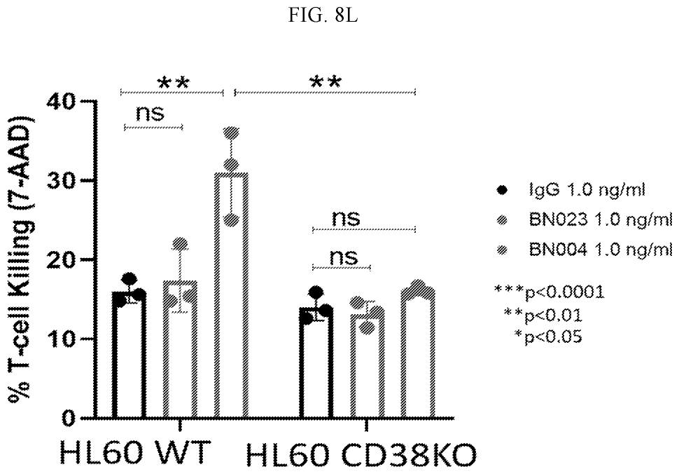

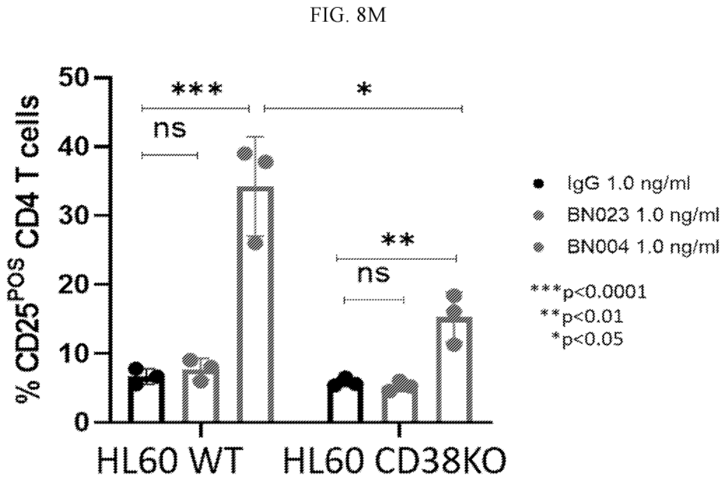

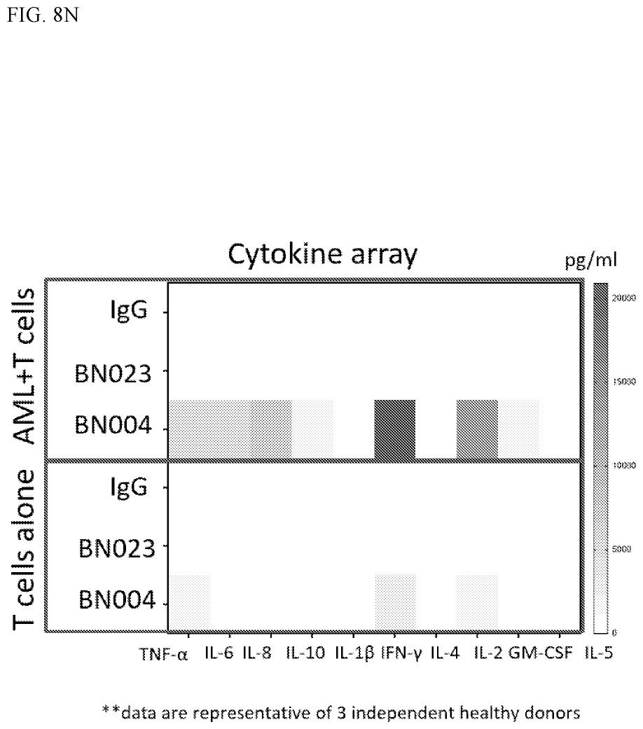

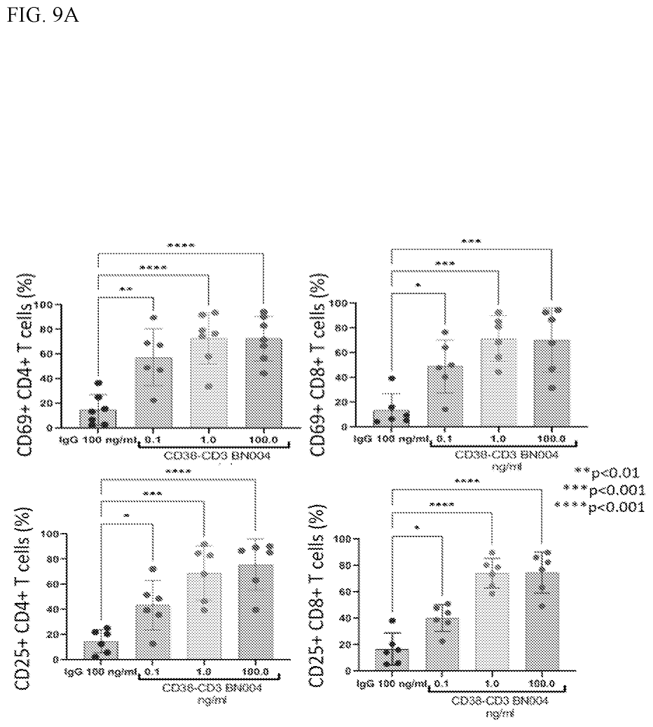

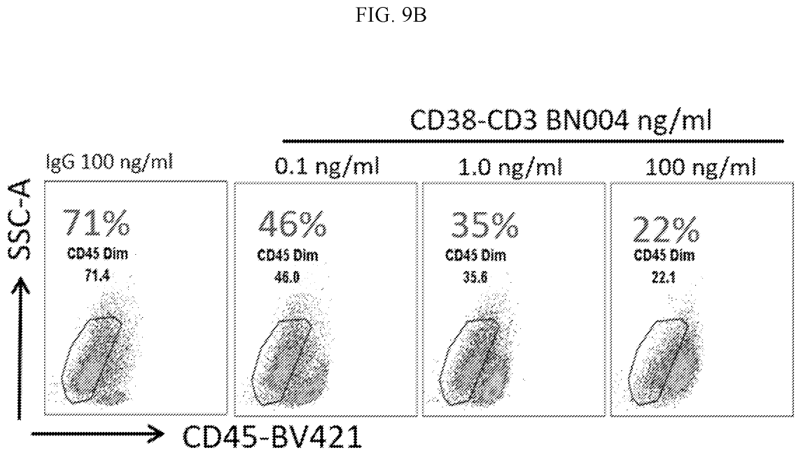

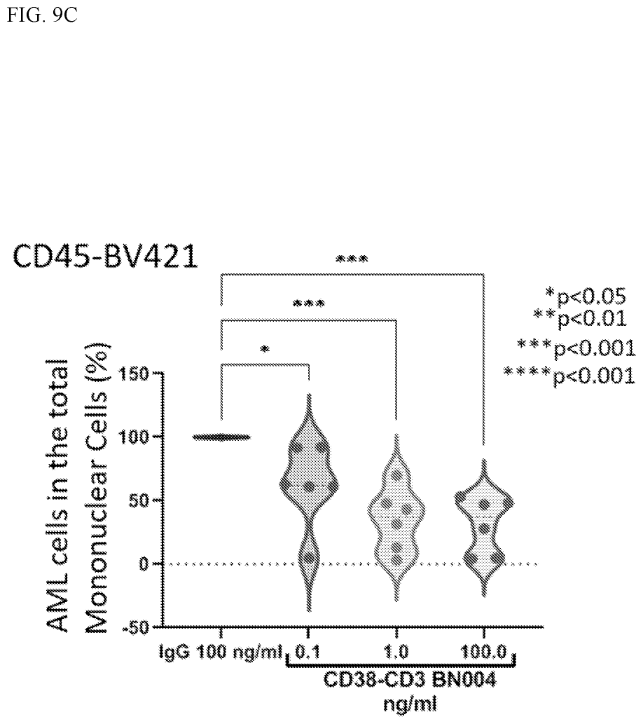

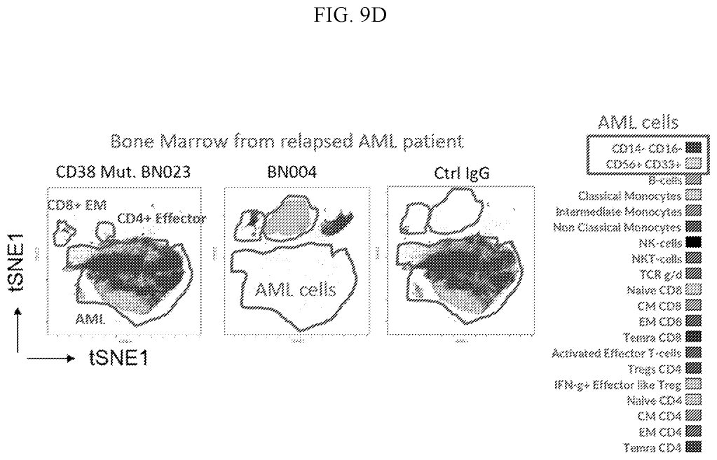

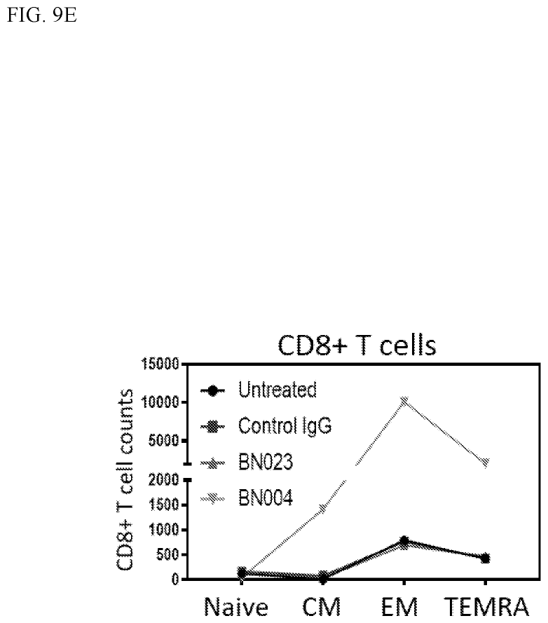

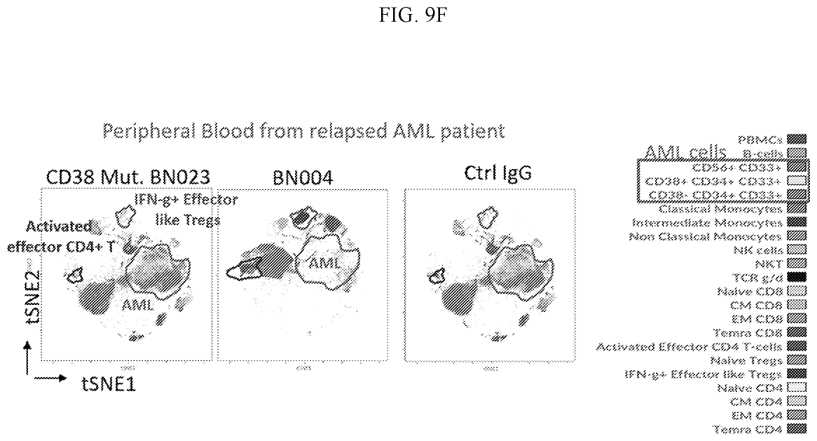

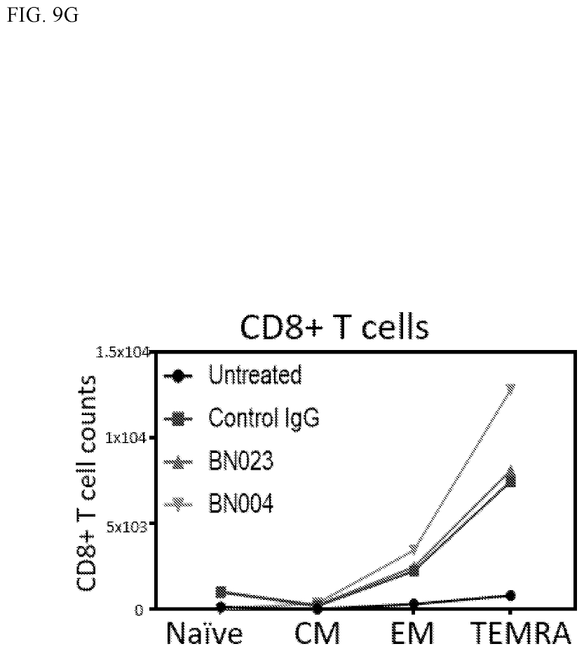

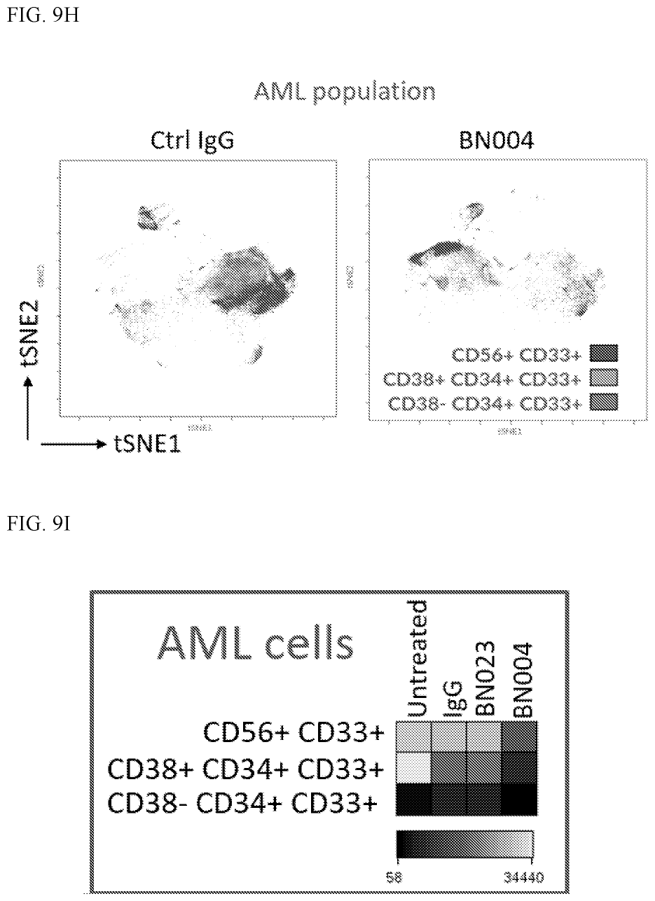

CD38 is a surface receptor which is highly expressed in hematologic malignancies cancer cells including multiple myeloma (MM), acute myelogenous leukemia (AML) and T-cell acute lymphoblastic leukemia (T-ALL). The given data show that CD38-CD3 BIONICS (BN004) has a strong killing activity against CD38+ cancer cells (MM.1S and THP-1) (FIGS. 8A-8B) after that human T cells (effector, E) were incubated overnight with CD38+ cancer cells (target, T) at the E:T ration 1:1. Lower but still significant killing activity was observed when CD38low MV4; 11 cells were used in the same experimental conditions (FIGS. 8A-8B), supporting BN004 target specificity. Cancer cell killing was indeed correlated to the ability of BN004 to activate both CD4+ and CD8+ T cells, as shown by the induction of killing (FIG. 8C) associated with the concomitant induction of early (FIG. 8D) and late (FIG. 8E) T cell activation markers when T cells were cocultured with AML cancer cells, THP-1. Cancer cell killing, and T cell activation was not observed when in the same experimental conditions cells were treated either with control IgG or with a non CD38 binding form of BIONICS mutated in a single residue (BN008) (FIGS. 8C-8E). Cancer cell killing, and T cell activation was also observed when CD38+MM (FIGS. 8F-8H) and T-ALL cells (FIGS. 81-8K) were used in the same experimental conditions. T-cell killing of cancer cells and high T cell activation was not observed when T cells were treated with BN004 and incubated with CD38 knockout HL60 AML cell lines (HL60 CD38KO) (FIGS. 8L-8M). Specific killing and significantly T cell activation was instead observed when the parental CD38+ cell line (HL60 WT) was used in the same experimental condition (FIGS. 8L-8M). Cytokine array also shows specific T cell cytokine release including IFN-γ, TNF-α and IL-2 among others in the supernatant obtained from the co-culture of T cells with CD38+ cancer cells in presence of BN004 (FIG. 8N). Cytokine release was not observed when the cells were incubated with BN023 or control IgG (FIG. 8N). Early and late CD4 and CD8 T activation (FIG. 9A) and concomitant AML killing (FIG. 9B) was also observed when BN004 was used to treat total mononuclear cells isolated from the bone marrow (BM) and peripheral blood (PB) of newly diagnosed and relapsing AML patients (FIGS. 9A-9B).

Acute myeloid leukemia (AML) accounts for approximately half of new cases of leukemia worldwide, and relapse remains a major problem. Residual leukemia stem cells (LSCs) are considered responsible to maintain AML cells. The expression of CD34 and a lack of CD38 (CD34+CD38−) on the cell surface is a pattern commonly associated with both LSCs and normal hematopoietic stem cells (HSCs); however, LSCs are considered a less primitive population. The preliminary data show that, when T cells are activated against CD38+AML blasts, they release IFN-γ, inducing the CD38-LSC population to express CD38, an effect that was not observed in normal HSCs. Because CD38 targeting is a current therapeutic option, the induction of CD38 on LSCs may provide an opportunity to specifically target LSCs while sparing normal HSCs.

Acute myeloid leukemia (AML) accounts for approximately half of new cases of leukemia worldwide, and relapse remains a major problem [1, 2]. Since 1970, AML standard treatment is cytotoxic chemotherapy. In general, for patients under 60 years, the 5-year overall survival (OS) rate after this treatment is 40-50%, while for older patients the OS rate is only 15-20% [3]. The only potentially curative treatment option currently available is allogeneic hematopoietic stem cell transplantation (allo-HSCT), which through its graft-versus-leukemia effect can eliminate residual leukemia stem cells (LSCs) [4]. Residual LSCs are considered responsible to maintain AML cells and are considered refractory to standard-of-care therapies [5]. LSCs compared to AML blasts are characterized by a more quiescent state, different metabolism, more adaptation through adhesion receptors to the bone marrow (BM), and more phenotypic plasticity [6].

Although numerous publications have shown that LSC persistence and resistance is due to the acquisition of various epigenetic, transcriptional, and metabolic characteristics, it remains unknown how to use this information to specifically therapeutically target LSCs. Clinical targeting of LSCs is still considered an unmet medical need, as this approach may increase the effectiveness of treatments and ultimately the survival of patients. Specific therapeutic targeting of LSCs without affecting normal hematopoietic stem cells (HSCs) will be crucial to achieve cures. In fact, unlike B cell malignancies that express many surface receptors exclusive to the B cell subset, such as CD19, CD20, and CD22, or BCMA in the case of multiple myeloma, the majority of the surface receptors targetable in myeloid malignancies are in common with those in normal HSCs and their myeloid and/or lymphoid progenitors [7]. For example, there are more than 20 different AML clinical trials using either CD33 or CD123 CAR T therapy or CD33/CD3 (AMG 330) or CD123/CD3 (Flotetuzumab) bispecific T-cell engagers (US National Library of Science ClinicalTrials.gov), and although these targets are ubiquitously expressed on AML blasts and LSCs, they are also highly expressed by normal HSCs and myeloid progenitors [8]. CD123-directed and CD33-directed CAR T cells and T cell engagers have both shown highly potent anti-tumor activity in pre-clinical models [9]; however, they are unable to differentiate between normal and cancerous cells. In contrast to the case of CD19 CAR T cells, where elimination of normal B cells has manageable side effects, prolonged myeloablation as a result of targeting myeloid antigens that are shared with normal myeloid progenitors may be fatal due to neutropenic infections and bleeding complications. Hence, the identification of surface receptors that can be used to specifically target both AML blasts and LSCs is considered an unmet medical need.

The expression of CD34 and a lack of CD38 (CD34+CD38−) on the cell surface is a pattern commonly associated with both LSCs and normal HSCs. However, LSCs are considered a less primitive population, as LSCs can also be found in the CD34-sub-fraction in some patients [10]. How this concept can be used to specifically target LSCs while sparing normal cells has not been clear.

Using either AML cell lines or primary cells, it was shown that CD3/TCR-mediated T cell engagement against CD38+AML blasts induces IFN-γ release. Specifically, it demonstrated that the newly developed T cell engager CD38-CD3 Biologics Nested Inside Chains (BIONIC, BN004) induces release of high levels of IFN-γ upon CD3/TCR dependent T cells engagement against CD38+AML blasts, an effect that was not found when a single non-sense mutation in the CD38 binding site was introduced (CD38 non-binding BIONIC, BN0023) or in the absence of CD38+AML target cells (FIG. 8A). The data show that CD38-CD3 BIONIC-dependent engagement of T cells to CD38+AML cells induces CD38 transcriptional activation (FIG. 8B), reduces CD34+CD38-LSCs (FIG. 8C) and significantly increases CD38 surface expression in total AML (CD45dim) population after only 48 hours of treatment (FIG. 8D). The findings reveal that, while BN004 was able to induce T cells to eliminate autologous LSCs in the BM aspirates obtained from both newly diagnosed and relapsing AML patients (FIGS. 9A-9B), this effect was not observed when a BM aspirate obtained from a healthy donor was used, as shown by colony formation assays (FIGS. 9A-9B). Moreover, BN004 was able to significantly increase survival of mice engrafted with AML cells (THP-1) (p<0.0001) and completely eradicated with no signs of relapse AML bone marrow engraftment in 33% of the mice (FIGS. 9C-9E).

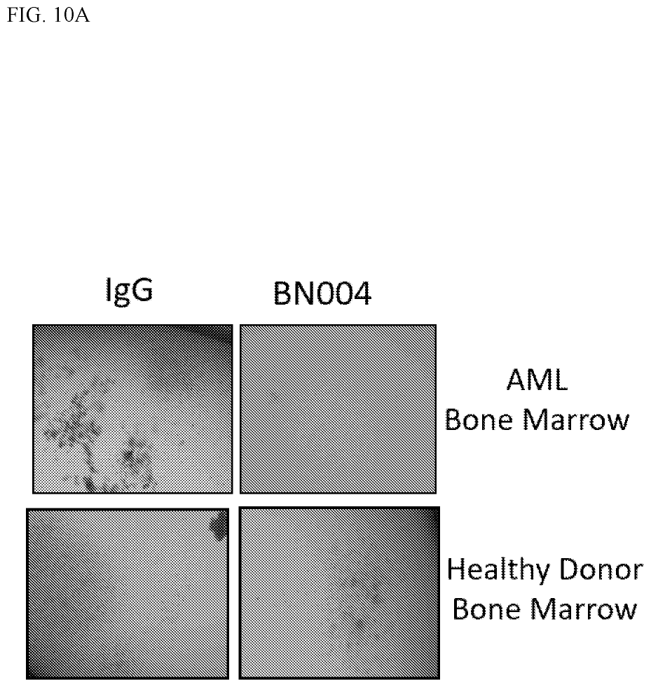

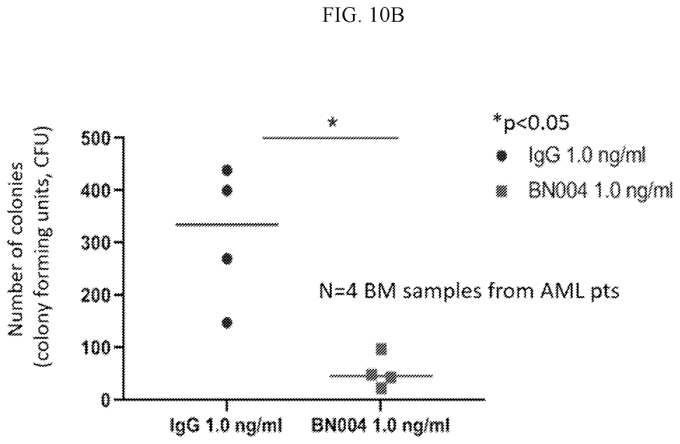

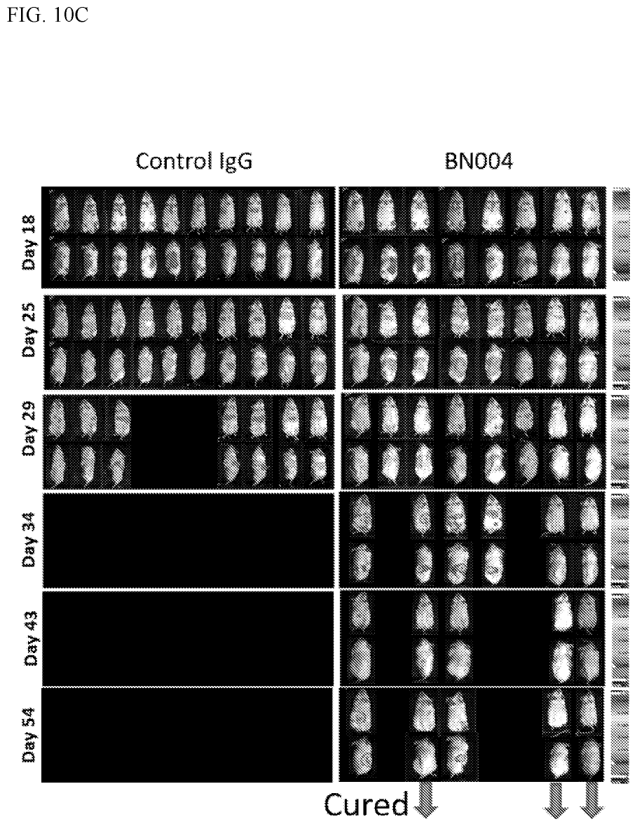

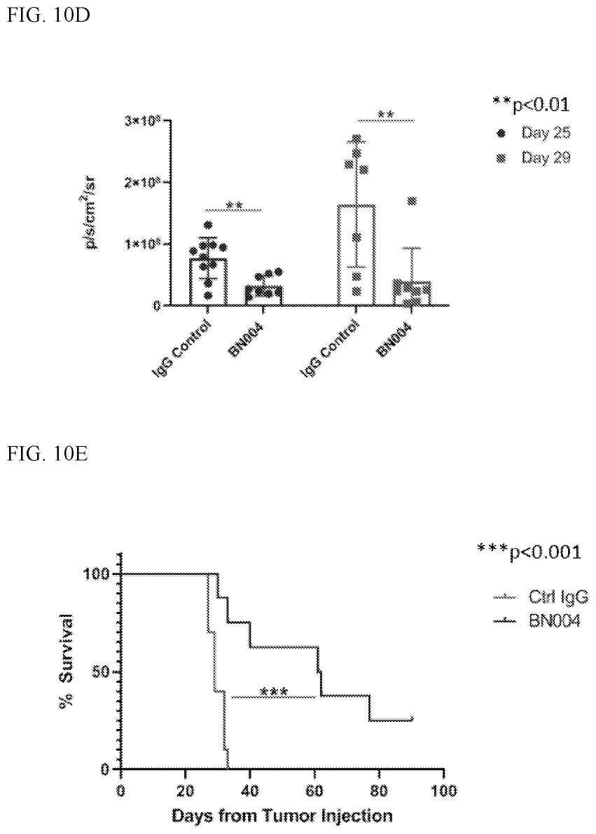

Reduction of CD34+CD38-LSCs was also observed when AML BM mononuclear cells (MNCs) or AML CD34+BM fraction were treated with IFN-γ (FIG. 10A). Conversely, decrease in CD34+CD38-HSCs was not observed when total BM MNCs and CD34+ fraction isolated from healthy donors were treated with IFN-γ in the same experimental conditions (FIG. 10B). A dose dependent increase in CD38 surface levels in total leukemia cells (CD45dim) was observed (FIG. 10C), but this effect was not found when healthy CD45dim BM-MNCs were treated (FIG. 10D). Of note IFN-γ did not affect CD34+CD38-HSCs viability. The data also show that IFN-γ treatment did not upregulate CD38 levels in total T cells (FIG. 10E) and monocytic fraction (FIGS. 10E and 10G), supporting that CD38 upregulation may be more specific of leukemia cells. Interestingly decrease in frequencies of CD38+ T cells and CD38+ monocytes were observed upon IFN-γ treatment, but total cell count was not affected, further supporting that induction of CD38 expression can be differentially regulated in non-cancer cells. While the induction of transcriptional regulation of CD38 by interferon types I and II through interferon regulatory factor-1 (IRF-1) was previously reported in leukemic B cells and in leukemia CD38+ blasts [10, 11], the exclusive CD38 re-expression on LSCs but not in healthy HSCs upon IFN-γ treatment or upon T cell engagement against AML blasts has never been reported. This observation is clinically relevant, since CD38 targeting through clinically relevant antibodies (daratumumab; isatuximab), T cell engagers (AMG424), CAR T cells or antibody-drug conjugates are current therapeutic options used in different types of cancer.

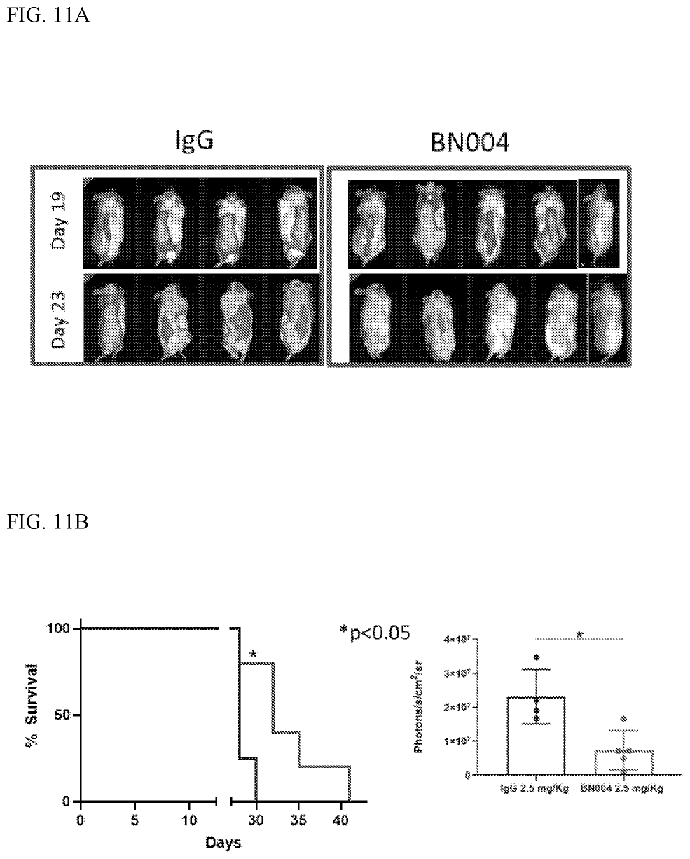

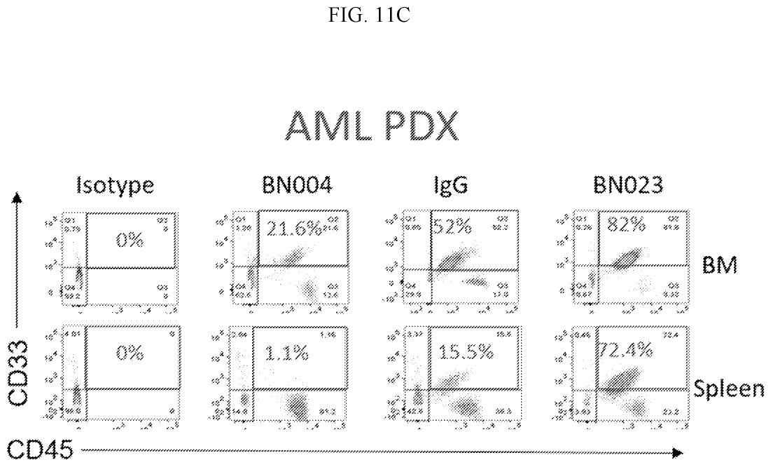

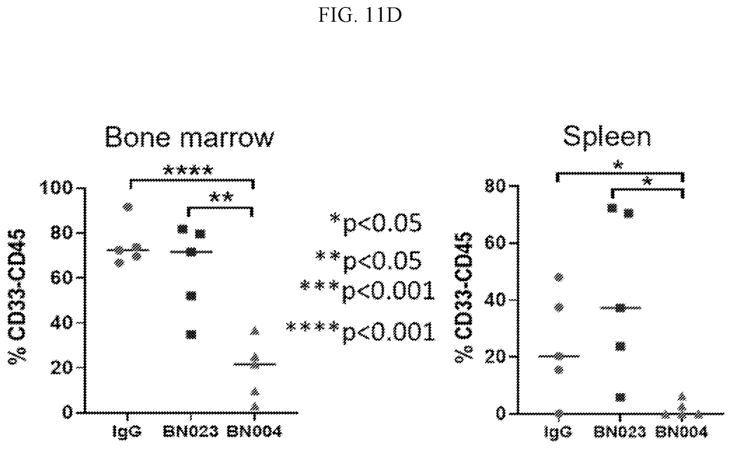

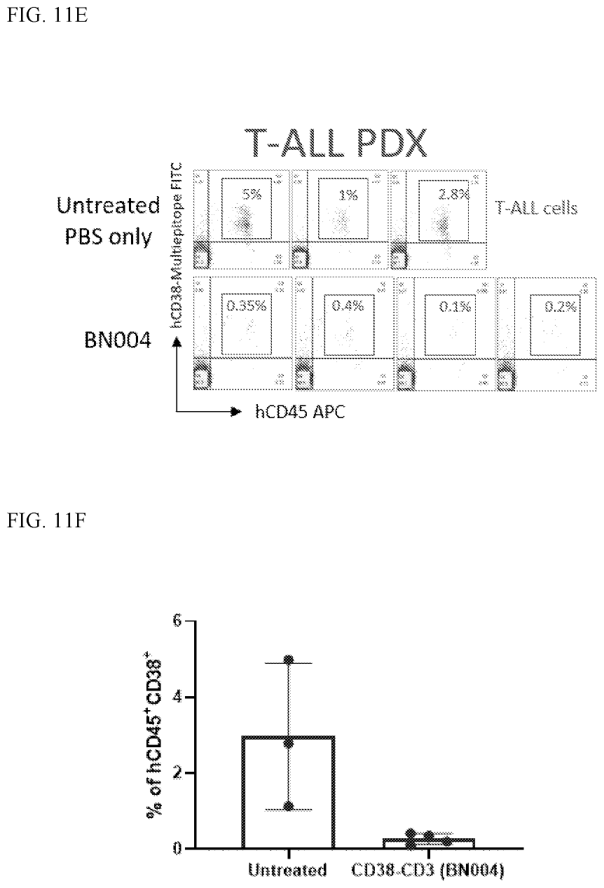

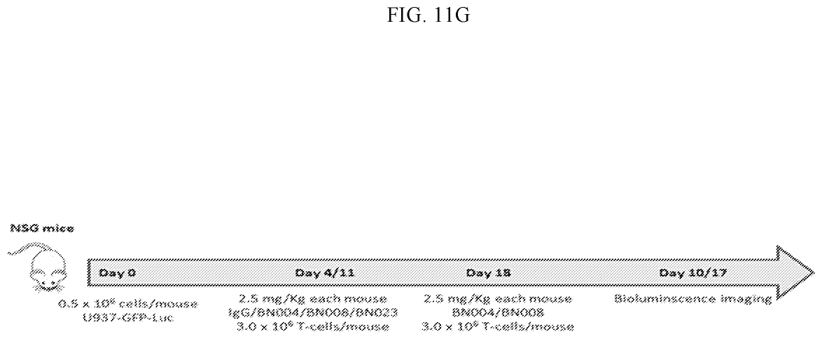

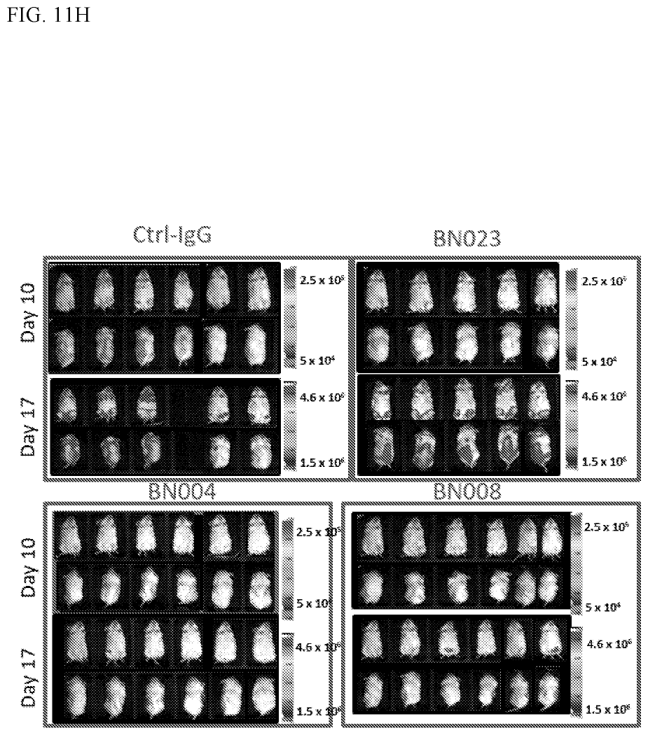

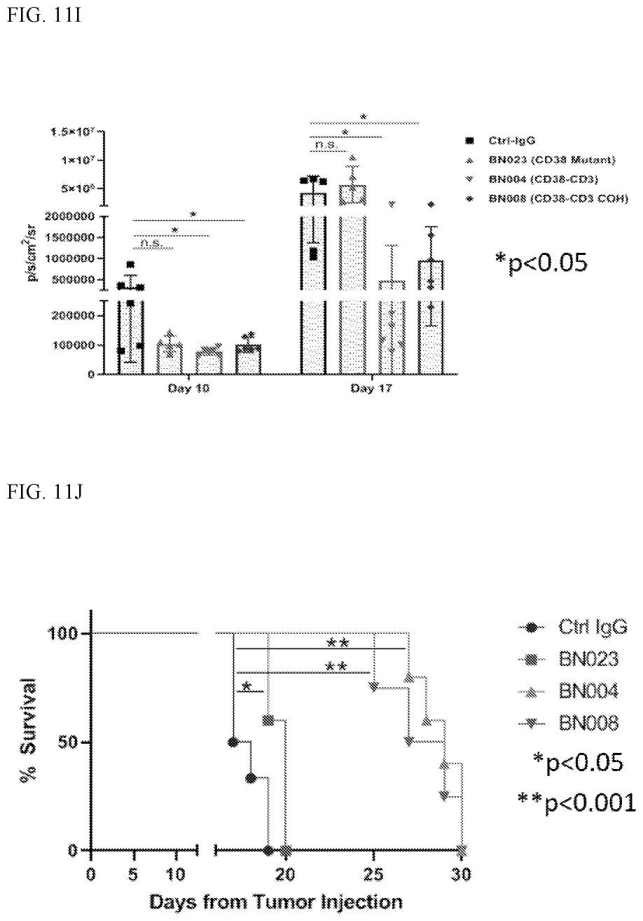

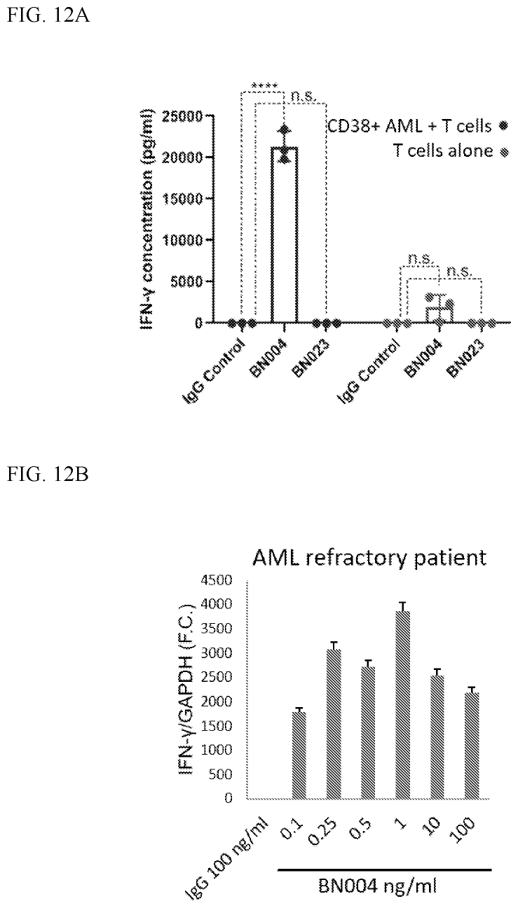

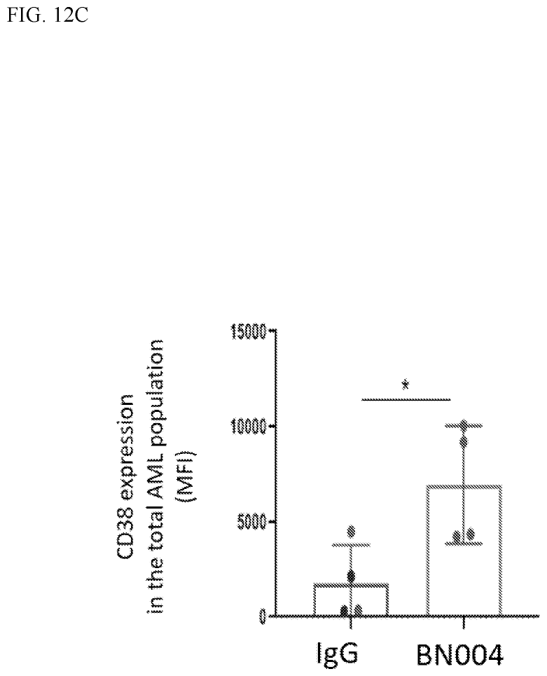

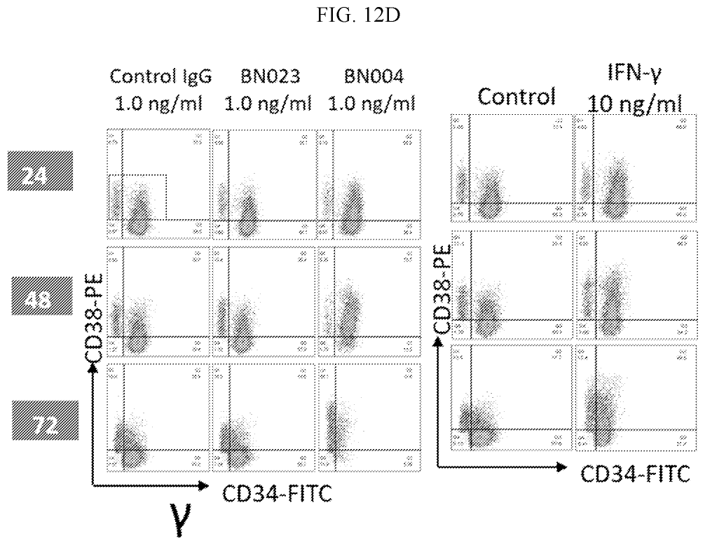

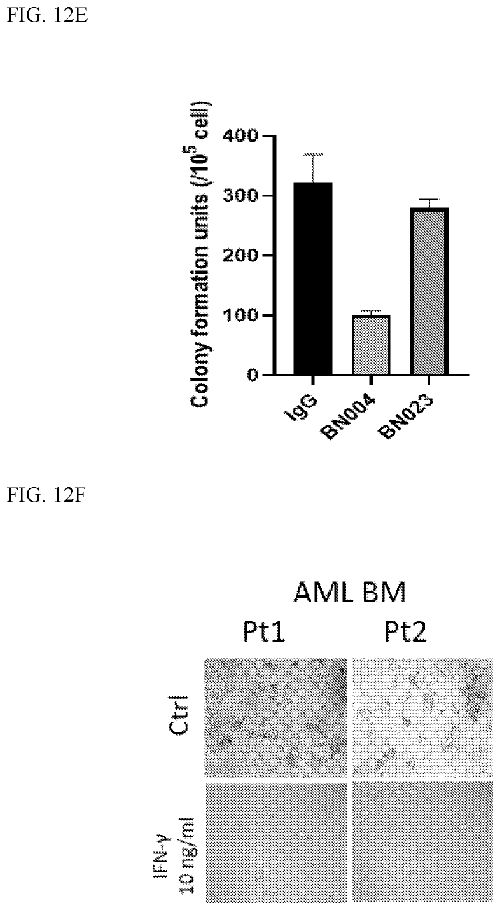

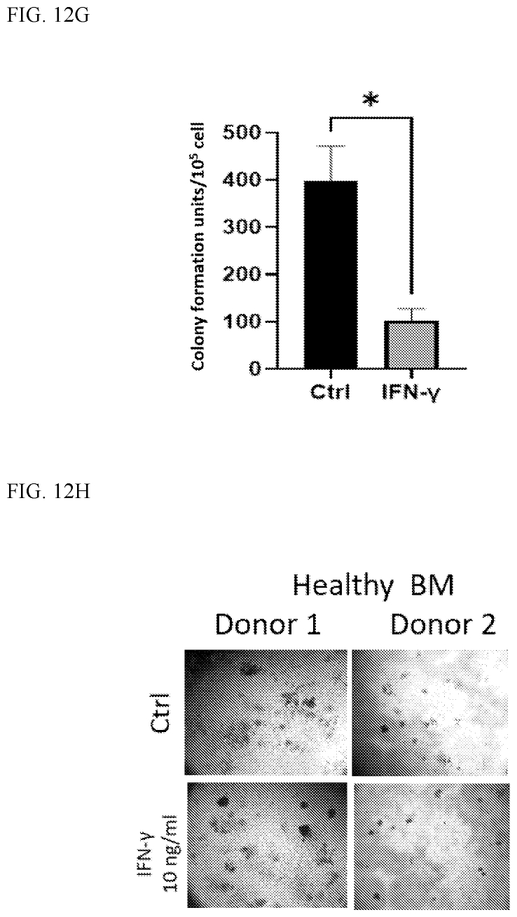

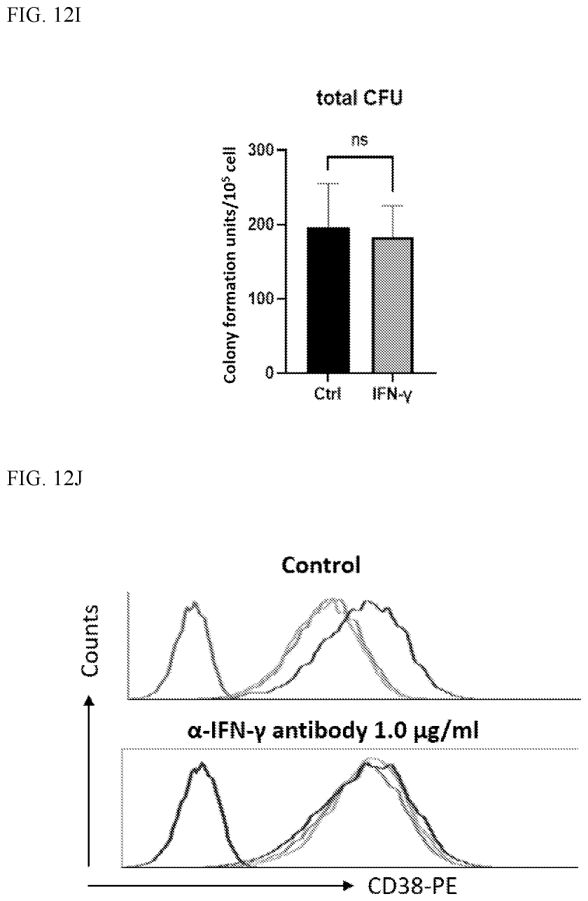

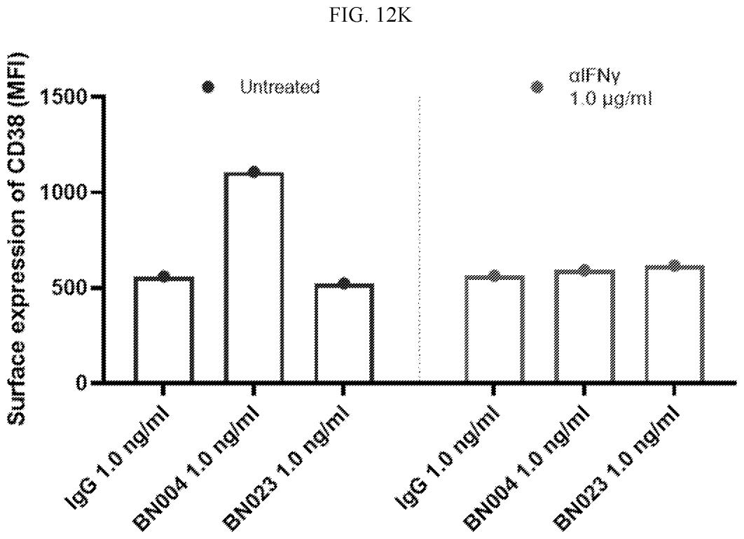

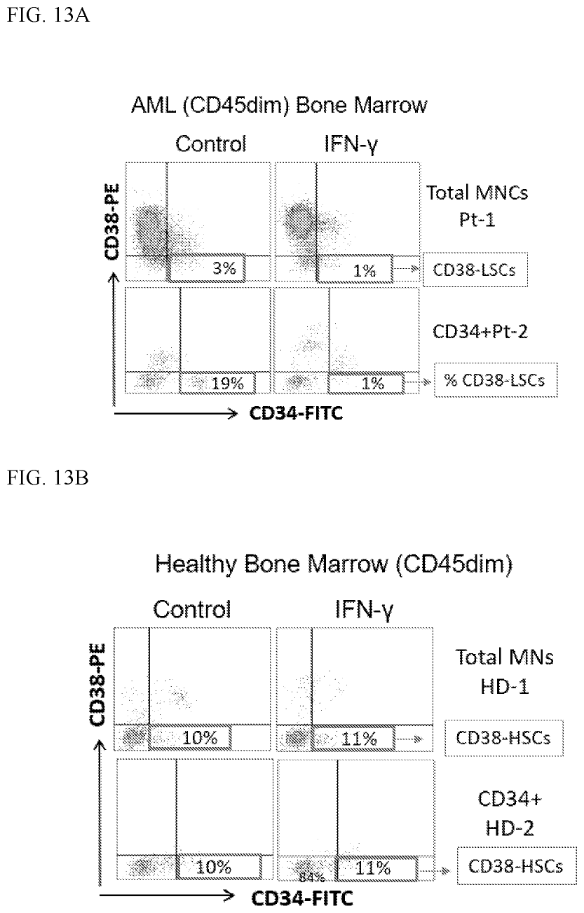

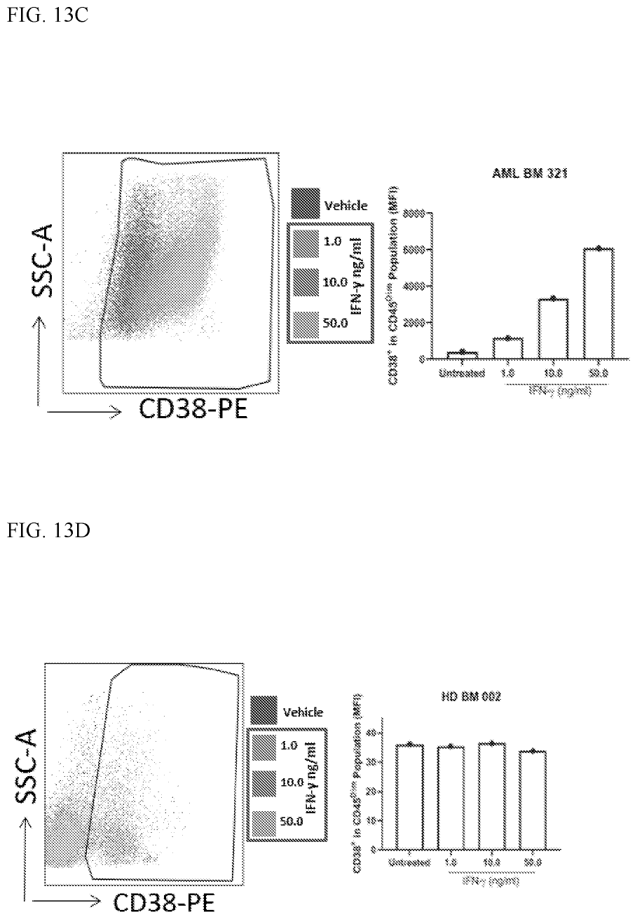

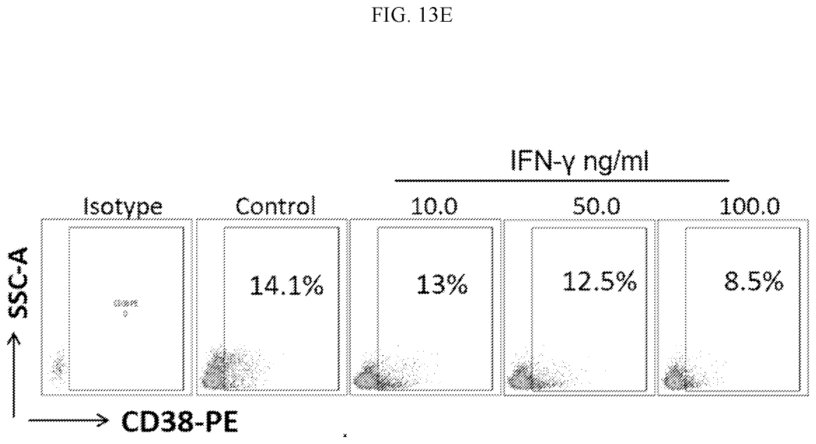

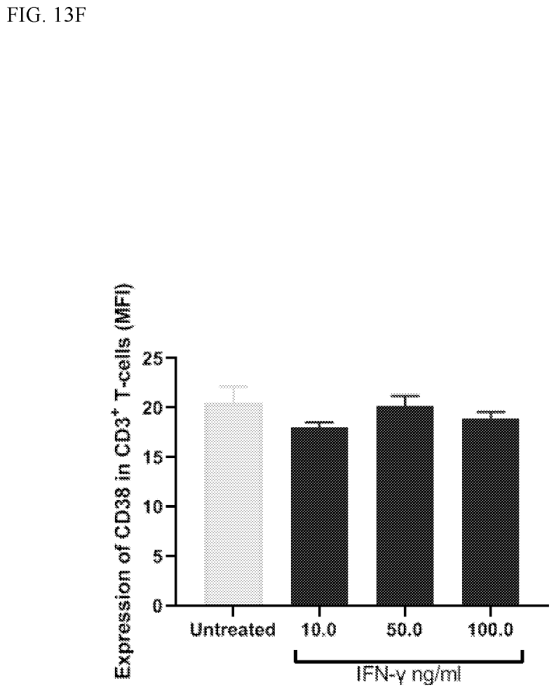

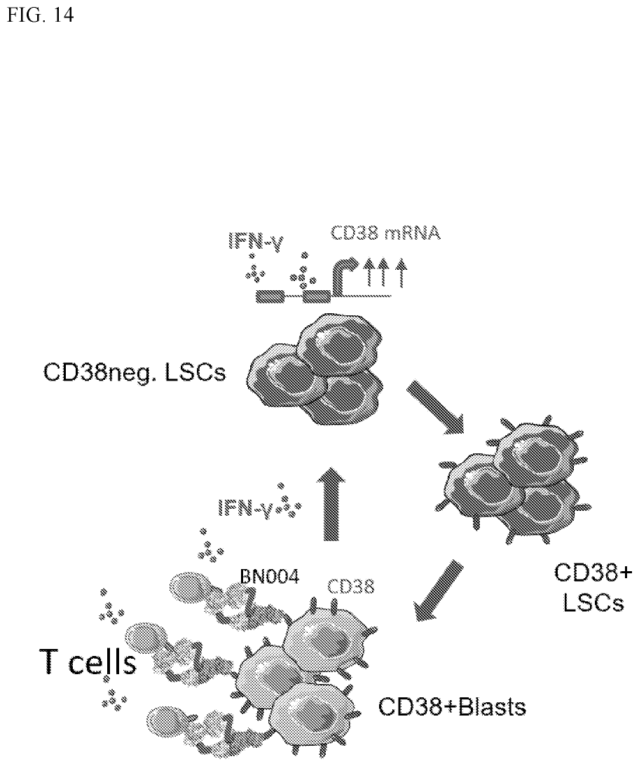

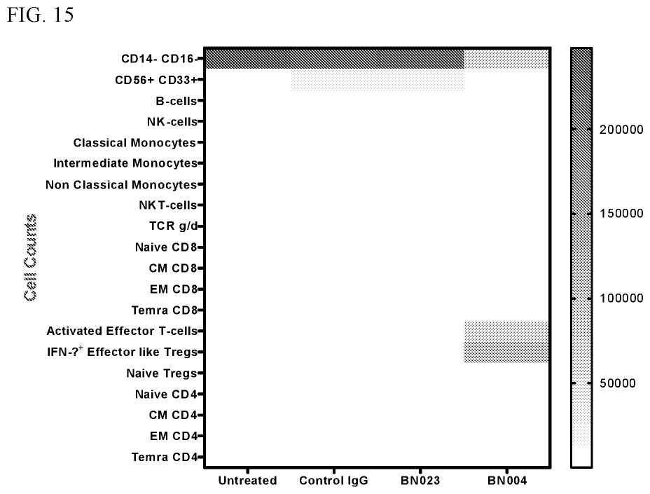

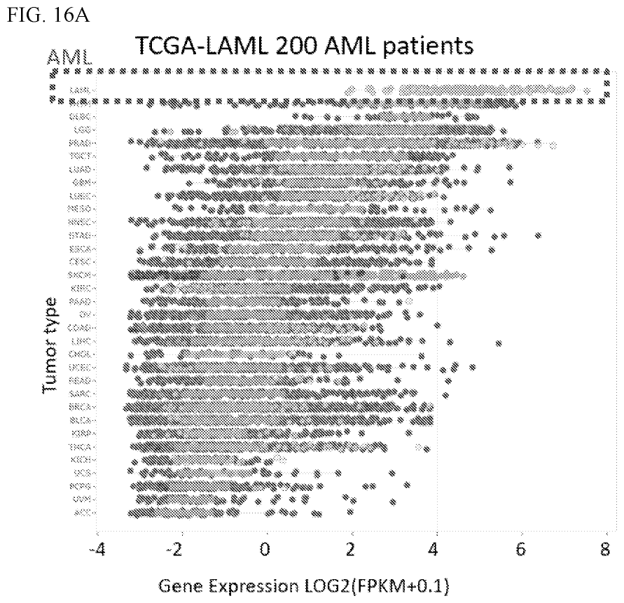

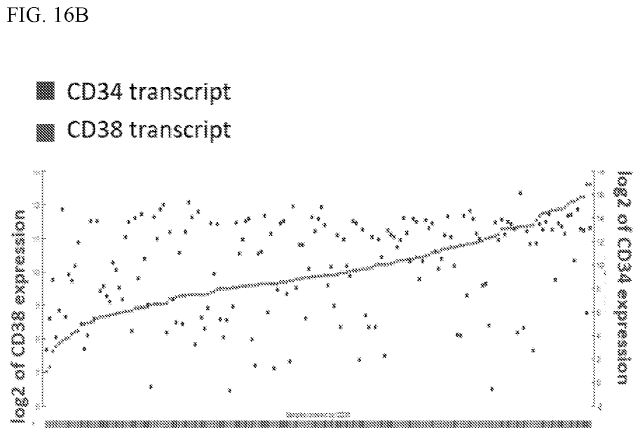

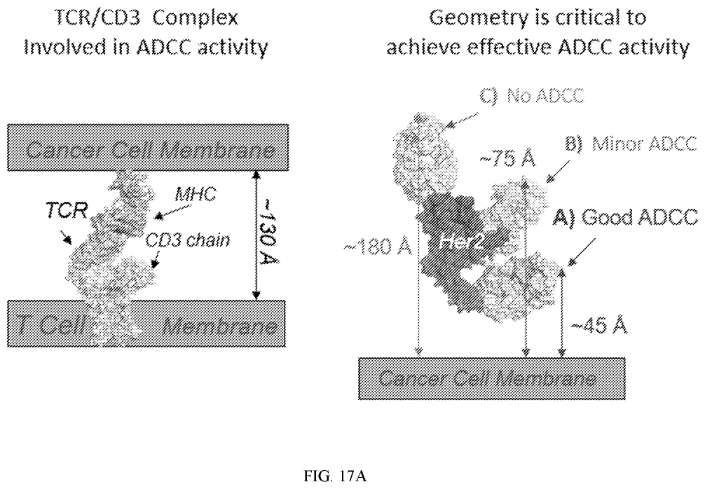

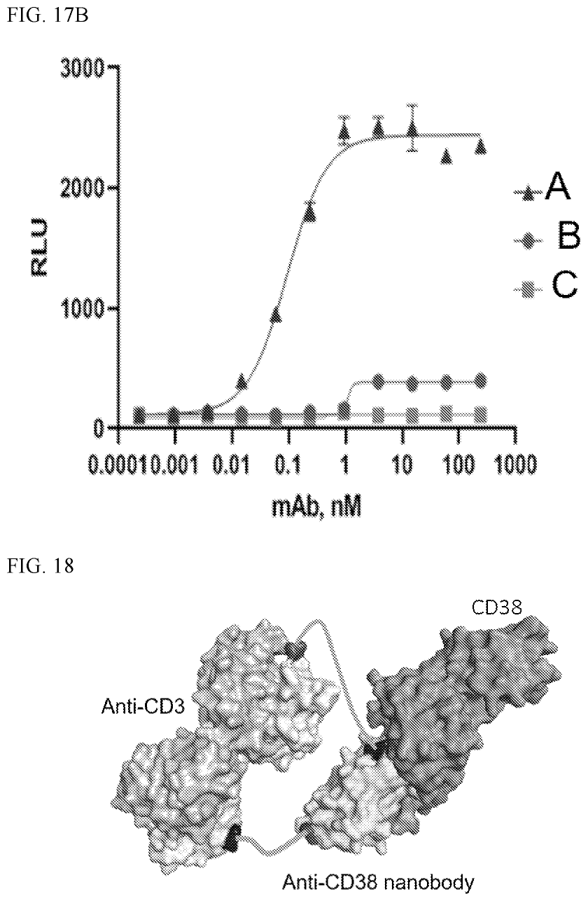

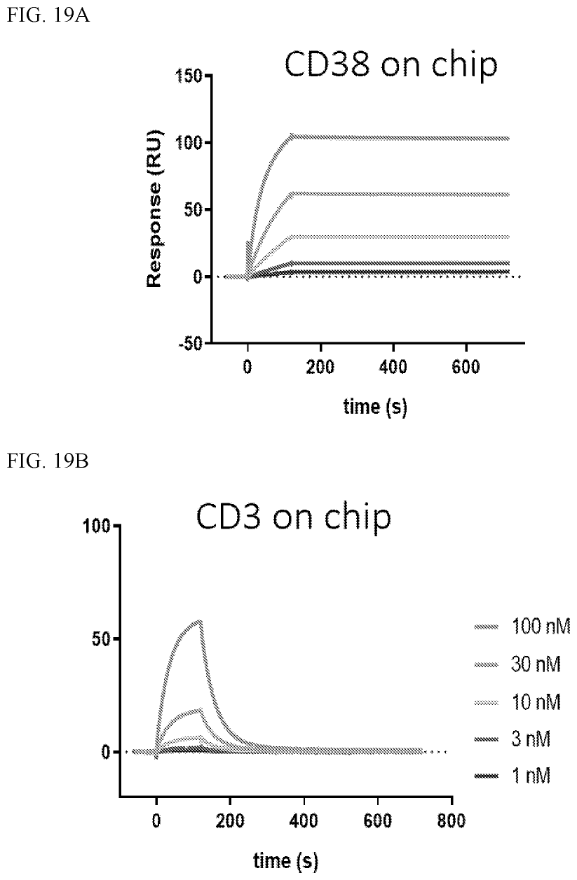

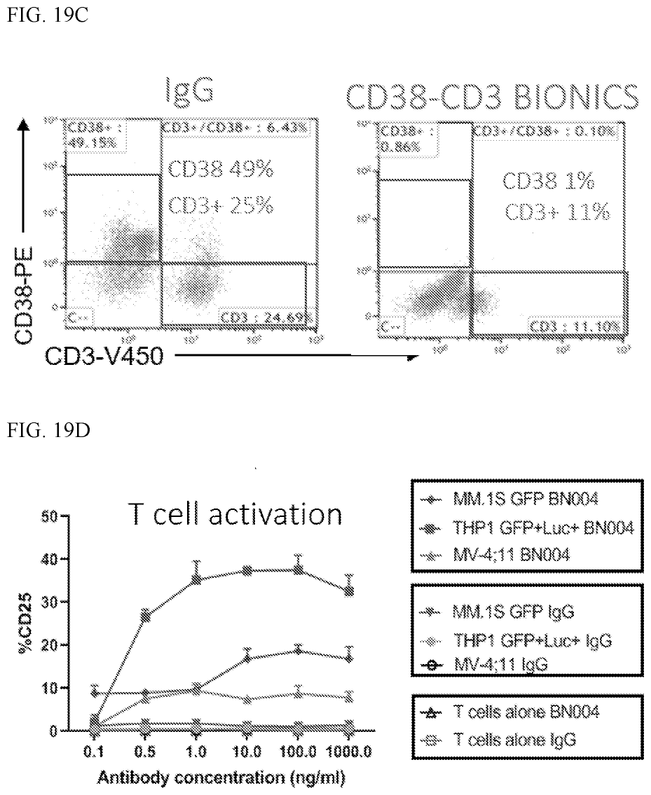

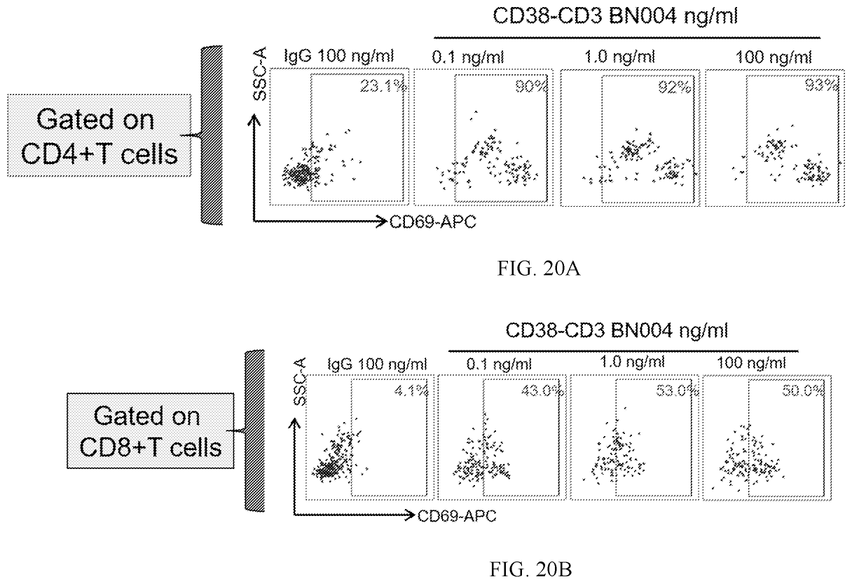

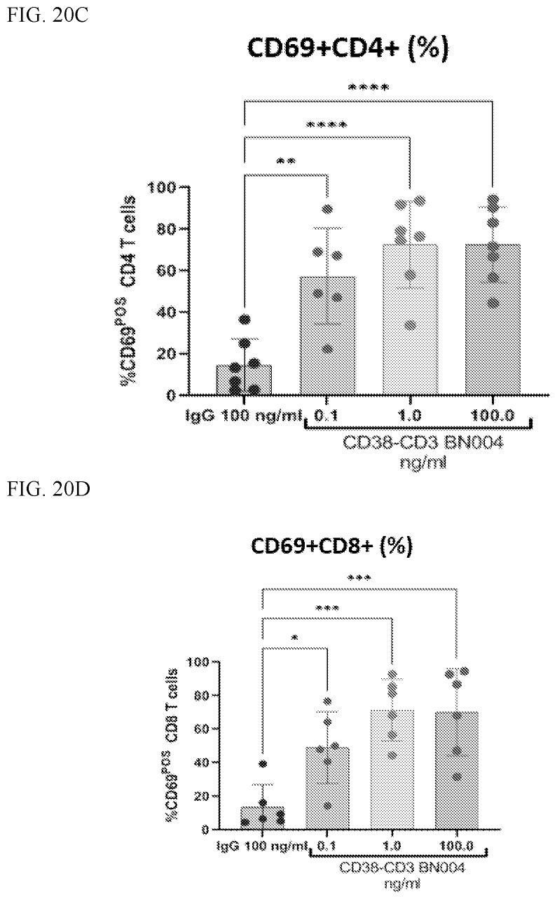

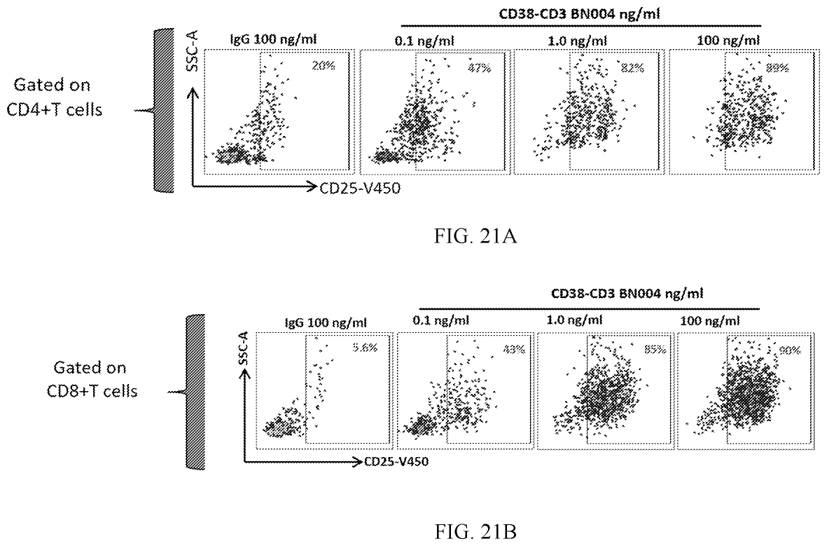

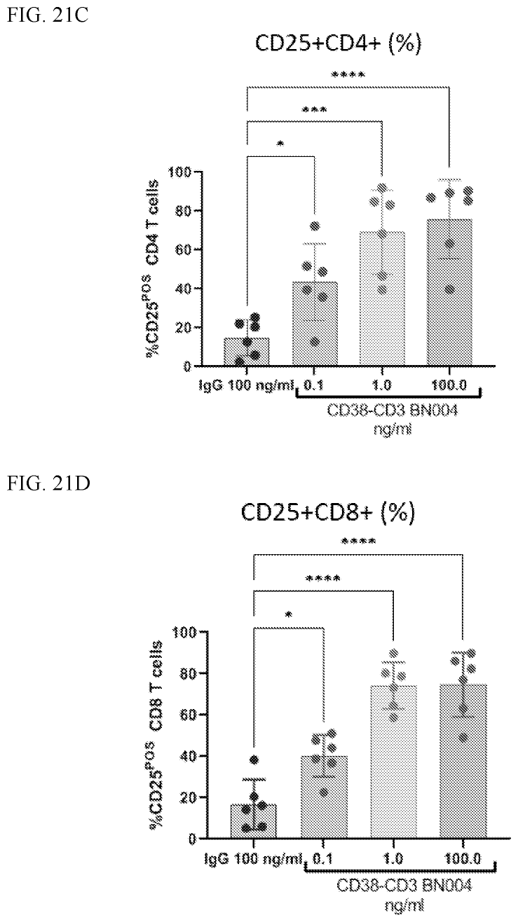

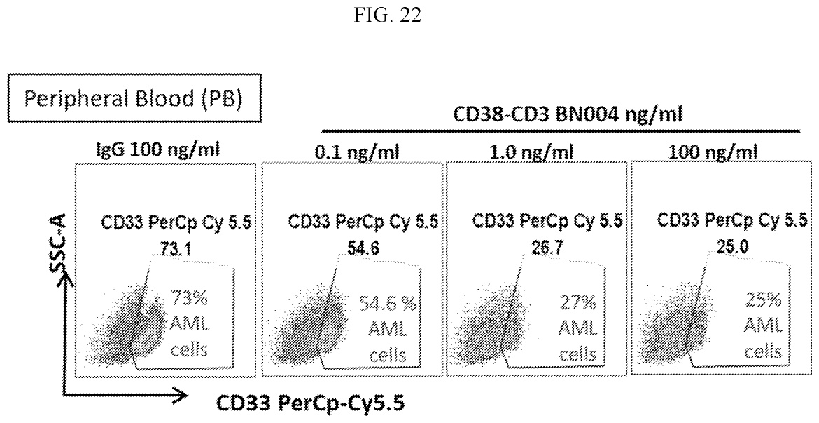

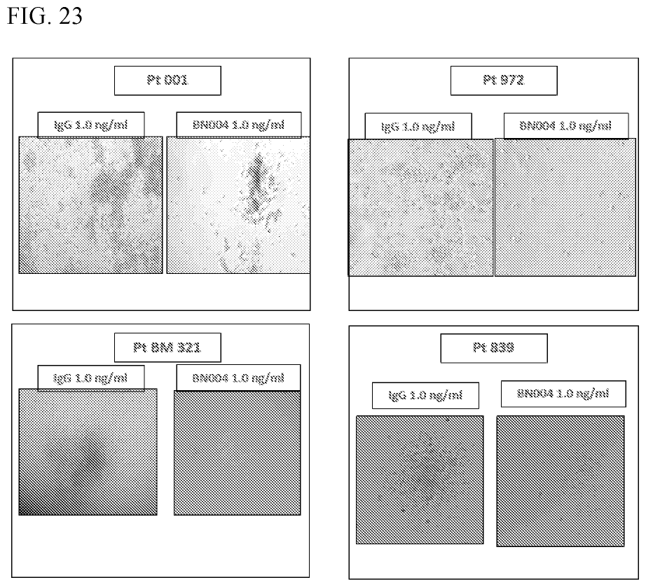

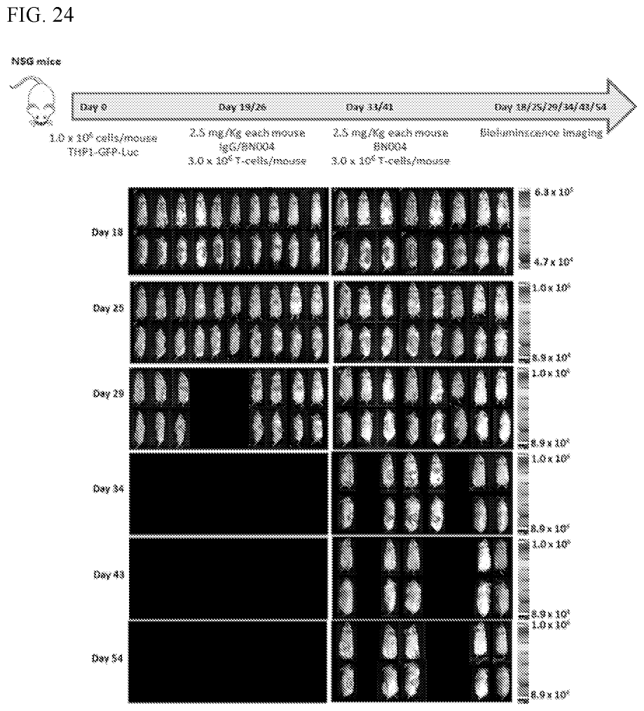

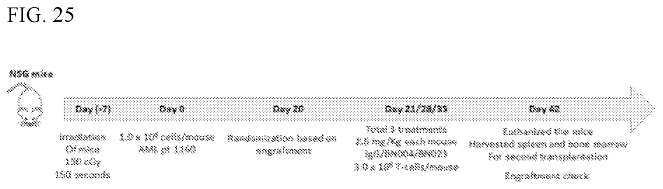

The induction of CD38 on LSCs directly through IFN-γ exposure or indirectly by targeting AML blasts (FIG. 11) provides for the first time the therapeutic opportunity to specifically target LSCs while sparing normal HSCs, an avenue toward a cure. This approach may be extremely clinically relevant, since several therapeutic interventions based on T cell engagement against AML blasts are now being introduced into the clinic, including CD33/CD123-directed bispecific antibodies and CAR-T cells (US National Library of Science ClinicalTrials.gov). The idea to combine T cell therapies against different antigens together with anti-CD38 targeted therapies, or to develop, as here, CD38-CD3 BIONIC-based T cell therapies able to induce CD38 expression and concomitantly target CD38+ blasts and LSCs, may be important therapeutic strategies toward eliminating AML.

### REFERENCES

1. Surveillance Epidemiology and End Results Program: https://seer.cancer.gov/.
2. Cancer Research UK: https://www.cancerresearchuk.org/.
3. Medeiros B C, Chan S M, Daver N G, Jonas B A, Pollyea D A: Optimizing survival outcomes with post-remission therapy in acute myeloid leukemia. Am J Hematol 2019, 94 (7): 803-811.
6. van Gils N, Denkers F, Smit L: Escape From Treatment; the Different Faces of Leukemic Stem Cells and Therapy Resistance in Acute Myeloid Leukemia. 2021, 11 (1454).
7. Cummins K D, Gill S: Will CAR T cell therapy have a role in AML? Promises and pitfalls. Seminars in hematology 2019, 56 (2): 155-163.
8. Ehninger A, Kramer M, Röllig C, Thiede C, Bornhäuser M, von Bonin M, Wermke M, Feldmann A, Bachmann M, Ehninger G et al: Distribution and levels of cell surface expression of CD33 and CD123 in acute myeloid leukemia. Blood cancer journal 2014, 4 (6): e218.
9. Gill S, Tasian S K, Ruella M, Shestova O, Li Y, Porter D L, Carroll M, Danet-Desnoyers G, Scholler J, Grupp S A et al: Preclinical targeting of human acute myeloid leukemia and myeloablation using chimeric antigen receptor-modified T cells. Blood 2014, 123 (15): 2343-2354.
10. Tanizaki R, Nomura Y, Miyata Y, Minami Y, Abe A, Hanamura A, Sawa M, Murata M, Kiyoi H, Matsushita T et al: Irrespective of CD34 expression, lineage-committed cell fraction reconstitutes and re-establishes transformed Philadelphia chromosome-positive leukemia in NOD/SCID/IL-2Rgammac−/− mice. Cancer science 2010, 101 (3): 631-638.
11. Bauvois B, Durant L, Laboureau J, Barthélémy E, Rouillard D, Boulla G, Deterre P: Upregulation of CD38 gene expression in leukemic B cells by interferon types I and II. Journal of interferon & cytokine research: the official journal of the International Society for Interferon and Cytokine Research 1999, 19 (9): 1059-1066.

### INFORMAL SEQUENCE LISTING

TABLE 1
Sequences of exemplary peptides and portions thereof.

| SEQ | | |
|---|---|---|
| ID | | |
| NO: | Description | Sequence |
| 1 | CD3 CDR L1 | RASQSIRSYLN |
| 2 | CD3 CDR L2 | AASSLQS |
| 3 | CD3 CDR L3 | QQTYSNPPIT |
| 4 | CD3 CDR H1 | GFTFDDY |
| 5 | CD3 CDR H2 | SWNSGS |
| 6 | CD3 CDR H3 | DMSGYGHYGKYGMDV |
| 7 | CD3 light | DIQMTQSPILLSASVGDRVTITCRASQSIRSYLNWYQQRTNGSPRLLIY |
| | chain | AASSLQSGVPSRFSGSGSGTDFTLTINSLQPDDEADYYCQQTYSNPPIT |
| | | FGAGTRLEIKRTVAAPSVFIFPPSDEQLKSGTASVVCLLNNFYPREAKV |
| | | QWKVDNALQSGNSQESVTEQDSKDSTYSLSSTLTLSKADYEKHKVYA |
| | | CEVTHQGLSSPVTKSFNRGEC |
| 8 | CD3 heavy | EVQLVESGGGLVQPGRSLRLSCAASGFTFDDYTMHWVRQAPGKGLE |
| | chain | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSC |
| 9 | Linker L1 | AASGGAGS |
| 10 | Linker L2 | GAVPLSLYGAGG |
| 11 | Linker L2 | SSGASASAAGGS |
| 12 | CD38 CDR L1 | GRTFRNY |
| | (5F1K) | |
| 13 | CD38 CDR L2 | TWVGAS |
| | (5F1K) | |
| 14 | CD38 CDR L3 | GRGIVAGRIPAEYAD |
| | (5F1K) | |
| 15 | CD38 | DVQLQESGGGLVQAGGSLRLSCTGSGRTFRNYPMAWFRQAPGKER |
| | nanobody | EFVAGITWVGASTLYADFAKGRFTISRDNAKNTVYLQMNSLKPEDTA |
| | (5F1K) | VYSCAAGRGIVAGRIPAEYADWGQGTQVTV |
| 16 | 3-38BN001 | EVQLVESGGGLVQPGRSLRLSCAASGFTFDDYTMHWVRQAPGKGLE |
| | (5F1K) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGLVQAGGSLRLSCTGSGRTFRNYPMAWFRQAPGKE |
| | | REFVAGITWVGASTLYADFAKGRFTISRDNAKNTVYLQMNSLKPEDT |
| | | AVYSCAAGRGIVAGRIPAEYADWGQGTQVTVSSGAVPLSLYGAGGDI |
| | | QMTQSPILLSASVGDRVTITCRASQSIRSYLNWYQQRTNGSPRLLIYA |
| | | ASSLQSGVPSRFSGSGSGTDFTLTINSLQPDDEADYYCQQTYSNPPITF |
| | | GAGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| 17 | 3-38BN002 | EVQLVESGGGLVQPGRSLRLSCAASGFTFDDYTMHWVRQAPGKGLE |
| | (5F10) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGLVQAGHSLRLSCVGSGSRFDNYAMGWFRQAPGKE |
| | | REFVAAISWSSGTTRYLDTVKGRFTISRDNAKSTVYLQMNSLKPEDTA |
| | | VYYCAARYQPRYYDSGDMDGYEYDNWGQGTQVTVSSGAVPLSLYG |
| | | AGGDIQMTQSPILLSASVGDRVTITCRASQSIRSYLNWYQQRTNGSP |
| | | RLLIYAASSLQSGVPSRFSGSGSGTDFTLTINSLQPDDEADYYCQQTYS |
| | | NPPITFGAGTRLEIKRTVAAPSVFIFPPSDEQLKSGTASVVCLLNNFYPR |
| | | EAKVQWKVDNALQSGNSQESVTEQDSKDSTYSLSSTLTLSKADYEKH |
| | | KVYACEVTHQGLSSPVTKSFNRGEC |
| 18 | 3-38BN003 | EVQLVESGGGLVQPGRSLRLSCAASGFTFDDYTMHWVRQAPGKGLE |
| | (5F21) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGSVQAGGSLTLSCTASGLLFRLASMGWYRQAPGKER |
| | | ELIATITVGGKTNYKDSVQGRFIITRDNTGDNTKSTVTLQMNRLKPED |
| | | TAVYYCNTASPAVGADTWGQGTRVTVSSGAVPLSLYGAGGDIQMT |
| | | QSPILLSASVGDRVTITCRASQSIRSYLNWYQQRTNGSPRLLIYAASSL |
| | | QSGVPSRFSGSGSGTDFTLTINSLQPDDEADYYCQQTYSNPPITFGAG |
| | | TRLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTH |
| | | QGLSSPVTKSFNRGEC |
| 19 | 3-38BN004 | EVQLVESGGGLVQPGRSLRLSCAASGFTFDDYTMHWVRQAPGKGLE |
| | (5F1K) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGLVQAGGSLRLSCTGSGRTFRNYPMAWFRQAPGKE |
| | | REFVAGITWVGASTLYADFAKGRFTISRDNAKNTVYLQMNSLKPEDT |
| | | AVYSCAAGRGIVAGRIPAEYADWGQGTQVTVSSSSGASASAAGGSDI |
| | | QMTQSPILLSASVGDRVTITCRASQSIRSYLNWYQQRTNGSPRLLIYA |
| | | ASSLQSGVPSRFSGSGSGTDFTLTINSLQPDDEADYYCQQTYSNPPITF |
| | | GAGTRLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYAC |
| | | EVTHQGLSSPVTKSFNRGEC |
| 20 | 3-38BN005 | EVQLVESGGGLVQPGRSLRLSCAASGFTFDDYTMHWVRQAPGKGLE |
| | (5F10) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGLVQAGHSLRLSCVGSGSRFDNYAMGWFRQAPGKE |
| | | REFVAAISWSSGTTRYLDTVKGRFTISRDNAKSTVYLQMNSLKPEDTA |
| | | VYYCAARYQPRYYDSGDMDGYEYDNWGQGTQVTVSSSSGASASAA |
| | | GGSDIQMTQSPILLSASVGDRVTITCRASQSIRSYLNWYQQRTNGSPR |
| | | LLIYAASSLQSGVPSRFSGSGSGTDFTLTINSLQPDDEADYYCQQTYSN |
| | | PPITFGAGTRLEIKRTVAAPSVFIFPPSDEQLKSGTASVVCLLNNFYPRE |
| | | AKVQWKVDNALQSGNSQESVTEQDSKDSTYSLSSTLTLSKADYEKHK |
| | | VYACEVTHQGLSSPVTKSFNRGEC |
| 21 | 3-38BN006 | EVQLVESGGGLVQPGRSLRLSCAASGFTFDDYTMHWVRQAPGKGLE |
| | (5F21) | WVSDISWNSGSIGYADSVKGRFTVSRDNAKNSLYLQMNSLRGEDTAI |
| | | YYCAKDMSGYGHYGKYGMDVWGQGTTVTVSSASTKGPSVFPLAPS |
| | | SKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCAASGGAG |
| | | SDVQLQESGGGSVQAGGSLTLSCTASGLLFRLASMGWYRQAPGKER |
| | | ELIATITVGGKTNYKDSVQGRFIITRDNTGDNTKSTVTLQMNRLKPED |
| | | TAVYYCNTASPAVGADTWGQGTRVTVSSSSGASASAAGGSDIQMT |
| | | QSPILLSASVGDRVTITCRASQSIRSYLNWYQQRTNGSPRLLIYAASSL |
| | | QSGVPSRFSGSGSGTDFTLTINSLQPDDEADYYCQQTYSNPPITFGAG |
| | | TRLEIKRTVAAPSVFIFPPSDEQLKSGTASVVCLLNNFYPREAKVQWKV |
| | | DNALQSGNSQESVTEQDSKDSTYSLSSTLTLSKADYEKHKVYACEVTH |
| | | QGLSSPVTKSFNRGEC |
| 22 | CD38 CDR L1 | SRFDNY |
| | (5F10) | |
| 23 | CD38 CDR L2 | WSSGT |
| | (5F10) | |
| 24 | CD38 CDR L3 | YQPRYYDSGDMDGYEYDN |
| | (5F10) | |
| 25 | CD38 | DVQLQESGGGLVQAGHSLRLSCVGSGSRFDNYAMGWFRQAPGKER |
| | nanobody | EFVAAISWSSGTTRYLDTVKGRFTISRDNAKSTVYLQMNSLKPEDTAV |
| | (5F10) | YYCAARYQPRYYDSGDMDGYEYDNWGQGTQVTV |
| 26 | CD38 CDR L1 | LLFRLAS |
| | (5F21) | |
| 27 | CD38 CDR L2 | NTGDNT |
| | (5F21) | |
| 28 | CD38 CDR L3 | SPAVGADT |
| | (5F21) | |
| 29 | CD38 | DVQLQESGGGSVQAGGSLTLSCTASGLLFRLASMGWYRQAPGKERE |
| | nanobody | LIATITVGGKTNYKDSVQGRFIITRDNTGDNTKSTVTLQMNRLKPEDT |
| | (5F21) | AVYYCNTASPAVGADTWGQGTRVTV |
| 30 | Linker L2 | SSSSGASASAAGGS |
| 31 | Linker L2 | SSGAVPLSLYGAGG |
| SEQ ID | (26H8) CD3 | KSSQSLLNSRTRKNYLA |
| NO: 32 | CDR L1 | |
| SEQ ID | (26H8) CD3 | WASTRES |
| NO: 33 | CDR L2 | |
| SEQ ID | (26H8) CD3 | KQSYILRT |
| NO: 34 | CDR L3 | |
| SEQ ID | (26H8) CD3 | GYTFTNY |
| NO: 35 | CDR H1 | |
| SEQ ID | (26H8) CD3 | YCGDGS |
| NO: 36 | CDR H2 | |
| SEQ ID | (26H8) CD3 | DWAGSYFFDF |
| NO: 37 | CDR H3 | |
| SEQ ID | (humanized | DIQMTQSPSSLSASVGDRVTITCKSSQSLLNSRTRKNYLAWYQQKPGKAP |
| NO: 38 | 26H8) CD3 | KLLIYWASTRESGVPSRFSGSGSGTDFTFTISSLQPEDIATYYCKQSYIL |
| | variable VL | RTFGGGTKVEIK |
| SEQ ID | (humanized | QVQLQESGPGLVKPSETLSLTCTASGYTFTNYYIHWVRQSPGKGLEWIGW |
| NO: 39 | 26H8) CD3 | IYCGDGSTKFNEQFKDRTTLSADHSKTQASLKLSSVTAADTAIYFCARDW |
| | variable VH | AGSYFFDFWGQGTMVTVSS |
| SEQ ID | (26H8) CD3 | DIVMSQSPSSLAVSAGEKVTMSCKSSQSLLNSRTRKNYLAWYQQKPGQSP |
| NO: 40 | variable VL | KLLIYWASTRESGVPDRFTGSGSGTDFTLTISSVQAEDLAVYYCKQSYIL |
| | | RTFGGGTKLEIK |
| SEQ ID | (26H8) CD3 | QVQLQQSGPELVKPGASVRMSCKASGYTFTNYYIHWVRQRPGQGLEWIGW |
| NO: 41 | variable VH | IYCGDGSTKFNEQFKDRTTLTADHSSSTAYMLLSSLTSEDSAIYFCARDW |
| | | AGSYFFDFWGQGTTLTVSS |
| SEQ ID | (3F11) CD3 | DIVMSQSPSSLAVSAGEKVTISCKSSQSLLNNRTRKNYLAWYQQKPGQSP |
| NO: 42 | variable VL | KLLIYWASTRESGVPDRFTGSGSGTDFTLTISSVQAEDLAVYYCKQSYIL |
| | | RTFGGGTKLEIK |
| SEQ ID | (3F11) CD3 | QVQLQQSGPDLVRPGASVKMSCKASGYTFTNYYVHWLNQRPGQGLEWIGW |
| NO: 43 | variable VH | IYPGDGSTKFNEKFKGKTTLTADKSSSTAYILLSSLTSADSAIYFCTRDA |
| | | GYYFDVWGAGTTVTVSS |

### EMBODIMENTS

Embodiment 1. A peptide comprising: (i) a first anti-CD3 dimerizing domain bound to a CD38 binding domain through a first chemical linker; and (ii) a second anti-CD3 dimerizing domain bound to said CD38 binding domain through a second chemical linker; wherein said first anti-CD3 dimerizing domain is capable of non-covalently binding to said second anti-CD3 domain to form an anti-CD3 binding domain.

Embodiment 2. The peptide of embodiment 1, wherein said peptide further comprises a covalent bond connecting said first anti-CD3 dimerizing domain and said second anti-CD3 dimerizing domain.

Embodiment 3. The peptide of embodiment 1 or 2, wherein said first anti-CD3 dimerizing domain is bound to said second anti-CD3 dimerizing domain.

Embodiment 4. The peptide of any one of embodiments 1-3, wherein said first chemical linker is bound to the N-terminus of said CD38 binding domain and said second chemical linker is bound to the C-terminus of said CD38 binding domain.

Embodiment 5. The peptide of any one of embodiments 1-3, wherein said first chemical linker is bound to the C-terminus of said CD38 binding domain and said second chemical linker is bound to the N-terminus of said CD38 binding domain.

Embodiment 6. The peptide of any one of embodiments 1-5, wherein said first anti-CD3 dimerizing domain comprises a variable light chain domain.

Embodiment 7. The peptide of any one of embodiments 1-6, wherein said first anti-CD3 dimerizing domain comprises a constant light chain domain.

Embodiment 8. The peptide of embodiment 7, wherein said variable light chain domain is bound to said CD38 binding domain through said constant light chain domain.

Embodiment 9. The peptide of any one of embodiments 1-8, wherein said first anti-CD3 dimerizing domain is an antibody light chain.

Embodiment 10. The peptide of any one of embodiments 1-9, wherein said second anti-CD3 dimerizing domain comprises a variable heavy chain domain.

Embodiment 11. The peptide of any one of embodiments 1-10, wherein said second anti-CD3 dimerizing domain comprises a constant heavy chain domain.

Embodiment 12. The peptide of embodiment 11, wherein said constant heavy chain domain is bound to said CD38 binding domain through said variable heavy chain domain.

Embodiment 13. The peptide of any one of embodiments 1-12, wherein said second anti-CD3 dimerizing domain is an antibody heavy chain.

Embodiment 14. The peptide of any one of embodiments 1-5, wherein said first anti-CD3 dimerizing domain comprises a variable heavy chain domain.

Embodiment 15. The peptide of embodiment 14, wherein said first anti-CD3 dimerizing domain comprises a constant heavy chain domain.

Embodiment 16. The peptide of embodiment 15, wherein said variable heavy chain domain is bound to said CD38 binding domain through said constant heavy chain domain.

Embodiment 17. The peptide of any one of embodiments 14-16, wherein said first anti-CD3 dimerizing domain is an antibody heavy chain.

Embodiment 18. The peptide of any one of embodiments 1-5 or 14-17, wherein said second anti-CD3 dimerizing domain comprises a variable light chain domain.

Embodiment 19. The peptide of embodiment 18, wherein said second anti-CD3 dimerizing domain comprises a constant light chain domain.

Embodiment 20. The peptide of embodiment 19, wherein said constant light chain domain is bound to said CD38 binding domain through said variable light chain domain.

Embodiment 21. The peptide of any one of embodiments 1-5 or 14-20, wherein said second anti-CD3 dimerizing domain is an antibody light chain.

Embodiment 22. The peptide of any one of embodiments 1-21, wherein said anti-CD3 binding domain is a Fab domain.

Embodiment 23. The peptide of any one of embodiments 1-22, wherein said first anti-CD3 dimerizing domain is an antibody light chain comprising a CDR L1 as set forth in SEQ ID NO: 1, a CDR L2 as set forth in SEQ ID NO:2 and a CDR L3 as set forth in SEQ ID NO:3.

Embodiment 24. The peptide of embodiment 23, wherein said antibody light chain comprises the sequence of SEQ ID NO:7.

Embodiment 25. The peptide of any one of embodiments 1-22, wherein said first anti-CD3 dimerizing domain is an antibody heavy chain comprising a CDR H1 as set forth in SEQ ID NO:4, a CDR H2 as set forth in SEQ ID NO:5, and a CDR H3 as set forth in SEQ ID NO: 6.

Embodiment 26. The peptide of embodiment 25, wherein said antibody heavy chain comprises the sequence of SEQ ID NO:8.

Embodiment 27. The peptide of any one of embodiments 1-24, wherein said second anti-CD3 dimerizing domain is an antibody heavy chain comprising a CDR H1 as set forth in SEQ ID NO:4, a CDR H2 as set forth in SEQ ID NO:5, and a CDR H3 as set forth in SEQ ID NO: 6.

Embodiment 28. The peptide of embodiment 27, wherein said antibody heavy chain comprises the sequence of SEQ ID NO:8.

Embodiment 29. The peptide of any one of embodiments 1-22 or 25-26, wherein said second anti-CD3 dimerizing domain is an antibody light chain comprising a CDR L1 as set forth in SEQ ID NO:1, a CDR L2 as set forth in SEQ ID NO:2 and a CDR L3 as set forth in SEQ ID NO: 3.

Embodiment 30. The peptide of any one of embodiments 1-22, wherein said first anti-CD3 dimerizing domain is an antibody light chain comprising a CDR L1 as set forth in SEQ ID NO: 32, a CDR L2 as set forth in SEQ ID NO:33 and a CDR L3 as set forth in SEQ ID NO: 34.

Embodiment 31. The peptide of any one of embodiments 1-22 or 30, wherein said second anti-CD3 dimerizing domain is an antibody heavy chain comprising a CDR H1 as set forth in SEQ ID NO:35, a CDR H2 as set forth in SEQ ID NO:36 and a CDR H3 as set forth in SEQ ID NO:37.

Embodiment 32. The peptide of embodiment 30, wherein said antibody light chain comprises the sequence of SEQ ID NO:38, SEQ ID NO:40 or SEQ ID NO:42.

Embodiment 33. The peptide of embodiment 31, wherein said antibody heavy chain comprises the sequence of SEQ ID NO:39, SEQ ID NO:41 or SEQ ID NO:43.

Embodiment 34. The peptide of any one of embodiments 1-33, wherein said second anti-CD3 dimerizing domain is bound to an Fc domain through a third chemical linker.

Embodiment 35. The peptide of any one of embodiments 1-34, wherein said CD38 binding domain is capable of binding a CD3 protein with an equilibrium dissociation constant (KD) of less than 550 nM.

Embodiment 36. The peptide of any one of embodiments 1-35, wherein said first chemical linker and said second chemical linker are independently a covalent linker or a non-covalent linker.

Embodiment 37. The peptide of any one of embodiments 1-36, wherein said first chemical linker and said second chemical linker are independently a cleavable peptide linker.

Embodiment 38. The peptide of any one of embodiments 1-37, wherein said first chemical linker and said second chemical linker are independently an enzymatically cleavable linker.

Embodiment 39. The peptide of any one of embodiments 1-38, wherein said first chemical linker and said second chemical linker are independently a protease cleavable linker.

Embodiment 40. The peptide of any one of embodiments 1-39, wherein said first chemical linker and said second chemical linker independently have a length of about 0 to about 15 amino acid residues.

Embodiment 41. The peptide of any one of embodiments 1-40, wherein said first chemical linker comprises the sequence of SEQ ID NO:9.

Embodiment 42. The peptide of any one of embodiments 1-41, wherein said second chemical linker comprises the sequence of SEQ ID NO:10, SEQ ID NO:11, SEQ ID NO:30 or SEQ ID NO:31.

Embodiment 43. The peptide of any one of embodiments 1-42, wherein said CD38 ligand binding domain is a single domain antibody.

Embodiment 44. The peptide of any one of embodiments 1-43, wherein said CD38 ligand binding domain comprises a CDR L1 as set forth in SEQ ID NO:12, a CDR L2 as set forth in SEQ ID NO:13 and a CDR L3 as set forth in SEQ ID NO: 14; a CDR L1 as set forth in SEQ ID NO:22, a CDR L2 as set forth in SEQ ID NO:23 and a CDR L3 as set forth in SEQ ID NO: 24; or a CDR L1 as set forth in SEQ ID NO:26, a CDR L2 as set forth in SEQ ID NO:27 and a CDR L3 as set forth in SEQ ID NO:28.

Embodiment 45. The peptide of any one of embodiments 1-44, wherein said CD38 ligand binding domain comprises the sequence of SEQ ID NO:15, SEQ ID NO:25 or SEQ ID NO: 29.

Embodiment 46. The peptide of any one of embodiments 1-45, wherein said peptide comprises the sequence of SEQ ID NO:16, SEQ ID NO:17, SEQ ID NO:18, SEQ ID NO:19, SEQ ID NO:20 or SEQ ID NO:21.

Embodiment 47. The peptide of any one of embodiments 1-46, wherein said CD38 ligand binding domain is capable of binding a CD38 protein with an equilibrium dissociation constant (KD) of less than 1.5 nM.

Embodiment 48. The peptide of embodiment 1-47, wherein said peptide forms part of a T cell.

Embodiment 49. An isolated nucleic acid encoding a peptide of any one of embodiments 1-47.

Embodiment 50. An expression vector comprising the nucleic acid of embodiment 49.

Embodiment 51. The expression vector of embodiment 50, wherein said expression vector is a viral vector.

Embodiment 52. A T lymphocyte comprising the expression vector of any one of embodiments 1-47.

Embodiment 53. A method of treating cancer in a subject in need thereof, said method comprising administering to a subject a therapeutically effective amount of a peptide of any one of embodiments 1-47, thereby treating cancer in said subject.

Embodiment 54. The method of embodiment 53, wherein said cancer is acute myelogenous leukemia (AML), multiple myeloma (MM), lymphoma, T-cell acute lymphoblastic leukemia (T-ALL), or prostate cancer.

Embodiment 55. A pharmaceutical composition comprising a therapeutically effective amount of a peptide of any one of embodiments 1-47, and a pharmaceutically acceptable excipient.

1. A peptide comprising:
(i) a first anti-CD3 dimerizing domain bound to a CD38 binding domain through a first chemical linker; and
(ii) a second anti-CD3 dimerizing domain bound to said CD38 binding domain through a second chemical linker;
wherein said first anti-CD3 dimerizing domain is capable of non-covalently binding to said second anti-CD3 domain to form an anti-CD3 binding domain.

2. (canceled)

3. The peptide of claim 1, wherein said first anti-CD3 dimerizing domain is bound to said second anti-CD3 dimerizing domain.

4. (canceled)

5. (canceled)

6. The peptide of claim 5, wherein said first anti-CD3 dimerizing domain comprises a variable light chain domain.

7. The peptide of claim 6, wherein said first anti-CD3 dimerizing domain comprises a constant light chain domain.

8. (canceled)

9. (canceled)

10. (canceled)

11. The peptide of claim 7, wherein said second anti-CD3 dimerizing domain comprises a constant heavy chain domain.

12. (canceled)

13. The peptide of claim 11, wherein said second anti-CD3 dimerizing domain is an antibody heavy chain.

14. The peptide of claim 5, wherein said first anti-CD3 dimerizing domain comprises a variable heavy chain domain.

15. The peptide of claim 14, wherein said first anti-CD3 dimerizing domain comprises a constant heavy chain domain.

16. (canceled)

17. (canceled)

18. The peptide of claim 17, wherein said second anti-CD3 dimerizing domain comprises a variable light chain domain.

19. The peptide of claim 18, wherein said second anti-CD3 dimerizing domain comprises a constant light chain domain.

20. (canceled)

21. (canceled)

22. The peptide of claim 1, wherein said anti-CD3 binding domain is a Fab domain.

23. The peptide of claim 1, wherein said first anti-CD3 dimerizing domain is an antibody light chain comprising a CDR L1 as set forth in SEQ ID NO: 1, a CDR L2 as set forth in SEQ ID NO:2 and a CDR L3 as set forth in SEQ ID NO:3.

24. (canceled)

25. The peptide of claim 1, wherein said first anti-CD3 dimerizing domain is an antibody heavy chain comprising a CDR H1 as set forth in SEQ ID NO: 4, a CDR H2 as set forth in SEQ ID NO:5, and a CDR H3 as set forth in SEQ ID NO:6.

26. (canceled)

27. The peptide of claim 1, wherein said second anti-CD3 dimerizing domain is an antibody heavy chain comprising a CDR H1 as set forth in SEQ ID NO: 4, a CDR H2 as set forth in SEQ ID NO:5, and a CDR H3 as set forth in SEQ ID NO:6.

28. (canceled)

29. The peptide of claim 1, wherein said second anti-CD3 dimerizing domain is an antibody light chain comprising a CDR L1 as set forth in SEQ ID NO: 1, a CDR L2 as set forth in SEQ ID NO:2 and a CDR L3 as set forth in SEQ ID NO:3.

30. The peptide of claim 1, wherein said first anti-CD3 dimerizing domain is an antibody light chain comprising a CDR L1 as set forth in SEQ ID NO: 32, a CDR L2 as set forth in SEQ ID NO:33 and a CDR L3 as set forth in SEQ ID NO: 34.

31. The peptide of claim 1, wherein said second anti-CD3 dimerizing domain is an antibody heavy chain comprising a CDR H1 as set forth in SEQ ID NO: 35, a CDR H2 as set forth in SEQ ID NO:36 and a CDR H3 as set forth in SEQ ID NO: 37.

32. (canceled)

33. (canceled)

34. (canceled)

35. (canceled)

36. (canceled)

37. The peptide of claim 1, wherein said first chemical linker and said second chemical linker are independently a cleavable peptide linker.

38. (canceled)

39. (canceled)

40. (canceled)

41. (canceled)

42. (canceled)

43. The peptide of claim 1, wherein said CD38 ligand binding domain is a single domain antibody.

44. The peptide o of claim 1, wherein said CD38 ligand binding domain comprises a CDR L1 as set forth in SEQ ID NO:12, a CDR L2 as set forth in SEQ ID NO: 13 and a CDR L3 as set forth in SEQ ID NO:14; a CDR L1 as set forth in SEQ ID NO: 22, a CDR L2 as set forth in SEQ ID NO:23 and a CDR L3 as set forth in SEQ ID NO: 24; or a CDR L1 as set forth in SEQ ID NO:26, a CDR L2 as set forth in SEQ ID NO:27 and a CDR L3 as set forth in SEQ ID NO:28.

45. (canceled)

46. The peptide of claim 1, wherein said peptide comprises the sequence of SEQ ID NO: 16, SEQ ID NO: 17, SEQ ID NO: 18, SEQ ID NO: 19, SEQ ID NO: 20 or SEQ ID NO:21.

47. (canceled)

48. The peptide of claim 1, wherein said peptide forms part of a T cell.

49. An isolated nucleic acid encoding a peptide of claim 1.

50. An expression vector comprising the nucleic acid of claim 49.

51. (canceled)

52. A T lymphocyte comprising the expression vector of claim 50.

53. A method of treating cancer in a subject in need thereof, said method comprising administering to a subject a therapeutically effective amount of a peptide of claim 1, thereby treating cancer in said subject.

54. (canceled)

55. A pharmaceutical composition comprising a therapeutically effective amount of a peptide of claim 1, and a pharmaceutically acceptable excipient.
